(19) United States
(12) Patent Application Publication
(10) Pub. No.: US 2023/0235315 A1
(43) Pub. Date: Jul. 27, 2023

(54) METHOD FOR PRODUCING GENETICALLY MODIFIED CELLS

(71) Applicant: Horizon Discovery Limited, Cambridge (GB)

(72) Inventors: John Lambourne, Cambridge (GB); Immacolata Porreca, Cambridge (GB)

(21) Appl. No.: 18/011,311

(22) PCT Filed: Jul. 9, 2021
(86) PCT No.: PCT/GB21/51775

§ 371 Date: Dec. 19, 2022

(30) Foreign Application Priority Data
Jul. 10, 2020 (GB) 2010689.4
Sep. 25, 2020 (GB) 2015204.7

(51) Int. Cl. C12N 15/10; C12N 5/0783; C12N 9/24

(52) U.S. Cl. CPC C12N 15/102; C12N 5/0636; C12Y 302/02027; C12N 9/2497; C12Y 305/04004; C07K 2319/00; C12N 2510/00

(57) ABSTRACT

A method for producing genetically engineered immune cells, e.g. T cells, or iPSCs which uses an RNA-scaffold mediated base editing system. The method enables precise modifications to be made to the genome whilst minimizing the possibility of off-target effects, making the method particularly suitable for therapeutic applications.

### CROSS-REFERENCE TO RELATED APPLICATIONS

This application is a national stage application of PCT/GB2021/051775, filed Jul. 9, 2021, which claims the benefit of both the filing date of GB 2010689.4, filed Jul. 10, 2020 and the filing date of GB 2015204.7, filed Sep. 25, 2020, the entire disclosures of which are incorporated by reference as if set forth fully herein.

### FIELD OF THE INVENTION

The invention relates to a method for producing genetically modified cells, e.g. primary cells, particularly immune cells e.g. T-cells, using RNA-mediated base editing.

### BACKGROUND

The precise modulation of primary human cells has multiple applications in the fields of immunotherapy, autoimmunity, enzymopathy, and disease phenotype correction. Modulation of patient immune cells at the genetic level is an attractive route for therapy due to the permanency of treatment and the low risk of rejection by the patient. One approach for the gene editing of immune cells is to use Clustered Regularly Interspaced Short Palindromic Repeat (CRISPR) systems to induce a double stranded break (DSB) within a gene of interest, thereby resulting in the formation of small insertions or deletions (collectively referred to as ‘indels’) created by highly variable repair via the Non-Homologous End Joining (NHEJ) pathway. Alternatively, precise genome alterations can be achieved by introduction of a DSB along with co-delivery of a DNA template for repair via homology directed repair (HDR). While this approach is efficient and reliable when simply disrupting a single gene by NHEJ, precision alteration of single nucleotides by HDR is far less efficient. Furthermore, inducing multiple DSBs during multiplexed gene editing procedures can cause undesirable genotoxicity and the formation of potentially oncogenic gross chromosomal translocations. Accordingly, there remains a need in the field for more controlled and safer methods of multiplexed genetic engineering of human immune cells with limited induction of toxic DSBs.

WO2019/178225 (Moriarity) describes methods and systems for lymphohematopoietic cell engineering using cas9 base editors. It discloses a system comprising a cas nickase directly fused to a deaminase enzyme and guide RNAs designed to target splice acceptor-splice donor sites. This base editing system is known as the BE system.

A newly developed, RNA-aptamer mediated base editing system, is described in WO2017/011721 (Rutgers) entitled: ‘Nuclease-independent targeted gene editing platform and uses thereof’. In this system, the effector deaminase is recruited via an RNA interaction and is not fused directly to cas nickase. This system, is an RNA scaffold mediated base editing system that, contains a modified gRNA with a re-programmable RNA-aptamers at the 3′ end, which recruits the cognate aptamer ligand fused to an effector (such as a deaminase effector). Using this system, targeted nucleotide modification was achieved with high precision in prokaryotic cells and eukaryotic cells including mammalian cells; see also WO2018129129. A second generation RNA scaffold mediated base editing system with increased specificity and efficacy in prokaryotic cells was tested and further improved in mammalian cells and is disclosed in WO2021055459 (U.S. application 62/901,584).

The present inventors have developed a new method for producing genetically engineered immune cells, e.g. T cells, which uses an RNA-scaffold mediated base editing system. The method and system provided herein enable precise modifications to be made to the genome whilst minimizing the possibility of off-target effects, making the method and system particularly suitable for therapeutic applications.

### SUMMARY OF THE INVENTION

In a first aspect the invention provides a method for genetically modifying an immune cell, the method comprising introduction into the cells and/or expression in the cells of:

i) a sequence-targeting component comprising a sequence-targeting protein;

ii) an RNA scaffold comprising
(a) a crRNA comprising a guide RNA sequence that is complementary to a target nucleic acid sequence,
(b) a tracrRNA capable of binding to the sequence-targeting protein, and
(c) an RNA motif; and

(iii) an effector fusion protein comprising
a) an RNA binding domain capable of binding to the RNA motif,
b) a linker, and
c) an effector domain having a cytosine deamination activity or adenine deamination activity; and
culturing the introduced cell to produce a genetically modified immune cell.

The method may use a sequence-targeting component comprising a sequence-targeting protein fused to one or more uracil DNA glycosylase (UNG) inhibitor peptide(s) (UGI).

In any embodiment the RNA scaffold may comprises one or more RNA, or one or two RNA motifs. In any embodiment the sequence-targeting component and/or the effector fusion protein may comprise one or more nuclear localization signals (NLSs).

The sequence-targeting protein is a Type II Cas protein. For example, the sequence-targeting protein is a Type II Cas protein that is nuclease null or has nickase activity and/or may comprise the sequence of dCas9 or nCas9, for example of a species selected from the group consisting of Streptococcus pyogenes, Streptococcus agalactiae, Staphylococcus aureus, Streptococcus thermophilus, Streptococcus thermophilus, Neisseria meningitidis, and Treponema denticola.

The methods provided herein may use an RNA motif and the RNA binding domain pair selected from the group consisting of: a telomerase Ku binding motif and Ku protein or an RNA-binding section thereof, a telomerase Sm7 binding motif and Sm7 protein or an RNA-binding section thereof, a MS2 phage operator stem-loop and MS2 coat protein (MCP) or an RNA-binding section thereof, a PP7 phage operator stem-loop and PP7 coat protein (PCP) or an RNA-binding section thereof, a SfMu phage Com stem-loop and Com RNA binding protein or an RNA-binding section thereof.

The methods provided herein may use an effector domain having cytosine deamination activity or cytidine deamination activity (the terms are used interchangeably), for example, a wild type or genetically engineered version of AID, CDA, APOBEC1, APOBEC3A, APOBEC3B, APOBEC3C, APOBEC3D, APOBEC3F, or other APOBEC family enzymes.

The methods provided herein may use an effector domain having adenine deamination activity or adenosine deamination activity (the terms are used interchangeably), for example, a wild type or genetically engineered version of ADA, ADAR family enzymes, or tRNA adenosine deaminases.

The methods provided herein may be used to modify a target nucleic acid that is extrachromosomal DNA or genomic DNA on a chromosome.

The methods provided herein may be used to modify an immune cell selected from a T cell (including a primary T cell), Natural Killer (NK cell), B cell, or CD34+ hematopoietic stem and progenitor cells (HSPCs), such as HSPCs isolated from umbilical cord blood or bone marrow and cells differentiated from them. In a particular embodiment the immune cell is differentiated from an induced pluripotent stem cell (iPSC). The immune cell may be an engineered immune cell, such as T-cell comprising a CAR or TCR.

The methods herein may thus be applied to further engineer a cell that has already been modified to include a CAR and/or TCR that is useful in therapy. Alternatively, as will be apparent to the skilled person, the present methods may be used to introduce single or multiple genetic modifications into an immune cell or iPSC which is then followed by the introduction of further mutations using conventional gene editing methods. For example, CRISPR mediated homology directed repair (HDR) may be used to introduce (knock-in) one or more exogeneous nucleotide sequences into the genome of cells that have already been genetically modified using the methods provided herein.

The methods provided herein involve a genetic modification that corrects a genetic mutation or inactivates the expression of a gene or changes the expression levels of a gene or changes intron-exon splicing. The genetic modification may be a point mutation, optionally wherein the point mutation introduces a premature stop codon, disrupts a start codon, disrupts a splice site or corrects a genetic mutation.

The guide RNA sequence used in the methods provided herein may include a SA-SD sequence, for example, a SA-SD guide RNA sequence selected from the sequences set forth in Table 4.

The RNA scaffold used in the methods herein may be introduced into the cell as chemically synthesised RNA molecule. The RNA scaffold may comprise one or more modifications as described herein below.

For example, guide RNA sequence may be chemically modified to include a 2′-(9-methyl) phosphorothioate modification on at least one 5′ nucleotide and/or at least one 3′ nucleotide of the guide RNA sequence.

The RNA scaffold may be synthesised as a single molecule, or synthesised or expressed as two separate components, optionally, wherein the first component comprises a) the crRNA and the second component comprises b) the tracrRNA and c) the RNA motif. The two components may then be allowed to hybridise prior to introduction into the cell.

The methods provided herein may utilise one or more RNA motifs located at the 3′ end of the RNA scaffold.

The RNA motif may be an MS2 aptamer, for example, an MS2 aptamer that has an extended stem, for example, an extended stem comprising 2-24 nucleotides.

The methods provided herein may utilise a targeting fusion protein that includes nCas9 or dCas9 with one or two UGIs and an RNA scaffold with a single RNA motif, for example, a single MS2 located at the 3′ end of the RNA scaffold.

The methods provided herein may target different genes in the immune cells to, for example, introduce a genetic modification that results in the desired base change and/or subsequent phenotypic loss of any of the following proteins TRAC, TRBC1, TRBC2, PDCD1, CD52 and B2M. The methods may be used to edit one or both alleles of a target gene in an immune cell. The methods provided herein may be used to edit multiple different genes (multiplex base editing), and successfully edit one or both alleles of the target genes. For example, the method may use multiple RNA scaffolds comprising different guide RNA sequences to genetically modify (base edit) multiple different genetic loci, (e.g. 2 to 10) or at least 3 or at least 4 loci. The multiple RNA scaffolds may be one component and/or two component RNA scaffolds.

Advantageously, it has been shown herein that the system can be used to base edit multiple genes to produce functional knock-outs, for example by the introduction of point mutations to one or both alleles of the target genes.

The methods provided herein may use the components in the form of RNA or protein, for example the sequence-targeting component, the RNA scaffold and the effector fusion protein are all introduced into the cell as RNA or protein; this means that the method/system provided herein could, if desired, be used as a vector-free system. The sequence-targeting component, RNA scaffold and the effector fusion protein may be all introduced into the cell as RNA using electroporation.

Also provided herein are genetically modified immune cells obtained according to the methods described herein. Also disclosed herein is a system for genetically modifying an immune cell comprising i) a sequence-targeting component, ii) a RNA scaffold, and (iii) an effector fusion protein, as used in any of the genetic modification methods described herein.

### FIGURES

FIG. 1: Illustrated in FIGS. 1A and 1B are schematics of an exemplary base editing system for use in the methods provided herein. 1 exemplifies a sequence targeting component (such as dCas9 or nCas9_{D10A}), 2 exemplifies a chimeric RNA scaffold containing a guide RNA motif (2.1) (crRNA for sequence targeting), a CRISPR motif (2.2) (tracrRNA for Cas protein binding), and a recruiting RNA aptamer motif (2.3) (RNA motif for recruiting effector-RNA binding protein fusion); 3 exemplifies a fusion protein comprising an effector (3.1) (e.g., cytidine deaminase) fused to an RNA aptamer ligand (3.2).

FIG. 2: Assessment of Phenotype Variation as consequence of gene disruption at TRAC locus due to viral-delivered sgRNA-Aptamers by Cytosine to Thymine Base Changing.

TRAC protein level as determined by flow cytometry for TCR a/b following disruption of TRAC exon 3 splice acceptor site. Data are reported as percentage normalised on the protein level in control cells. The gRNA fused to 2×MS2 aptamers and targeting the splice acceptor site of exon 3 was delivered into CD3 positive T-cells by lentiviral transduction. AID Deaminases-based editor mRNA was delivered via electroporation in transduced T-cells and protein level was determined 72-96 hours post-delivery.

FIG. 3: TRAC Exon1 Splice Donor Site Base Change Variation Due to viral-delivered sgRNA-Aptamers by Cytosine to Thymine Base Changing.

Quantification of C to T conversion of target base within the exon 1 splice donor site. The gRNA fused to 2×MS2 aptamers and targeting the splice donor site of exon 1 was delivered into CD3 positive T-cells by lentiviral transduction. AID Deaminases-based editor mRNA was delivered via electroporation in transduced T-cells and base conversion was determined 72-96 hours post mRNA delivery by analysis of Sanger sequencing traces. The entire protospacer is reported in the drawing with the splice acceptor site highlighted in the box.

FIG. 4: Assessment of Phenotype Variation as consequence of gene disruption at TRAC locus due to viral-delivered sgRNA-Aptamers by Cytosine to Thymine Base Changing.

TRAC protein level as determined by flow cytometry for TCR a/b following disruption of TRAC exon 1 splice donor site. Data are reported as percentage normalised on the protein level in control cells. The gRNA fused to 2×MS2 aptamers and targeting the splice donor site of exon 1 was delivered into CD3 positive T-cells by lentiviral transduction. AID Deaminases-based editor mRNA was delivered via electroporation in transduced T-cells and protein level was determined 72-96 hours post-delivery.

FIG. 5: TRAC Exon3 Splice Acceptor Site Base Change Variation Due to Synthetic two-part crRNA:tracrRNA-Aptamers by Cytosine to Thymine Base Changing.

Quantification of C to T conversion of target base within the exon 3 splice acceptor site. Synthetic crRNA:tracrRNA (with and without aptamers) and Apobec1 or AID Deaminases-based editor mRNAs were co-delivered, via electroporation, into CD3 positive T-cells and base conversion was determined 72-96 hours post-delivery by analysis of Sanger sequencing traces. The entire protospacer is reported in the drawing with the splice acceptor site highlighted in the box.

FIG. 6: Assessment of Phenotype Variation as consequence of gene disruption at TRAC locus due Synthetic two-part crRNA:tracrRNA-Aptamers by Cytosine to Thymine Base Changing.

TRAC protein level as determined by flow cytometry for TCR a/b following disruption of TRAC exon 3 splice acceptor site. Data are reported as percentage normalised on the protein level in control cells.

Synthetic crRNA:tracrRNA (with and without aptamers) and Apobec1 or AID Deaminases-based editor mRNAs were co-delivered, via electroporation, into T-cells and protein level was determined 72-96 hours post-delivery.

FIG. 7: Triplex Gene KO Base Change with Synthetic two-part crRNA:tracrRNA-Aptamers by Cytosine to Thymine Base Changing in Primary Immune Cells.

Quantification of C to T conversion of target base within the target guide sequence for the gene KO of: TRAC, B2M, and CD52. Synthetic crRNA:tracrRNA-aptamer complexes and Apobec1 Deaminase-based editor mRNAs were co-delivered, via electroporation, into CD3 positive T-cells and base conversion was determined 72-96 hours post-delivery by analysis of Sanger sequencing traces. The entire protospacer is reported in the drawing with the target site highlighted in the boxes.

FIG. 8: Assessment of Phenotype Change as Consequence of Multigene Triple Gene Disruption by Synthetic two-part crRNA:tracrRNA-Aptamers due Cytosine to Thymine Base Changing.

Multistain flow cytometry panel displaying the levels of single, double, and triple functional gene KO in a Pan T cell population. Synthetic crRNA:tracrRNA-aptamer complexes and Apobec1 Deaminase-based editor mRNAs were co-delivered, via electroporation, into CD3 positive T-cells and multistain flow cytometry was used 72-96 hours post-delivery to determine functional KO.

The figure displays the data for all cells that displayed KO of at least one gene, unedited population accounted for 19.4% of the population in this example, so that 80.6% is displayed in the figure which includes single-only (White), double-only (Grey), and triple KO (Black). The data shows that ˜10% of the edited population is Triple functional KO at this time point, which displays the functionality of this technology to be used as a multi-loci editing tool.

FIG. 9: Quadruplex Gene KO Base Change with Synthetic two-part crRNA:tracrRNA-Aptamers by Cytosine to Thymine Base Changing in Primary Immune Cells.

Quantification of C to T conversion of target base within the target guide sequence for the gene KO of: TRAC, B2M, CD52 and PD1. Synthetic crRNA:tracrRNA-aptamer complexes and Apobec1 Deaminase-based editor mRNAs were co-delivered, via electroporation, into CD3 positive T-cells and base conversion was determined 72-96 hours post-delivery by analysis of Sanger sequencing traces. The entire protospacer is reported in the drawing with the target site highlighted in the boxes.

FIG. 10: Quadruplex Gene KO Base Change with Synthetic one-part sgRNA-Aptamers by Cytosine to Thymine Base Changing in Primary Immune Cells.

Quantification of C to T conversion of target base within the target guide sequence for the gene KO of: TRAC, B2M, CD52, and PD1. Synthetic sgRNA-aptamer complexes and Apobec1 Deaminase-based editor mRNAs were co-delivered, via electroporation, into CD3 positive T-cells and base conversion was determined 72-96 hours post-delivery by analysis of Sanger sequencing traces. The entire protospacer is reported in the drawing with the target site highlighted in the boxes.

FIG. 11: Assessment of Phenotype Change as Consequence of Multigene Quadruple Gene Disruption by Synthetic two-part crRNA:tracrRNA-Aptamers and one-part sgRNA-Aptamers due to Cytosine to Thymine Base Changing at Two Post-Electroporation Timepoints Multistain flow cytometry panel displaying the levels quadrplex gene KO in a Pan T cell population at Day 3 and Day 7 post-electroporation. Synthetic crRNA:tracrRNA-aptamer or sgRNA-aptamer complexes and Apobec1 Deaminase-based editor mRNAs were co-delivered, via electroporation, into CD3 positive T-cells and multistain flow cytometry was used a Day 3 (72-96 hours) and at Day 7 (168-193 hours) post-delivery to determine functional KO.

The figure displays the data for all cells that displayed KO of at least one gene in the population; live unedited cells were removed from the comparison (Unedited population percentage: Day 3 crRNA:tracrRNA-aptamer=43.5%, Day 3 sgRNA-aptamer=32.3%, Day 7 crRNA:tracrRNA-aptamer=21.7%, Day 7 sgRNA-aptamer=16.1%). From the edited live population the percentage of quadruplex KO is displayed as a percentage of all the edited populations (e.g. Single-only (TRAC, B2M, CD52, +PD-1), double-only (TRAC-B2M, TRAC-CD52, TRAC-PD-1, B2M-CD52, B2M-PD-1, +CD52-PD-1), triple-only (TRAC-B2M-CD52, TRAC-B2M-PD-1, +B2M-CD52-PD-1), and Quadruplex (All TRAC—The data shows for sgRNA-aptamers that that ˜10% of the edited population is Quadruplex functional KO at Day 3 and increases to ˜35% at Day 7. The data shows for crRNA:tracrRNA-aptamers that that ˜3% of the edited population is Quadruplex functional KO at Day 3 and increases to −7% at Day 7.

FIG. 12: Quadruplex Gene KO Base Change with Synthetic one-part sgRNA-Aptamers by Cytosine to Thymine Base Changing in Primary Immune Cells from multiple donors. Quantification of C to T conversion of target base within the target guide sequence for the gene KO of: TRAC (A), B2M (B), CD52 (C) and PDCD1 (D). Synthetic sgRNA-aptamer complexes and Apobec1 Deaminase-based editor mRNAs were co-delivered, via electroporation, into CD3 positive T-cells and base conversion was determined 5-7 days post-delivery by analysis of Sanger sequencing traces. The entire protospacer is reported in the drawing with the target site highlighted in the boxes. Data are reported for three donors.

FIG. 13: Assessment of Phenotype Change as Consequence of Multigene Gene (4) Disruption by Synthetic one-part sgRNA-Aptamers due to Cytosine to Thymine Base Changing in multiple donors. Multistain flow cytometry panel displaying the fraction of cells KO in one, two, three, four genes or unedited in Pan T cell populations at Day 7 post-electroporation. Synthetic sgRNA-aptamer complexes and Apobec1 Deaminase-based editor mRNAs were co-delivered, via electroporation, into CD3 positive T-cells and multistain flow cytometry was used at Day 7 post-delivery to determine functional KO. The figure displays the data for all cells: Single KO (TRAC KO+B2M KO+CD52 KO+PD-1 KO), double KO (TRAC-B2M KO+TRAC-CD52 KO+TRAC-PD-1 KO+B2M-CD52 KO+B2M-PD-1 KO+CD52-PD-1 KO), triple KO (TRAC-B2M-CD52 KO+TRAC-B2M-PD-1 KO+B2M-CD52-PD-1 KO+TRAC-CD52-PD1 KO), and Quadruplex KO (All TRAC-B2M-CD52-PD-1). Data are reported as mean and SD of three donors. The data shows that ˜40% of the live cell population is Quadruplex functional KO, ˜35% is triple KO and ˜15% is double KO at Day 7.

FIG. 14: Comparison of Outcomes and Product Purity of Cytosine Deaminase Base Editing Technology by NGS.

Next generation sequencing (NGS) of the four targets for quadruplex editing target sites: B2M, CD52, TRAC, and PDCD1. Target base highlighted and referred to the cytosine within the editing window that is targeted for C-to-T conversion to enable splice site disruption and subsequent gene knockout. Control refers to cells exposed to electroporation only (no gene editing reagents), and BE refers to cells electroporated with nCas9-UGI-UGI/Apobec1 base editor. Data are shown as the mean of n=3 biological replicates from three independent experiments performed by three different operators.

FIG. 15: Comparison of Indel formation of Base Editing Technology at Guide-Specific On-targets in Comparison to Equivalent Nuclease Active Cas9 Technology by NGS.

Next generation sequencing (NGS) of the four targets for quadruplex editing target sites: B2M, CD52, TRAC, and PDCD1. INS and DEL refer to percentage insertion and percentage deletion across the 20 bp target region, respectively; note that insertion and deletion rates are not mutually exclusive. Control refers to cells exposed to electroporation only (no gene editing reagents), BE refers to cells electroporated with nCas9-UGI-UGI/Apobec1 base editor, and Cas9 refers to cells treated with the equivalent nuclease Cas9 technology. For both BE and Cas9 conditions, cells were also exposed to four sgRNA targeting B2M, CD52, TRAC and PDCD1. Data are shown as the mean of n=3 biological replicates.

FIG. 16: Comparison of Base Editing Technology at Guide-Specific Off-targets in Comparison to Equivalent Nuclease Active Cas9 Technology by NGS.

Next generation sequencing (NGS) of the two found guide-specific off-target sites. A) INS and DEL refer to percentage insertion and percentage deletion across two predicted gDNA off target sites (20-21 bp region), respectively. B) Target base refers to the cytosine within the editing window that shows the highest level of base conversion within the predicted off target region. A) and B) Control refers to cells exposed to electroporation only (no gene editing reagents), BE refers to cells electroporated with nCas9-UGI-UGI/Apobec1 base editor, and Cas9 refers to cells treated with the equivalent nuclease Cas9 technology. For both BE and Cas9 conditions, cells were also exposed to four sgRNA targeting B2M, CD52, TRAC and PDCD1. OT1 refers to a B2M sgRNA-associated predicted gDNA off target site and OT2 refers to a PDCD1 sgRNA-associated predicted gDNA off target site. Data are shown as the mean of n=3 biological replicates from three independent experiments.

FIG. 17: Triplex Gene KO Base Change with Synthetic one-part sgRNA-Aptamers by Cytosine to Thymine Base Changing in Primary Immune Cells from multiple donors. A/B/C) Quantification of C to T conversion of target base within the target guide sequence for the gene KO of: B2M, CD52 and PD1. The entire protospacer is reported in the drawing with the target site highlighted in the boxes. D) Flow cytometry analysis of triple KO percentage of the total live cells. Synthetic sgRNA-aptamer complexes and Apobec1 Deaminase-based editor mRNAs were co-delivered, via electroporation, into CD3 positive T-cells and base conversion was determined 5-7 days post-delivery by analysis of Sanger sequencing traces.

FIG. 18: Functional Assessment of Multiplex Edited T cells by Tumor Cell Killing and Proliferation. A) Bispecific antibody induced tumor cell lysis, as measured by flow cytometry to assess viability of Daudi cells. B) Bispecific antibody and tumor cell induced T cell proliferation as assessed by CellTracer CSFE staining and flow cytometry

FIG. 19: Functional Assessment of Multiplex Edited T cells by Cytokine Release Assays. Q-beads were used to determine the absolute amounts of cytokine found in the final culture supernatant at the end point of the assay. A) Assessment of Tumor Necrosis Factor Alpha (TNFa) B) Assessment of Interferon Gamma (IFNg)

FIG. 20: Assessment of Anti-CD19 CAR Surface Phenotype of Transduced T cell in Multiplex Base Edited Samples and Unedited Controls.

Multistain flow cytometry panel displaying the percentage of T cells positive for the Anti-CD19 CAR. Cells have been transduced with a lentivirus carrying an Anti-CD19 CAR and then electroporated with the base editor components. As a control, cells transduced with the same lentivirus but not electroporated with the base editing component are reported. Further controls are cells transduced with an Empty lentivirus (which only contains Puromycin gene) and electroporated or not with the base editing components.

FIG. 21: Assessment of Phenotype Change as Consequence of Multigene Quadruplex Gene Disruption, in transduced T cells, by Synthetic one-part sgRNA-Aptamers due to Cytosine to Thymine Base Changing.

Multistain flow cytometry panel displaying the Anti-CD19 CAR positive cells that are also single, double, triple, and quadruple functional gene KO in a Pan T cell population. Synthetic sgRNA-aptamer complexes and Apobec1 Deaminase-based editor mRNAs were co-delivered, via electroporation, into CD3 positive Anti-CD19 CAR transduced T-cells and multistain flow cytometry was used 72-96 hours post-delivery to determine functional KO. A) Displays AntiCD19 CAR transduced where samples had base editing technology reagents applied. B) Displays AntiCD19 CAR transduced where samples had base editing technology reagents not applied.

FIG. 22: Quadruplex Gene KO Base Change with Synthetic one-part sgRNA-Aptamers by Cytosine to Thymine Base Changing in Anti-CD19 CAR Transduced Primary Immune Cells.

Quantification of C to T conversion of target base within the target guide sequence for the gene KO of: TRAC, B2M, CD52, and PDCD1. Synthetic sgRNA-aptamer complexes and Apobec1 Deaminase-based editor mRNAs were co-delivered, via electroporation, into CD3 positive T-cells and base conversion was determined 72-96 hours post-delivery by analysis of Sanger sequencing traces. The entire protospacer is reported in the drawing with the target site highlighted in the boxes.

FIG. 23: Assessment of Phenotype Variation as consequence of gene disruption at B2M locus by Synthetic one-part sgRNA-Aptamers due to Cytosine to Thymine Base Changing Functional knockout of B2M as quantified by flow cytometry. Synthetic sgRNA-aptamer complexes and Apobec1 Deaminase-based editor mRNAs were co-delivered, via electroporation, into iPSCs and B2M knockout was determined 72-96 hours post-delivery. Edited population included the sgRNA and the Control population included the same reagents but no sgRNA.

FIG. 24: Multiplex Gene Editing with Synthetic one-part sgRNA-Aptamers by Cytosine to Thymine Base Changing in human iPSCs.

Quantification of C to T conversion of target base within the target guide sequence for the gene KO of: TRAC, B2M, CD52, and PDCD1. Synthetic sgRNA-aptamer complexes and Apobec1 Deaminase-based editor mRNAs were co-delivered, via electroporation, into iPSCs and base conversion was determined 96-120 hours post-delivery by analysis of Sanger sequencing traces. The entire protospacer is reported in the drawing with the target site highlighted in the boxes.

Multiplex Gene Editing with Synthetic sgRNA-Aptamers by Cytosine to Thymine Base Changing in human iPSCs.

### DETAILED DESCRIPTION OF THE INVENTION

This invention relates to a new method for targeted genome modification of immune cells. This invention is based, at least in part, on use of an RNA-aptamer mediated base editing system. The system comprises the following functional components: (1) CRISPR/Cas-based module (that has no double strand cleavage activity but may have nickase activity) engineered for sequence targeting; (2) an RNA scaffold-based module for guiding the platform to a target sequence as well as for recruitment of a correction module; and (3) a non-nuclease DNA/RNA modifying enzyme as an effector correction module, such as cytidine deaminases (e.g., activation-induced cytidine deaminase, AID).

In an aspect, provided herein is a method for producing a genetically modified immune cell. In particular, the method comprises introducing base editing components into an immune cell, where the components comprise: (i) a sequence-targeting protein, or a polynucleotide encoding the same, (ii) an RNA scaffold, or a DNA polynucleotide encoding the same, and (iii) a non-nuclease effector fusion protein, or a polynucleotide encoding the same. The RNA scaffold guides the sequence-targeting protein and the effector fusion protein to a target polynucleotide at a target site and the effector domain of the effector fusion protein modifies the sequence. As disclosed herein, the sequence-targeting protein, such as a Cas9 protein, is modified such that its double-strand cleavage activity is eliminated.

RNA-Mediated Base Editing System

Conventional nuclease-dependent precise genome editing for correction of mutations usually requires introduction of DNA double strand breaks (DSBs) and activation of the homology dependent repair (HDR) pathway.

Recently an RNA-mediated base editing system was also developed. This system recruits a base editing enzyme to a target DNA sequence through the RNA component of a CRISPR complex. This system contains a modified gRNA with a re-programmable RNA-aptamer at the 3′ end, which recruits the cognate aptamer ligand fused to an effector (such as a deaminase effector). Using this system, targeted nucleotide modification was achieved with high precision in prokaryotic cells and eukaryotic cells including mammalian cells; see WO2018129129 and WO2017011721. As also disclosed herein, a new, second generation base editing system with increased specificity and efficacy in prokaryotic cells was tested and further improved in mammalian cells. The second generation system/platform exhibits high specificity, high efficiency, and low off-target liability. With a modular design that fully separates the nucleic acid modification module from the nucleic acid recognition module as well as other advantages disclosed herein, the RNA-mediated base editing platform provides an alternative to recruitment of the effector through fusion to or direct interaction with the sequence-targeting protein, which could not effectively separate sequence-targeting function from nucleic acid modification function. Devoid of the requirements of DNA DSB and HDR, the new system provides powerful tools for genetic engineering and for therapeutic development.

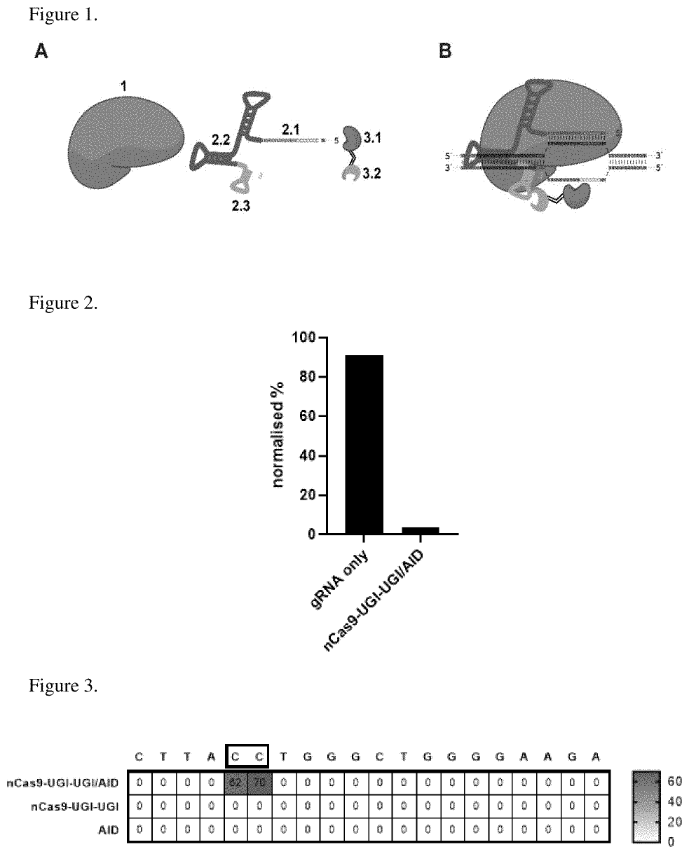

Illustrated in FIGS. 1A and 1B are schematics of an exemplary base editing system for use in the methods provided herein. The system includes three structural and functional components: (1) a sequence targeting component (e.g., a Cas protein); (2) an RNA scaffold, for sequence recognition and for effector recruitment, that comprises a crRNA, tracrRNA and an RNA motif and (3) an effector fusion protein (a non-nuclease DNA modifying enzyme such as AID fused to a small protein that binds to the RNA motif). More specifically as shown in FIG. 1A, the components of the RNA scaffold mediated base editing system for use in the methods provided herein include: a sequence targeting component 1 (such as dCas9 or nCas9_{D10A}); a chimeric RNA scaffold 2 containing a guide RNA motif 2.1 (crRNA for sequence targeting), a CRISPR motif (tracrRNA for Cas protein binding) 2.2, and a recruiting RNA aptamer motif 2.3 (RNA motif for recruiting effector-RNA binding protein fusion), and a fusion protein 3 comprising an effector 3.1 (e.g., cytidine deaminase) fused to an RNA aptamer ligand 3.2. FIG. 1B shows a schematic of the RNA scaffold mediated base editing complex at the target sequence: Cas9 binds to CRISPR RNA, the recruiting RNA aptamer recruits the effector module, forming an active complex capable of editing target C residues on the unpaired DNA within the CRISPR R-loop. The three components can be constructed in a single expression vector or in multiple separate expression vectors. The totality and the combination of the three specific components constitute the enabling of the technologic platform. Although FIG. 1B shows three components of the RNA scaffold in a particular 3′ to 5′ order, the components can also be arranged in different orders when required, such as optimization for different Cas protein variants.

As disclosed herein, there are a number of clear distinctions between recruitment mechanisms: the RNA scaffold mediated recruitment system described herein versus the direct fusion of Cas9 to effector protein system (the prior art BE system as described in WO2019/178225 (Moriarity). The modular design of the present system allows for flexible system engineering. Modules are interchangeable and many combinations of different modules can be achieved by simply swapping the nucleotide sequence of the recruiting RNA aptamer and the cognate ligand. Recruitment of an effector by direct fusion or direct interaction with the protein component of the sequence-targeting unit, on the other hand, always requires a re-engineering of a new fusion protein, which is technically more difficult with a less predictable outcome. Furthermore, RNA scaffold mediated recruitment likely facilitates oligomerization of effector proteins, while direct fusion would preclude the formation of oligomers due to steric hindrance.

Because of its relative ease of use and scalability, the CRISPR/Cas based gene system is poised to dominate the therapeutic landscape, making it an attractive gene editing technology to develop novel applications with therapeutic value. As disclosed herein, the second-generation RNA scaffold mediated base editing system takes advantages of certain aspects of the CRISPR/Cas system. To overcome the limitations associated with requirement of DSB and HDR for conventional CRISPR/Cas gene editing system, an elegant gene editing method called base editing (BE) has been developed exploiting the DNA targeting ability of Cas9 devoid of double strand cleavage activity, combined with the DNA editing capabilities of APOBEC-1, an enzyme member of the APOBEC family of DNA/RNA cytosine deaminases, see: Komor, A. C., Kim, Y. B., Packer, M. S., Zuris, J. A., Liu, D. R. Programmable editing of a target base in genomic DNA without double-stranded DNA cleavage. Nature 533(7603): 420-424 (2016). By directly fusing the deaminase effector to the to a Cas that is devoid of double strand cleavage activity, e.g. dCas9 or nCas9 protein, these tools, called base editors, can introduce targeted point mutations in genomic DNA or RNA without generating DSBs or requiring HDR activity. In essence, the BE system utilizes a CRISPR/Cas9 complex devoid of double strand cleavage activity as a DNA targeting machinery, in which the mutant Cas9 serves as an anchor to recruit cytosine or adenine deaminase through a direct protein-protein fusion.

The RNA scaffold mediated base editing as used in the methods and system provided herein takes a different approach. More specifically, in the RNA scaffold mediated base editing, the RNA component (scaffold) of the CRISPR/Cas9 complex serves as an anchor for effector recruitment by including an RNA motif (aptamer) into the RNA molecule. The RNA aptamer recruits an effector, e.g. base editing enzyme, fused to the RNA aptamer ligand. Comparing to the recruitment by direct protein fusion (prior art BE system) or other recruiting approaches by the protein component, the RNA scaffold mediated effector recruitment mechanism has a number of distinct features potentially advantageous both for system engineering and for achieving better functionality. For example, it has a modular design in which the nucleic acid sequence targeting function and effector function reside in different molecules, making it possible to independently reprogram the functional modules and to multiplex the system. It does not require re-engineering of an individual functional Cas9 fusion protein. In addition, the fusion effector is smaller in size which could potentially allow more efficient oligomerization of the functional effector. Moreover, as RNA scaffold mediated base editing does not require generation of a Cas9 fusion protein, which further increases the gene/transcription size of Cas9, RNA scaffold mediated base editing system could potentially be constructed in a way that is more efficient for packaging and delivery by viral vectors.

As demonstrated herein, the RNA scaffold mediated base editing methods and system provided herein exhibit a number of important different features compared to the previous system (first generation) described in WO2018129129 and WO2017011721 (and incorporated herein by reference in their entirety). First, the present methods and system exhibit substantially increased on-target efficacy compared to the first generation system, but still maintains low or absent detectable off-target effect. Second, the gene conversion product of the present methods and system exhibit higher purity than the first generation system. For example, in some results with first generation RNA scaffold mediated base editing, among the conversion of C at the target site, about 65% of C is converted to T, and about 35% is converted to G. By contrast, at the same target site under similar condition, a the present methods and system can produce over 90% C to T conversion. Third, the present methods and system may utilize a wide variety of cytosine deaminases from different species and different deaminase families. Many of them show clear different activity windows and preference positions from any previously described first generation constructs.

a. Sequence-Targeting Module

The sequence-targeting component of the methods and systems provided herein typically utilize a Cas protein of CRISPR/Cas systems from bacterial species as the sequence targeting protein. In some embodiments the Cas protein is from a Type II CRISPR system.

In embodiments the Cas protein is mutant Cas protein, for example, a dCas protein which contains mutations at its nuclease catalytic domains thus does not have nuclease activity, or an nCas protein which is partially mutated at one of the catalytic domains thus does not have nuclease activity for generating DSB. The Cas protein is specifically recognized by the tracrRNA component of the RNA scaffold, which guides the Cas protein to its target DNA or RNA sequence. The latter is flanked by a 3′ PAM.

Cas Proteins

Various Cas proteins may be used in this invention. A Cas protein, CRISPR-associated protein, or CRISPR protein, used interchangeably, refers to a protein of or derived from a CRISPR-Cas type I, type II, or type III system, which has an RNA-guided DNA-binding. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g. Koonin and Makarova, 2019, origins and evolution of crispr cas systems, Review Philos Trans R Soc Lond B Biol Sci. 2019 May 13; 374(1772)

In one embodiment, the Cas protein is derived from a type II CRISPR-Cas system. In exemplary embodiments, the Cas protein is or is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Legionella pneumophila, Francisella novicida, Gamma proteobacterium HTCC5015, Parasutterella excrementihominis, Sutter ella wadsworthensis, Sulfurospirillum sp. SC ADC, Ruminobacter sp. RM87, Burkholderiales bacterium 1 1 47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW. 030, Moritella sp. isolate NORP46, Fndozoicomonas sp. S-B4-1 U, Tamilnaduibacter salinus, Vibrio natriegens, Arcobacter skirrowii, Francisella philomiragia, Francisella hispaniensis, or Parendozoicomonas haliclonae.

In general, a Cas protein includes at least one RNA binding domain. The RNA binding domain interacts with the guide RNA. The Cas protein can be a wild type Cas protein or a modified version with no nuclease activity or just single-strand nicking activity. The Cas protein can be modified to increase nucleic acid binding affinity and/or specificity, alter an enzymatic activity, and/or change another property of the protein. For example, nuclease (i.e., DNase, RNase) domains of the protein can be modified, deleted, or inactivated. Alternatively, the protein can be truncated to remove domains that are not essential for the function of the protein. The protein can also be truncated or modified to optimize the activity.

In some embodiments, the Cas protein can be a mutant of a wild type Cas protein (such as Cas9) or a fragment thereof. In other embodiments, the Cas protein can be derived from a mutant Cas protein. For example, the amino acid sequence of the Cas9 protein can be modified to alter one or more properties (e.g., nuclease activity, affinity, stability, etc.) of the protein. Alternatively, domains of the Cas9 protein not involved in RNA targeting can be eliminated from the protein such that the modified Cas9 protein is smaller than the wild type Cas9 protein. In some embodiments, the present system utilizes the Cas9 protein from S. pyogenes, either as encoded in bacteria or codon-optimized for expression in mammalian cells.

A mutant Cas protein refers to a polypeptide derivative of the wild type protein, e.g., a protein having one or more point mutations, insertions, deletions, truncations, a fusion protein, or a combination thereof. The mutant has at least one of the RNA-guided DNA binding activity, or RNA-guided nuclease activity, or both. In general, the modified version is at least 50% (e.g., any number between 50% and 100%, inclusive, e.g., 50%, 60%, 70%, 75%, 80%, 85%, 90%, 95%, and 99%) identical to the wild type protein such as SEQ ID No. 20 below.

A Cas protein (as well as other protein components described in this invention) can be obtained as a recombinant polypeptide by techniques known in the art. To prepare a recombinant polypeptide, a nucleic acid encoding it can be linked to another nucleic acid encoding a fusion partner, e.g., glutathione-s-transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the proteins can be chemically synthesized (see e.g., Creighton, “Proteins: Structures and Molecular Principles,” W.H. Freeman & Co., NY, 1983), or produced by recombinant DNA technology as described herein. For additional guidance, skilled artisans may consult Frederick M. Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, N.Y., 2001).

The Cas protein described in the invention can be provided in purified or isolated form, or can be part of a composition. Preferably, where in a composition, the proteins are first purified to some extent, more preferably to a high level of purity (e.g., about 80%, 90%, 95%, or 99% or higher). Compositions according to the invention can be any type of composition desired, but typically are aqueous compositions suitable for use as, or inclusion in, a composition for RNA-guided targeting. Those of skill in the art are well aware of the various substances that can be included in such nuclease reaction compositions.

To practice the method disclosed herein for modifying a target nucleic acid, one can produce the proteins in a target cell via mRNA, protein RNA complexes (RNP), or any suitable expression vectors. Examples of expression vectors include chromosomal, non-chromosomal and synthetic DNA sequences, bacterial plasmids, minicircles, phage DNA, baculovirus, yeast plasmids, vectors derived from combinations of plasmids and phage DNA, viral DNA such as vaccinia, adenovirus, fowl pox virus, and pseudorabies. More details are described in the Expression System and Methods sections below.

As disclosed here, one can use the nuclease dead Cas9 (dCas9, for example from S. pyogenes D10A, H840A mutant protein), or the nuclease defective nickase Cas9 (nCas9, for example from S. pyogenes D10A mutant protein). dCas9 or nCas9 could also be derived from various bacterial species. Table 1 lists a non-exhausting list of examples of Cas9, and their corresponding PAM requirements. One can also use synthetic Cas substitutes such as those described in Rauch et al., Programmable RNA-Guided RNA Effector Proteins Built from Human Parts. Cell Volume 178, Issue 1, 27 Jun. 2019, Pages 122-134.e12.

TABLE 1

| Species | PAM |
|---|---|
| Streptococcus pyogenes | NGG |
| Streptococcus agalactiae | NGG |
| Staphylococcus aureus | NNGRRT |
| Streptococcus thermophiles | NNAGAAW |
| Streptococcus thermophiles | NGGNG |
| Neisseria meningitidis | NNNNGATT |
| Treponema denticola | NAAAAC |
| Other Type II CRISPR/Cas9 | |
| systems from other bacterial | |
| species | |

UGI

In some aspects of this disclosure, the above-described sequence-targeting component comprises a fusion between (a) a sequence-targeting protein, and (b) a first uracil DNA glycosylase (UNG) inhibitor peptide (UGI). For example, the fusion protein can include a Cas protein, e.g. Cas9 protein, fused to a UGI. Such fusion proteins may exhibit an increased nucleic acid editing efficiency as compared to fusion proteins not comprising an UGI domain. In some embodiments, the UGI comprises a wild type UGI sequence or one having the following amino acid sequence: spIP147391UNGI_BPPB2: Uracil-DNA glycosylase inhibitor (UGI)

(SEQ ID NO: 1)
MTNLSDIIEKETGKQLVIQESILMLPEEVEEVIGNKPESDILVHTAYDES
TDENVMLLTSDAPEYKPWALVIQDSNGENKIKML.

In some embodiments, the UGI proteins provided herein include fragments of UGI and proteins homologous to a UGI or a UGI fragment. For example, in some embodiments, a UGI comprises a fragment of the amino acid sequence set forth above. In some embodiments, a UGI comprises an amino acid sequence homologous to the amino acid sequence set forth above or an amino acid sequence homologous to a fragment of the amino acid sequence set forth in the UGI sequence above. In some embodiments, proteins comprising UGI or fragments of UGI or homologs of UGI or UGI fragments are referred to as “UGI variants.” A UGI variant shares homology to UGI, or a fragment thereof. For example, a UGI variant is at least about 70% (e.g., at least about 80%, 90%, 95%, 96%, 97%, 98%, 99%) to a wild type UGI or the UGI sequence as set forth above.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J Mol. Biol. 287:331-346(1999), the entire contents of each are incorporated herein by reference.

b. RNA Scaffold for Sequence Recognition and Effector Recruitment:

The second component of the platform disclosed herein is an RNA scaffold, which has three sub-components: a crRNA comprising a programmable guide RNA sequence, a tracrRNA, and a RNA motif. This scaffold can be either a single RNA molecule or a complex of multiple RNA molecules. As disclosed herein, the crRNA, tracrRNA and the Cas protein together form a CRISPR/Cas-based module for sequence targeting and recognition, while the RNA motif recruits, via an RNA-protein binding pair, a protein effector, such as a base editing enzyme, which carries out the genetic modification. Accordingly, the RNA scaffold connects the effector module (e.g. base editing enzyme) and sequence recognition module (e.g. Type II Cas protein).

Programmable Guide RNA (crRNA)

One key sub-component is the programmable guide RNA. Due to its simplicity and efficiency, the CRISPR-Cas system has been used to perform genome-editing in cells of various organisms. The specificity of this system is dictated by base pairing between a target DNA and a custom-designed guide RNA. By engineering and adjusting the base-pairing properties of guide RNAs, one can target any sequences of interest provided that there is a PAM sequence in a target sequence.

Among the sub-components of the RNA scaffold disclosed herein, the guide sequence provides the targeting specificity. It includes a region that is complementary and capable of hybridization to a pre-selected target site of interest. In various embodiments, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In an exemplary embodiment, the guide sequence is about 17-20 nucleotides in length, such as 20 nucleotides.

One requirement for selecting a suitable target nucleic acid is that it has a 3′ PAM site/sequence. Each target sequence and its corresponding PAM site/sequence are referred herein as a Cas-targeted site. The Type II CRISPR system, one of the most well characterized systems, needs only Cas9 protein and a guide RNA complementary to a target sequence to affect target cleavage. The type II CRISPR system of S. pyogenes uses target sites having N12-20NGG, where NGG represents the PAM site from S. pyogenes, and N12-20 represents the 12-20 nucleotides directly 5′ to the PAM site. Additional PAM site sequences from other species of bacteria include NGGNG, NNNNGATT, NNAGAA, NNAGAAW, and NAAAAC. See, e.g., US 20140273233, WO 2013176772, Cong et al., (2012), Science 339 (6121): 819-823, Jinek et al., (2012), Science 337 (6096): 816-821, Mali et al, (2013), Science 339 (6121): 823-826, Gasiunas et al., (2012), Proc Natl Acad Sci USA. 109 (39): E2579-E2586, Cho et al., (2013) Nature Biotechnology 31, 230-232, Hou et al., Proc Natl Acad Sci USA. 2013 Sep. 24; 110(39):15644-9, Mojica et al., Microbiology. 2009 March; 155(Pt 3):733-40, and www.addgene.org/CRISPR/. The contents of these documents are incorporated herein by reference in their entireties.

The target nucleic acid strand can be either of the two strands on a genomic DNA in a host cell. Examples of such genomic dsDNA include, but are not necessarily limited to, a host cell chromosome, mitochondrial DNA and a stably maintained plasmid. However, it is to be understood that the present method can be practiced on other dsDNA present in a host cell, such as non-stable plasmid DNA, viral DNA, and phagemid DNA, as long as there is Cas-targeted site regardless of the nature of the host cell dsDNA. The present method can be practiced on RNAs too.

tracrRNA

Besides the above-described guide sequence, the RNA scaffold of this invention includes a tracrRNA. For example, the scaffold can be a hybrid RNA molecule where the above-described programmable guide RNA is fused to a tracrRNA to mimic the natural crRNA:tracrRNA duplex. Shown below is an exemplary hybrid crRNA:tracrRNA, gRNA sequence: 5′-(20nt guide)-GUUUAAGAGCUAUGCUGGAAACAGCAUAGCAAGUUUAAAUAAGGCUAGUCCGUUAUCAACUUGA AAAAGUGGCACCGAGUCGGUGCUUUUUUU-3′ (SEQ ID No: 2; Chen et al. Cell 2013 Dec. 19; 155(7):1479-91). Various tracrRNA sequences are known in the art and examples include the following tracrRNAs and active portions thereof. As used herein, an active portion of a tracrRNA retains the ability to form a complex with a Cas protein, such as Cas9 or dCas9 or nCas9. See, e.g., WO2014144592. Methods for generating crRNA-tracrRNA hybrid RNAs (also known as single guide RNAs or sgRNAs) are known in the art. In embodiments where the crRNA and tracrRNA are provided as a single gRNA (sgRNA) the two components may be linked together via a tetra loop. See e.g., WO2014099750, US 20140179006, and US 20140273226. The contents of these documents are incorporated herein by reference in their entireties.

(SEQ ID No: 3)
GGAACCAUUCAAAACAGCAUAGCAAGUUAAAAUAAGGCUAGUCCGUUAUC
AACUUGAAAAAGUGGCACCGAGUCGGUGC;
(SEQ ID No: 4)
UAGCAAGUUAAAAUAAGGCUAGUCCGUUAUCAACUUGAAAAAGUGGCACC
GAGUCGGUGC;
(SEQ ID No: 5)
AGCAUAGCAAGUUAAAAUAAGGCUAGUCCGUUAUCAACUUGAAAAAGUGG
CACCGAGUCGGUGC;
(SEQ ID No: 6)
CAAAACAGCAUAGCAAGUUAAAAUAAGGCUAGUCCGUUAUCAACUUGAAA
AAGUGGCACCGAGUCGGUGC;
(SEQ ID No: 7)
UAGCAAGUUAAAAUAAGGCUAGUCCGUUAUCAACUUGAAAAAGUG;
(SEQ ID No: 8)
UAGCAAGUUAAAAUAAGGCUAGUCCGUUAUCA;
and
(SEQ ID No: 9)
UAGCAAGUUAAAAUAAGGCUAGUCCG.

In some embodiments the tracrRNA is from Strep pyogenes.

In some embodiments, the tracrRNA activity and the crRNA comprising the guide sequence are two separate RNA molecules, which together form the functional guide RNA and part of the RNA scaffold. In this case, the molecule with the tracrRNA activity should be able to interact with (usually by base pairing) the molecule (crRNA) having the guide sequence to form a two part guide crRNA:tracRNA.

Recruiting RNA Motif

The third sub-component of the RNA scaffold is the RNA motif(s), which, in effect, recruits the effector protein (base editing enzyme) to the target DNA. This linkage is critical for the base editing systems and methods disclosed herein.

A prior art method to recruit effector/DNA editing enzymes to a target sequence is through a direct fusion of an effector protein to dCas9. The direct fusion of effector enzymes to the proteins required for sequence recognition (such as dCas9) has achieved success in sequence specific transcriptional activation or suppression, but the protein-protein fusion design may render spatial hindrance, which is not ideal for enzymes that need to form a multimeric complex for their activities. In fact, most nucleotide editing enzymes (such as AID or APOBEC3G) require formation of dimers, tetramers or higher order oligomers, for their DNA editing catalytic activities. The direct fusion to dCas9, which anchors to DNA in a defined conformation, would hinder the formation of a functional oligomeric enzyme complex at the right location.

In contrast, the base editing systems and methods provided herein are based on RNA scaffold-mediated effector protein recruitment. More specifically, the platform takes advantage of various RNA motif/RNA binding protein binding pairs. To this end, an RNA scaffold is designed such that an RNA motif (e.g., MS2 operator motif), which specifically binds to an RNA binding protein (e.g., MS2 coat protein, MCP), is linked to the gRNA-CRISPR scaffold.

As a result, this RNA scaffold component of the platform disclosed herein is a designed RNA molecule, which contains not only the gRNA motif (crRNA) for specific DNA/RNA sequence recognition, the CRISPR RNA motif (tracrRNA) for Cas protein binding, but also the RNA motif for effector recruitment (FIG. 1B). In this way, recruited-effector protein fusions can be recruited to the target site through their ability to bind to the RNA motif. Due to the flexibility of RNA scaffold mediated recruitment, a functional monomer, as well as dimer, tetramer, or oligomer could be relatively easy to form near the target DNA or RNA sequence. These pairs of RNA motif/binding protein could be derived from naturally occurring sources (e.g., RNA phages, or yeast telomerase) or could be artificially designed (e.g., RNA aptamers and their corresponding binding protein ligands). A non-exhaustive list of examples of recruiting RNA motif/RNA binding protein pairs that could be used in the methods and systems provided herein is summarized in Table 2. As will be apparent to the skilled person, chemically modified versions and/or or sequence variants of the aptamers and their binding partners may also be utilized.

TABLE 2
Examples of recruiting RNA motifs that can be used in this invention,
as well as their pairing RNA binding proteins/protein domains.

| RNA motif | Pairing interacting protein* | Organism |
|---|---|---|
| Telomerase Ku binding motif | Ku | Yeast |
| Telomerase Sm7 binding motif | Sm7 | Yeast |
| MS2 phage operator stem- | MS2 Coat Protein (MCP) | Phage |
| loop | | |
| PP7 phage operator stem-loop | PP7 coat protein (PCP) | Phage |
| SfMu phage Com stem-loop | Com RNA binding protein | Phage |
| Non-natural RNA aptamer | Corresponding aptamer ligand | Artificially |
| | | designed |

*Recruited proteins are fused to effector proteins, for examples see Table 3.

The sequences for the above binding pairs are listed below.

1. Telomerase Ku Biding Motif/Ku Heterodimer

a. Ku Binding Hairpin

(SEQ ID No: 10)
5′-UUCUUGUCGUACUUAUAGAUCGCUACGUUAUUUCAAUUUUGAAAAUC
UGAGUCCUGGGAGUGCGGA-3′

b. Ku Heterodimer

(SEQ ID No: 11)
MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD>
MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISERHSIHWPCRLTIGSNLSIRIAAYKSILQERVKKTWTVVD
AKTLKKEDIQKETVYCLNDDDETEVLKEDIIQGFRYGSDIVPFSKVDEEQ
MKYKSEGKCFSVLGFCKSSQVQRRFFMGNQVLKVFAARDDEAAAVALSSL
IHALDDLDMVAIVRYAYDKRANPQVGVAFPHIKHNYECLVYVQLPFMEDL
RQYMFSSLKNSKKYAPTEAQLNAVDALIDSMSLAKKDEKTDTLEDLFPTT
KIPNPRFQRLFQCLLHRALHPREPLPPIQQHIWNMLNPPAEVTTKSQIPL
SKIKTLEPLIEAKKKDQVTAQEIFQDNHEDGPTAK

2. Telomerase Sm7 Biding Motif/Sm7 Homoheptamer

a. Sm Consensus Site (Single Stranded)

(SEQ ID No: 12)
5′-AAUUUUUGGA-3′

b. Monomeric Sm—Like Protein (Archaea)

(SEQ ID No: 13)
GSVIDVSSQRVNVQRPLDALGNSLNSPVIIKLKGDREFRGVLKSFDLHMN
LVLNDAEELEDGEVTRRLGTVLIRGDNIVYISP

3. MS2 Phage Operator Stem Loop/MS2 Coat Protein

a. MS2 Phage Operator Stem Loop

(SEQ ID No: 14)
5′- GCGCACAUGAGGAUCACCCAUGUGC -3′

b. MS2 Coat Protein

(SEQ ID No: 15)
MASNFTQFVLVDNGGTGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY

4. PP7 Phage Operator Stem Loop/PP7 Coat Protein

a. PP7 Phage Operator Stem Loop

(SEQ ID No: 16)
5′-aUAAGGAGUUUAUAUGGAAACCCUUA -3′

b. PP7 Coat Protein (PCP)

(SEQ ID No: 17)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVATSQVEDLVVNLVPLGR.

5. SfMu Com Stem Loop/SfMu Com Binding Protein

a. SfMu Com Stem Loop

(SEQ ID No: 18)
5′-CUGAAUGCCUGCGAGCAUC-3′

b. SfMu Com Binding Protein

(SEQ ID No: 19)
MKSIRCKNCNKLLFKADSFDHIEIRCPRCKRHIIMLNACEHPTEKHCGKR
EKITHSDETVRY

The RNA scaffold can be either a single RNA molecule or a complex of multiple RNA molecules. For example, the guide RNA, tracrRNA, and recruiting RNA motif can be three segments of one, long single RNA molecule. Alternatively, one, two or three of them can be on separate molecules. In the latter case, the three components can be linked together to form the scaffold via covalent or non-covalent linkage or binding, including e.g., Watson-Crick base-pairing.

In one example, the RNA scaffold can comprise two separate RNA molecules. The first RNA molecule can comprise the programmable guide RNA and a region that can form a stem duplex structure with a complementary region. The second RNA molecule can comprise the complementary region in addition to the tracrRNA and the RNA motif. Via this stem duplex structure, the first and second RNA molecules form an RNA scaffold of this invention. In one embodiment, the first and second RNA molecules each comprise a sequence (of about 6 to about 20 nucleotides) that base pairs to the other sequence. By the same token, the tracrRNA and the RNA motif can also be on different RNA molecules and be brought together with another stem duplex structure.

The RNAs and related scaffold of this invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U).

The Cas protein-guide RNA scaffold complexes can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties. The complexes can be isolated or purified, at least to some extent, from cellular material of a cell or an in vitro translation-transcription system in which they are produced.

Modifications

The RNA scaffold as disclosed herein may include one or more modifications. Such modifications may include inclusion of at least one non-naturally occurring nucleotide, or a modified nucleotide, or analogues thereof. Examples of such modifications include but are not limited to the addition of nucleotides to extend sequences, substitution of nucleotides, addition of linker sequences and modifying the positioning of various components of the RNA scaffold.

Nucleotides may be modified at the ribose, phosphate linkage, and/or base moiety. Modified nucleotides may include 2′-O-methyl analogs, 2′-fluoro analogs or 2′-deoxy analogs or 2′-ribose analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, 5-methylcytidine, 5-methoxyuridine, pseudouridine, inosine, 7-methylguanosine. These modifications may apply to any component of the RNA scaffold. These modifications may apply to any component of the CRISPR system. In a preferred embodiment these modifications are made to the RNA components, e.g., the guide RNA sequence.

In some embodiments, the RNA scaffold described above or a subsection thereof can comprise one or more modifications, e.g., a base modification, a backbone modification, etc, to provide the nucleic acid with a new or enhanced feature (e.g., improved stability).

Modified Backbones and Modified Inter-Nucleoside Linkages

Examples of suitable nucleic acids containing modifications include nucleic acids containing modified backbones, bases, sugars, or non-natural internucleoside linkages. Nucleic acids (having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH2-NH—O—CH2-, —CH2-N(CH3)-O—CH2-(known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-). MMI type internucleoside linkages are disclosed in the above referenced U.S. Pat. No. 5,489,677. Suitable amide internucleoside linkages are disclosed in U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Modified Sugar Moieties

A subject nucleic acid can also include one or more substituted sugar moieties. Suitable polynucleotides comprise a sugar substituent group selected from: OH; H; F; O-, S-, or N-alkyl; O-, S-, or N-alkenyl; O-, S- or N-alkynyl; or O-alkyl-Co-alkyl, wherein the alkyl, alkenyl and alkynyl may be substituted or unsubstituted C1 to C10 alkyl or C2 to C10 alkenyl and alkynyl. Particularly suitable are O((CH2)nO)mCH3, O(CH2)nOCH3, O(CH2)nNH2, O(CH2)nCH3, O(CH2)nONH2, and O(CH2)nON((CH2)nCH3)₂, where n and m are from 1 to about 10. Other suitable polynucleotides comprise a sugar substituent group selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH2 CH2OCH3, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH2)2ON(CH3)2 group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O-CH2-O-CH2-N(CH3)2.

Other suitable sugar substituent groups include methoxy (—O—CH3), aminopropoxy (—OCH2 CH2 CH2NH2), allyl (—CH2-CH═CH2), —O-allyl CH2-CH═CH2) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Modifications as disclosed herein can be incorporated at various positions of the RNA scaffold such as at the tetra loop of a sgRNA, at the 3′ end of the CRISPR motif and at the RNA motif, in stem loop 1, 2 or 3. Modifications disclosed herein include an extension of the repeat anti-repeat of a sgRNA, positioning of the RNA motif at the 3′ end of the CRISPR motif, linker sequence linking the RNA motif to the CRISPR motif, modifying the RNA motifs nucleotides and extending the RNA motif.

### Positioning of the RNA Motif

The RNA motif may be positioned at various positions of the RNA scaffold as described in Example 1. The RNA motif e.g. the MS2 aptamer can be positioned at the 3′ end of the CRISPR motif, at the tetra loop of the sgRNA, at stem loop 2 of the CRISPR motif and at the stem loop 3 of the CRISPR motif. The positioning of the MS2 aptamer is crucial due to the steric hindrance that can result from the bulky loops. In a preferred embodiment, the MS2 aptamer is at the 3′ end of the CRISPR motif. Advantageously, the positioning of the MS2 aptamer at the 3′ end of the CRISPR motif is in space therefore reducing steric hindrance with other bulky loops of the RNA scaffold.

### Linker Sequence

The RNA motif may be linked to the CRISPR motif via a linker sequence. The linker sequence may be 2, 3, 4, 5, 6, 7 or more than 7 nucleotides. Advantageously, the linker sequence provides flexibility to the RNA scaffold. The linker sequence may be a GC rich sequence.

### Modifying the RNA Motif's Nucleotides

Modifications may be made to the RNA motif e.g. aptamer sequence. For example a suitable modification is to the C-5 and F-5 aptamer mutant. In a preferred embodiment, the modification to the aptamer is a substitution of the adenine to 2-aminopurine (2-AP) at position 10. Advantageously, the substitution induces conformational changes resulting in greater affinity. The affinity for the MS2 coat protein by the F-5 mutant was approximately 3-fold higher than the parental F5 sequence. Whilst not wishing to be bound by any theory, it is believed that the conformational change induced by 2-AP results in hydrogen bond formation between the exocyclic amino group of the 2-AP nucleotide at position 10 and the carbonyl the B59 at the backbone. It is thought that replacing the MS2 hairpin sequence with the higher affinity MS2 sequences will result in increased based editing efficiencies because substituting amino acids helps to order the RNA stem loop into a conformation that is better recognised by the coat protein.

Suitable modifications to the RNA motif are listed above, such as 2′ deoxy-2-aminopurine, 2′ribose-2-aminopurine, phosphorothioate mods, 2′-Omethyl mods, 2′-Fluro mods and LNA mods. Advantageously, the modifications help to increase stability and promote stronger bonds/folding structure of the desired hairpin.

### Extension of RNA Motif

The length of the RNA motif extension can be variable. The extension sequence of the RNA motif is a double-stranded extension, wherein the extension sequence of the RNA motif comprises 2-24 nucleotides. In one embodiment, a 4 nucleotide extension results in the stem having 23 nucleotides in total length. In another embodiment, a 10 nucleotide extension results in the stem having 29 nucleotides in total length. In another embodiment, a 16 nucleotide extension results in the stem having 35 nucleotides in total length. In another embodiment, a 26 nucleotide extension results in the stem having 45 nucleotides in total length. The extension to the RNA motif can be more than 24 nucleotides. Advantageously, the extension of the RNA motif increases flexibility of the motif. The extension to the RNA motif may be a double-stranded or a single-stranded extension. Double-stranded extension provides greater stabilization of the RNA scaffold.

### Extension of the Repeat: Anti-Repeat Upper Stem

The nucleic acid-targeting motif comprising a guide RNA sequence (crRNA) and CRISPR RNA motif (tracrRNA) can be provided as a sgRNA. The two components are linked via a repeat: anti-repeat. The repeat: anti-repeat upper stem can be extended to increase the flexibility, proper folding and stability of the loop. There is an extension of 2, 3, 4, 5, 6, 7 bases or more than 7 bases at either side of the repeat: anti-repeat region. In a preferred embodiment, the repeat: anti-repeat region has an extension of 7 nucleotides at either side of the upper stem. The extension of 7 bases at either side of the upper stem results in a region that is 14 base pairs longer. The 7 base extension at either side of the upper stem results in the upper stem having a total of 11 bases at either side and a total length of 22 nucleotides when the RNA scaffold is synthesized as one single RNA molecule. The 7 base extension at either side of the upper stem results in the upper stem having a total of 11 bases at either side and a total length of 25 nucleotides when the RNA scaffold is synthesized as two separate RNA molecule.

### Combination of Modifications

The RNA scaffold may have one or more of the above-mentioned modifications. The one or more modification can be on the different components of the RNA scaffold e.g. extension of tetra loop of the sgRNA and extension of the RNA motif, or can be on the same component of the RNA scaffold, e.g. extension of the RNA motif and substitution of the RNA motifs nucleotides. The modifications may be two or more, three or more, four or more, or five or more. In one embodiment, the modification may be the extension of the RNA motif and/or may the substitution of one or more nucleotides

An example of an RNA motif as used herein is the MS2 aptamer. The MS2 motif specifically binds to the MS2 bacteriophage coat protein (MCP). The RNA motif may be a MS2 C-5 mutant or a MS2 F-5 mutant. One of the significant differences between the wild-type MS2 and the C-5 and F-5 mutants is the substitution of the Uracil nucleotide to Cytosine at position 5 of the aptamer loop. The F-5 mutant has been reported to have higher affinity for the coat protein compared to the wild-type and other members of the aptamer family. Suitably, both C-5 mutants and F-5 mutants are used as aptamers in the present invention. In one embodiment, the MS2 aptamer is a wild-type MS2, a mutant MS2 or variants thereof. In another embodiment, the MS2 aptamer comprises a C-5 and/or F-5 mutation. The MS2 protein linked to the CRISPR motif can be a single-copy (i.e. one MS2 loop) or a double-copy (i.e. two MS2 loops). In a preferred embodiment, the RNA motif is a single-copy. In other embodiments, the RNA motif is more than one copy.

c. Effectors: Non-Nuclease DNA Modifying Enzymes

The third component of the platform disclosed in this invention is a non-nuclease effector. The effector is not a nuclease and does not have any nuclease activity but can have the activity of other types of DNA modifying enzymes, for example base editing. Examples of the enzymatic activity include, but are not limited to, deamination activity, methyltransferase activity, demethylase activity, DNA repair activity, DNA damage activity, dismutase activity, nickase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, the effector has the activity of cytidine deaminases (e.g., AID, APOBEC3G), adenosine deaminases (e.g., ADA and tadA), DNA methyltransferases, and DNA demethylases. In some embodiments the effector is modified to induce or improve DNA editing activity, for example in the case of ADA and tadA which require modification to edit DNA. In some embodiments, the effectors are from different vertebrate animal species and have distinct properties, or could be novel chimeric proteins assembled in silico to bring together the desirable attributes and activities.

In preferred embodiments, this third component is a conjugate or a fusion protein that has an RNA-binding domain and an effector domain. These two domains can be joined via a linker.

In some embodiments, no effector is needed in some cell types (e.g., cancer lines over-expressing deaminases). In that case, endogenous effector (e.g. APOBEC, AID, etc) can be gene-edited to include the recruitment module, so no exogenous editor is needed. This is applicable to cell types that express the editor of interest—e.g., lymphoid (B+T cells) and certain cancer cells. In addition, the nickase activity does not have to come from the Cas module but can be recruited from the effectors—for example, dCas9 can have an aptamer to recruit both the nickase and editor via the same gRNA recruitment.

RNA-Binding Domain

Although various RNA-binding domains can be used in this invention, the RNA-binding domain of Cas protein (such as Cas9) or its variant (such as dCas9) should not be used. As mentioned above, the direct fusion to dCas9, which anchors to DNA in a defined conformation, would hinder the formation of a functional oligomeric enzyme complex at the right location. Instead, the present invention takes advantages of various other RNA motif-RNA binding protein binding pairs. Examples include those listed in Table 2.

In this way, the effector protein can be recruited to the target site through RNA-binding domain's ability to bind to the recruiting RNA motif. Due to the flexibility of RNA scaffold mediated recruitment, a functional monomer, as well as dimer, tetramer, or oligomer could be formed relatively easily near the target DNA or RNA sequence.

Effector Domain

The effector component comprises an activity portion, i.e., an effector domain. In some embodiments, the effector domain comprises the naturally occurring activity portion of a non-nuclease protein (e.g., deaminases). In other embodiments, the effector domain comprises a modified amino acid sequence (e.g., substitution, deletion, insertion) of a naturally occurring activity portion of a non-nuclease protein. The effector domain has an enzymatic activity. Examples of this activity include deamination activity, methyltransferase activity, demethylase activity, DNA repair activity, DNA damage activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, DNA methylation, histone acetylation activity, or histone methylation activity. Some modifications in non-nuclease protein (e.g., deaminases) can help reduce off-target effect. For example, as described below, one can reduce the recruitment of AID to off-target sites by mutating Ser38 in AID to Ala (SEQ ID NO:29).

Linker

The above-mentioned two domains as well as others as disclosed herein can be joined by means of linkers, such as, but not limited to chemical modification, peptide linkers, chemical linkers, covalent or non-covalent bonds, or protein fusion or by any means known to one skilled in the art. The joining can be permanent or reversible. See for example U.S. Pat. Nos. 4,625,014, 5,057,301 and 5,514,363, US Application Nos. 20150182596 and 20100063258, and WO2012142515, the contents of which are incorporated herein in their entirety by reference. In some embodiments, several linkers can be included in order to take advantage of desired properties of each linker and each protein domain in the conjugate. For example, flexible linkers and linkers that increase the solubility of the conjugates are contemplated for use alone or with other linkers. Peptide linkers can be linked by expressing DNA encoding the linker to one or more protein domains in the conjugate. Linkers can be acid cleavable, photocleavable and heat sensitive linkers. Methods for conjugation are well known by persons skilled in the art and are encompassed for use in the present invention.

In some embodiments, the RNA-binding domain and the effector domain can be joined by a peptide linker. Peptide linkers can be linked by expressing nucleic acid encoding in frame the two domains and the linker Optionally the linker peptide can be joined at either or both of the amino terminus and carboxy terminus of the domains. In some examples, a linker is an immunoglobulin hinge region linker as disclosed in U.S. Pat. Nos. 6,165,476, 5,856,456, US Application Nos. 20150182596 and 2010/0063258 and International Application WO2012/142515, each of which are incorporated herein in their entirety by reference.

Other Domains

The effector fusion protein can comprise other domains. In certain embodiments, the effector fusion protein can comprise at least one nuclear localization signal (NLS). In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

In some embodiments, the fusion protein can comprise at least one cell-penetrating domain to facilitate delivery of the protein into a target cell. In one embodiment, the cell-penetrating domain can be a cell-penetrating peptide sequence. Various cell-penetrating peptide sequences are known in the art and examples include that of the HIV-1 TAT protein, TLM of the human HBV, Pep-1, VP22, and a polyarginine peptide sequence.

In still other embodiments, the fusion protein can comprise at least one marker domain. Non-limiting examples of marker domains include fluorescent proteins, purification tags, and epitope tags. In some embodiments, the marker domain can be a fluorescent protein. In other embodiments, the marker domain can be a purification tag and/or an epitope tag. See, e.g., US 20140273233.

In one embodiment, AID was used as an example to illustrate how the system works. AID is a cytidine deaminase that can catalyze the reaction of deamination of cytidine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. As excision repair pathway is present in most, if not all, somatic cells, recruitment of AID to the target site can correct a C-G base pair to others. In that case, if a C-G base pair is an underlying disease-causing genetic mutation in somatic tissues/cells, the above-described approach can be used to correct the mutation and thereby treat the disease.

By the same token, if an underlying disease causing genetic mutation is an A-T base pair at a specific site, one can use the same approach to recruit an adenosine deaminase to the specific site, where adenosine deaminase can correct the A-T base pair to others, see for example David Liu—U.S. Ser. No. 10/113,163. Other effector enzymes are expected to generate other types of changes in base-pairing. A non-exhaustive list of examples of DNA/RNA modifying enzymes is detailed in Table 3.

TABLE 3
Examples of effector proteins that can be used in this invention

| | Genetic | Effector protein |
|---|---|---|
| Enzyme type | change | abbreviated |
| Cytidine | C→U/T | AID |
| deaminase | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| | | CDA |
| Adenosine | A→I/G | ADA |
| deaminase | | ADAR1 |
| | | ADAR2 |
| | | ADAR3 |
| | | tadA |
| DNA Methyl | C→Met-C | Dnmt1 |
| transferase | | |
| | | Dnmt3b |
| Demethylase | Met-C→ C | Tet1 |
| | | Tet2 |
| | | TDG |

Effector protein full names:
AID: activation induced cytidine deaminase, a.k.a AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
ADA: adenosine deaminase
ADAR1: adenosine deaminase acting on RNA 1
ADAR2: adenosine deaminase acting on RNA 2
ADAR3: adenosine deaminase acting on RNA 3
CDA: Cytidine deaminase
Dnmt1: DNA (cytosine-5-)-methyltransferase 1
Dnmt3a: DNA (cytosine-5-)-methyltransferase 3 alpha
Dnmt3b: DNA (cytosine-5-)-methyltransferase 3 beta
tadA: tRNA adenosine deaminase
Tet1:ten-eleven translocation 1
Tet2: ten-eleven translocation 2
Tdg: thymine DNA glycosylase

The above-described three specific components constitute the technological platform. Each component could be chosen from the list in Table 1-3 respectively to achieve a specific therapeutic/utility goal.

In one example, an RNA scaffold mediated recruitment system was constructed using (i) dCas9 from S. pyogenes as the sequence targeting protein, (ii) an RNA scaffold containing a guide RNA sequence, a CRISPR RNA motif, and a MS2 operator motif, and (iii) an effector fusion containing a human AID fusing to MS2 operator binding protein MCP. The sequences for the components are listed below

S. pyogenes dCas9 protein sequence (SEQ ID No. 20)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD
(Residues underlined: D10A, H840A active site mutants)
Cas9 D10A Protein (residues underlined: D10A, SEQ ID No: 21)
DKKYSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD
DNA encoding Cas9 D10A Protein (SEQ ID No: 22)
GATAAAAAGTATTCTATTGGTTTAGCCATCGGCACTAATTCCGTTGGATGGGCTGTCATAACCG
ATGAATACAAAGTACCTTCAAAGAAATTTAAGGTGTTGGGGAACACAGACCGTCATTCGATTAA
AAAGAATCTTATCGGTGCCCTCCTATTCGATAGTGGCGAAACGGCAGAGGCGACTCGCCTGAAA
CGAACCGCTCGGAGAAGGTATACACGTCGCAAGAACCGAATATGTTACTTACAAGAAATTTTTA
GCAATGAGATGGCCAAAGTTGACGATTCTTTCTTTCACCGTTTGGAAGAGTCCTTCCTTGTCGA
AGAGGACAAGAAACATGAACGGCACCCCATCTTTGGAAACATAGTAGATGAGGTGGCATATCAT
GAAAAGTACCCAACGATTTATCACCTCAGAAAAAAGCTAGTTGACTCAACTGATAAAGCGGACC
TGAGGTTAATCTACTTGGCTCTTGCCCATATGATAAAGTTCCGTGGGCACTTTCTCATTGAGGG
TGATCTAAATCCGGACAACTCGGATGTCGACAAACTGTTCATCCAGTTAGTACAAACCTATAAT
CAGTTGTTTGAAGAGAACCCTATAAATGCAAGTGGCGTGGATGCGAAGGCTATTCTTAGCGCCC
GCCTCTCTAAATCCCGACGGCTAGAAAACCTGATCGCACAATTACCCGGAGAGAAGAAAAATGG
GTTGTTCGGTAACCTTATAGCGCTCTCACTAGGCCTGACACCAAATTTTAAGTCGAACTTCGAC
TTAGCTGAAGATGCCAAATTGCAGCTTAGTAAGGACACGTACGATGACGATCTCGACAATCTAC
TGGCACAAATTGGAGATCAGTATGCGGACTTATTTTTGGCTGCCAAAAACCTTAGCGATGCAAT
CCTCCTATCTGACATACTGAGAGTTAATACTGAGATTACCAAGGCGCCGTTATCCGCTTCAATG
ATCAAAAGGTACGATGAACATCACCAAGACTTGACACTTCTCAAGGCCCTAGTCCGTCAGCAAC
TGCCTGAGAAATATAAGGAAATATTCTTTGATCAGTCGAAAAACGGGTACGCAGGTTATATTGA
CGGCGGAGCGAGTCAAGAGGAATTCTACAAGTTTATCAAACCCATATTAGAGAAGATGGATGGG
ACGGAAGAGTTGCTTGTAAAACTCAATCGCGAAGATCTACTGCGAAAGCAGCGGACTTTCGACA
ACGGTAGCATTCCACATCAAATCCACTTAGGCGAATTGCATGCTATACTTAGAAGGCAGGAGGA
TTTTTATCCGTTCCTCAAAGACAATCGTGAAAAGATTGAGAAAATCCTAACCTTTCGCATACCT
TACTATGTGGGACCCCTGGCCCGAGGGAACTCTCGGTTCGCATGGATGACAAGAAAGTCCGAAG
AAACGATTACTCCATGGAATTTTGAGGAAGTTGTCGATAAAGGTGCGTCAGCTCAATCGTTCAT
CGAGAGGATGACCAACTTTGACAAGAATTTACCGAACGAAAAAGTATTGCCTAAGCACAGTTTA
CTTTACGAGTATTTCACAGTGTACAATGAACTCACGAAAGTTAAGTATGTCACTGAGGGCATGC
GTAAACCCGCCTTTCTAAGCGGAGAACAGAAGAAAGCAATAGTAGATCTGTTATTCAAGACCAA
CCGCAAAGTGACAGTTAAGCAATTGAAAGAGGACTACTTTAAGAAAATTGAATGCTTCGATTCT
GTCGAGATCTCCGGGGTAGAAGATCGATTTAATGCGTCACTTGGTACGTATCATGACCTCCTAA
AGATAATTAAAGATAAGGACTTCCTGGATAACGAAGAGAATGAAGATATCTTAGAAGATATAGT
GTTGACTCTTACCCTCTTTGAAGATCGGGAAATGATTGAGGAAAGACTAAAAACATACGCTCAC
CTGTTCGACGATAAGGTTATGAAACAGTTAAAGAGGCGTCGCTATACGGGCTGGGGACGATTGT
CGCGGAAACTTATCAACGGGATAAGAGACAAGCAAAGTGGTAAAACTATTCTCGATTTTCTAAA
GAGCGACGGCTTCGCCAATAGGAACTTTATGCAGCTGATCCATGATGACTCTTTAACCTTCAAA
GAGGATATACAAAAGGCACAGGTTTCCGGACAAGGGGACTCATTGCACGAACATATTGCGAATC
TTGCTGGTTCGCCAGCCATCAAAAAGGGCATACTCCAGACAGTCAAAGTAGTGGATGAGCTAGT
TAAGGTCATGGGACGTCACAAACCGGAAAACATTGTAATCGAGATGGCACGCGAAAATCAAACG
ACTCAGAAGGGGCAAAAAAACAGTCGAGAGCGGATGAAGAGAATAGAAGAGGGTATTAAAGAAC
TGGGCAGCCAGATCTTAAAGGAGCATCCTGTGGAAAATACCCAATTGCAGAACGAGAAACTTTA
CCTCTATTACCTACAAAATGGAAGGGACATGTATGTTGATCAGGAACTGGACATAAACCGTTTA
TCTGATTACGACGTCGATCACATTGTACCCCAATCCTTTTTGAAGGACGATTCAATCGACAATA
AAGTGCTTACACGCTCGGATAAGAACCGAGGGAAAAGTGACAATGTTCCAAGCGAGGAAGTCGT
AAAGAAAATGAAGAACTATTGGCGGCAGCTCCTAAATGCGAAACTGATAACGCAAAGAAAGTTC
GATAACTTAACTAAAGCTGAGAGGGGTGGCTTGTCTGAACTTGACAAGGCCGGATTTATTAAAC
GTCAGCTCGTGGAAACCCGCCAAATCACAAAGCATGTTGCACAGATACTAGATTCCCGAATGAA
TACGAAATACGACGAGAACGATAAGCTGATTCGGGAAGTCAAAGTAATCACTTTAAAGTCAAAA
TTGGTGTCGGACTTCAGAAAGGATTTTCAATTCTATAAAGTTAGGGAGATAAATAACTACCACC
ATGCGCACGACGCTTATCTTAATGCCGTCGTAGGGACCGCACTCATTAAGAAATACCCGAAGCT
AGAAAGTGAGTTTGTGTATGGTGATTACAAAGTTTATGACGTCCGTAAGATGATCGCGAAAAGC
GAACAGGAGATAGGCAAGGCTACAGCCAAATACTTCTTTTATTCTAACATTATGAATTTCTTTA
AGACGGAAATCACTCTGGCAAACGGAGAGATACGCAAACGACCTTTAATTGAAACCAATGGGGA
GACAGGTGAAATCGTATGGGATAAGGGCCGGGACTTCGCGACGGTGAGAAAAGTTTTGTCCATG
CCCCAAGTCAACATAGTAAAGAAAACTGAGGTGCAGACCGGAGGGTTTTCAAAGGAATCGATTC
TTCCAAAAAGGAATAGTGATAAGCTCATCGCTCGTAAAAAGGACTGGGACCCGAAAAAGTACGG
TGGCTTCGATAGCCCTACAGTTGCCTATTCTGTCCTAGTAGTGGCAAAAGTTGAGAAGGGAAAA
TCCAAGAAACTGAAGTCAGTCAAAGAATTATTGGGGATAACGATTATGGAGCGCTCGTCTTTTG
AAAAGAACCCCATCGACTTCCTTGAGGCGAAAGGTTACAAGGAAGTAAAAAAGGATCTCATAAT
TAAACTACCAAAGTATAGTCTGTTTGAGTTAGAAAATGGCCGAAAACGGATGTTGGCTAGCGCC
GGAGAGCTTCAAAAGGGGAACGAACTCGCACTACCGTCTAAATACGTGAATTTCCTGTATTTAG
CGTCCCATTACGAGAAGTTGAAAGGTTCACCTGAAGATAACGAACAGAAGCAACTTTTTGTTGA
GCAGCACAAACATTATCTCGACGAAATCATAGAGCAAATTTCGGAATTCAGTAAGAGAGTCATC
CTAGCTGATGCCAATCTGGACAAAGTATTAAGCGCATACAACAAGCACAGGGATAAACCCATAC
GTGAGCAGGCGGAAAATATTATCCATTTGTTTACTCTTACCAACCTCGGCGCTCCAGCCGCATT
CAAGTATTTTGACACAACGATAGATCGCAAACGATACACTTCTACCAAGGAGGTGCTAGACGCG
ACACTGATTCACCAATCCATCACGGGATTATATGAAACTCGGATAGATTTGTCACAGCTTGGGG
GTGAG
RNA scaffold expression cassette (S. pyogenes), containing a 20-nucleotide
programmable sequence, a CRISPR RNA motif, and an MS2 operator motif (SEQ ID
No. 23):
N₂₀GTTTTAGAGCTAGAAATAGCAAGTTAAAATAAGGCTAGTCCGTTATCAACTTGAAA
AAGTGGCACCGAGTCGGTGCGCGCACATGAGGATCACCCATGTGCTTTTTTTG
(N₂₀: programmable sequence; Underlined: CRISPR RNA motif; Bold: MS2 motif;
Italic: terminator)

The above RNA scaffold containing one MS2 loop (1×MS2). Shown below is an RNA scaffold containing two MS2 loops (2×MS2), where MS2 scaffolds are underlined:

(SEQ ID No: 24)
GTTTTAGAGCTAGAAATAGCAAGTTAAAATAAGGCTAGTCCGTTATCA
ACTTGAAAAAGTGGCACCGAGTCGGTGCgggagcACATGAGGATCACC
CATGTgccacgagcgACATGAGGATCACCCATGTcgctcgtgttcccT
TTTTTTCTCCGCT
Effector AID -MCP fusion (SEQ ID No. 25):
MDSLLMNRRKFLYQFKNVRWAKGRRETYLCYVVKRRDSATSFSLDFGY
LRNKNGCHVELLFLRYISDWDLDPGRCYRVTWFTSWSPCYDCARHVAD
FLRGNPNLSLRIFTARLYFCEDRKAEPEGLRRLHRAGVQIAIMTFKDY
FYCWNTFVENHERTFKAWEGLHENSVRLSRQLRRILLPLYEVDDLRDA

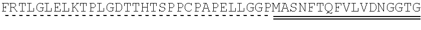

DVTVAPSNFANGIAEWISSNSRSQAYKVTCSVRQSSAQNRKYTIKVEV
PKGAWRSYLNMELTIPIFATNSDCELIVKAMQGLLKDGNPIPSAIAAN
SGIY

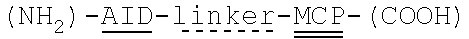

Like the Cas protein described above, the non-nuclease effector can also be obtained as a recombinant polypeptide. Techniques for making recombinant polypeptides are known in the art. See e.g., Creighton, “Proteins: Structures and Molecular Principles,” W.H. Freeman & Co., NY, 1983); Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, N.Y., 2001).

As described herein, by mutating Ser38 to Ala in AID one can reduce the recruitment of AID to off-target sites. Listed below are the DNA and protein sequences of both wild type AID as well as AID_S38A (phosphorylation null, pnAID):

wtAID cDNA (Ser38 codon in bold and underlined, SEQ ID NO: 26):
ATGGACAGCCTCTTGATGAACCGGAGGAAGTTTCTTTACCAATTCAAAAATGTCCGCTGGGCTA
AGGGTCGGCGTGAGACCTACCTGTGCTACGTAGTGAAGAGGCGTGACAGTGCTACATCCTTTTC
ACTGGACTTTGGTTATCTTCGCAATAAGAACGGCTGCCACGTGGAATTGCTCTTCCTCCGCTAC
ATCTCGGACTGGGACCTAGACCCTGGCCGCTGCTACCGCGTCACCTGGTTCACCTCCTGGAGCC
CCTGCTACGACTGTGCCCGACATGTGGCCGACTTTCTGCGAGGGAACCCCAACCTCAGTCTGAG
GATCTTCACCGCGCGCCTCTACTTCTGTGAGGACCGCAAGGCTGAGCCCGAGGGGCTGCGGCGG
CTGCACCGCGCCGGGGTGCAAATAGCCATCATGACCTTCAAAGATTATTTTTACTGCTGGAATA
CTTTTGTAGAAAACCATGAAAGAACTTTCAAAGCCTGGGAAGGGCTGCATGAAAATTCAGTTCG
TCTCTCCAGACAGCTTCGGCGCATCCTTTTGCCCCTGTATGAGGTTGATGACTTACGAGACGCA
TTTCGTACTTTGGGACTT
wtAID protein (Ser38 in bold and underlined, SEQ ID NO: 27):
MDSLLMNRRKFLYQFKNVRWAKGRRETYLCYVVKRRDSATSFSLDFGYLRNKNGCHVELLFLRY
ISDWDLDPGRCYRVTWFTSWSPCYDCARHVADFLRGNPNLSLRIFTARLYFCEDRKAEPEGLRR
LHRAGVQIAIMTFKDYFYCWNTFVENHERTFKAWEGLHENSVRLSRQLRRILLPLYEVDDLRDA
FRTLGL
AID_S38A cDNA (S38A mutation in bold and underlined, SEQ ID NO: 28)
ATGGACAGCCTCTTGATGAACCGGAGGAAGTTTCTTTACCAATTCAAAAATGTCCGCTGGGCTA
AGGGTCGGCGTGAGACCTACCTGTGCTACGTAGTGAAGAGGCGTGACGCCGCTACATCCTTTTC
ACTGGACTTTGGTTATCTTCGCAATAAGAACGGCTGCCACGTGGAATTGCTCTTCCTCCGCTAC
ATCTCGGACTGGGACCTAGACCCTGGCCGCTGCTACCGCGTCACCTGGTTCACCTCCTGGAGCC
CCTGCTACGACTGTGCCCGACATGTGGCCGACTTTCTGCGAGGGAACCCCAACCTCAGTCTGAG
GATCTTCACCGCGCGCCTCTACTTCTGTGAGGACCGCAAGGCTGAGCCCGAGGGGCTGCGGCGG
CTGCACCGCGCCGGGGTGCAAATAGCCATCATGACCTTCAAAGATTATTTTTACTGCTGGAATA
CTTTTGTAGAAAACCATGAAAGAACTTTCAAAGCCTGGGAAGGGCTGCATGAAAATTCAGTTCG
TCTCTCCAGACAGCTTCGGCGCATCCTTTTGCCCCTGTATGAGGTTGATGACTTACGAGACGCA
TTTCGTACTTTGGGACTT
AID_S38A protein (S38A mutation in bold and underlined, SEQ ID NO: 29)
MDSLLMNRRKFLYQFKNVRWAKGRRETYLCYVVKRRDAATSFSLDFGYLRNKNGCHVELLFLRY
ISDWDLDPGRCYRVTWFTSWSPCYDCARHVADFLRGNPNLSLRIFTARLYFCEDRKAEPEGLRR
LHRAGVQIAIMTFKDYFYCWNTFVENHERTFKAWEGLHENSVRLSRQLRRILLPLYEVDDLRDA
FRTLGL

Exemplary Sequences

Shown below are a number of exemplary sequences developed in this study.
1. Protein sequence of RNA scaffold mediated recruitment system (Generation 1 AID) construct (SEQ ID NO: 30):

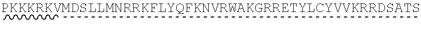

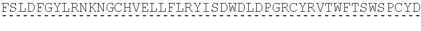

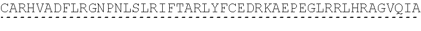

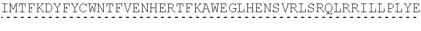

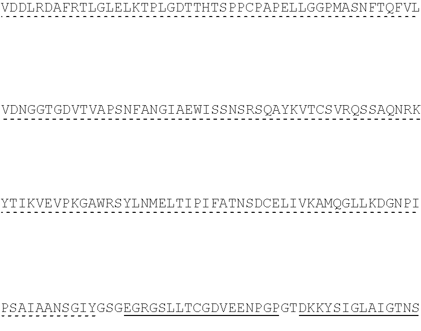

VDNGGTGDVTVAPSNFANGIAEWISSNSRSQAYKVTCSVRQSSAQNRK
YTIKVEVPKGAWRSYLNMELTIPIFATNSDCELIVKAMQGLLKDGNPI
PSAIAANSGIYGSGEGRGSLLTCGDVEENPGPGTDKKYSIGLAIGTNS
VGWAVITDEYKVPSKKFKVLGNTDRHSIKKNLIGALLFDSGETAEATR
LKRTARRRYTRRKNRICYLQEIFSNEMAKVDDSFFHRLEESFLVEEDK
KHERHPIFGNIVDEVAYHEKYPTIYHLRKKLVDSTDKADLRLIYLALA
HMIKFRGHFLIEGDLNPDNSDVDKLFIQLVQTYNQLFEENPINASGVD
AKAILSARLSKSRRLENLIAQLPGEKKNGLFGNLIALSLGLTPNFKSN
FDLAEDAKLQLSKDTYDDDLDNLLAQIGDQYADLFLAAKNLSDAILLS
DILRVNTEITKAPLSASMIKRYDEHHQDLTLLKALVRQQLPEKYKEIF
FDQSKNGYAGYIDGGASQEEFYKFIKPILEKMDGTEELLVKLNREDLL
RKQRTFDNGSIPHQIHLGELHAILRRQEDFYPFLKDNREKIEKILTFR
IPYYVGPLARGNSRFAWMTRKSEETITPWNFEEVVDKGASAQSFIERM
TNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASL
GTYHDLLKIIKDKDFLDNEENEDILEDIVLTLTLFEDREMIEERLKTY
AHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKK
GILQTVKVVDELVKVMGRHKPENIVIEMARENQTTQKGQKNSRERMKR
IEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQI
TKHVAQILDSRMNTKYDENDKLIREVKVITLKSKLVSDFRKDFQFYKV
REINNYHHAHDAYLNAVVGTALIKKYPKLESEFVYGDYKVYDVRKMIA
KSEQEIGKATAKYFFYSNIMNFFKTEITLANGEIRKRPLIETNGETGE
IVWDKGRDFATVRKVLSMPQVNIVKKTEVQTGGFSKESILPKRNSDKL
IARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLA
SAGELQKGNELALPSKYVNFLYLASHYEKLKGSPEDNEQKQLFVEQHK
HYLDEIIEQISEFSKRVILADANLDKVLSAYNKHRDKPIREQAENIIH
LFTLTNLGAPAAFKYFDTTIDRKRYTSTKEVLDATLIHQSITGLYETR
IDLSQLGGDSGGSTNLSDIIEKETGKQLVIQESILMLPEEVEEVIGNK
PESDILVHTAYDESTDENVMLLTSDAPEYKPWALVIQDSNGENKIKML

### Key:

Nuclear Localization Signal (NLS), AID-Linker-MCP, T2A Peptide nCAS9_{D10A}, UGI
2. Protein sequence of RNA scaffold mediated recruitment system(Generation 2 AID) construct (SEQ ID NO: 31):

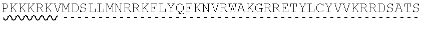

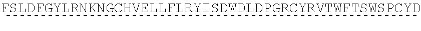

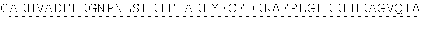

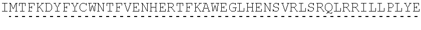

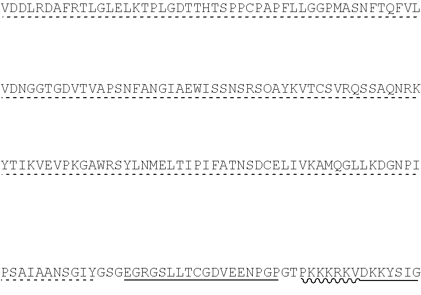

LAIGTNSVGWAVITDEYKVPSKKFKVLGNTDRHSIKKNLIGALLFDSG
ETAEATRLKRTARRRYTRRKNRICYLQEIFSNEMAKVDDSFFHRLEES
FLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENP
INASGVDAKAILSARLSKSRRLENLIAQLPGEKKNGLFGNLIALSLGL
TPNFKSNFDLAEDAKLQLSKDTYDDDLDNLLAQIGDQYADLFLAAKNL
SDAILLSDILRVNTEITKAPLSASMIKRYDEHHQDLTLLKALVRQQLP
EKYKEIFFDQSKNGYAGYIDGGASQEEFYKFIKPILEKMDGTEELLVK
LNREDLLRKQRTFDNGSIPHQIHLGELHAILRRQEDFYPFLKDNREKI
EKILTFRIPYYVGPLARGNSRFAWMTRKSEETITPWNFEEVVDKGASA
QSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVE
DRFNASLGTYHDLLKIIKDKDFLDNEENEDILEDIVLTLTLFEDREMI
EERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLA
GSPAIKKGILQTVKVVDELVKVMGRHKPENMEMARENQTTQKGQKNSR
ERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLV
ETRQITKHVAQILDSRMNTKYDENDKLIREVKVITLKSKLVSDFRKDF
QFYKVREINNYHHAHDAYLNAVVGTALIKKYPKLESEFVYGDYKVYDV
RKMIAKSEQEIGKATAKYFFYSNIMNFFKTEITLANGEIRKRPLIETN
GETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGR
KRMLASAGELQKGNELALPSKYVNFLYLASHYEKLKGSPEDNEQKQLF
VEQHKHYLDEIIEQISEFSKRVILADANLDKVLSAYNKHRDKPIREQA
ENIIHLFTLTNLGAPAAFKYFDTTIDRKRYTSTKEVLDATLIHQSITG
LYETRIDLSQLGGDSGGSGGSGGSTNLSDIIEKETGKQLVIQESILML
PEEVEEVIGNKPESDILVHTAYDESTDENVMLLTSDAPEYKPWALVIQ
DSNGENKIKMLSGGSGGSGGSTNLSDIIEKETGKQLVIQESILMLPEE
EVEEVIGNKPESDILVHTAYDESTDENVMLLTSDAPEYKPWALVIQDS

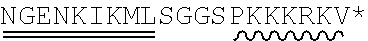

### Key:

NLS, AID-Linker-MCP, T2A Peptide nCAS9_{D10A}, UGI, UGI, NLS

Protein sequence of RNA scaffold mediated recruit-
ment system (Generation 1 APOBEC1) construct (SEQ
ID NO: 32):
PKKKRKVMSSETGPVAVDPTLRRRIEPHEFEVFFDPRELRKETCLLYEIN
WGGRHSIWRHTSQNTNKHVEVNFIEKFTTERYFCPNTRCSITWFLSWSPC
GECSRAITEFLSRYPHVTLFIYIARLYHHADPRNRQGLRDLISSGVTIQI
MTEQESGYCWRNFVNYSPSNEAHWPRYPHLWVRLYVLELYCIILGLPPCL
NILRRKQPQLTFFTIALQSCHYQRLPPHILWATGLKELKTPLGDTTHTSP
PCPAPELLGGPMASNFTQFVLVDNGGTGDVTVAPSNFANGIAEWISSNSR
SQAYKVTCSVRQSSAQNRKYTIKVEVPKGAWRSYLNMELTIPIFATNSDC
ELIVKAMQGLLKDGNPIPSAIAANSGIYGSGEGRGSLLTCGDVEENPGPG
TDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDSGGSTNLSDIIEKETGKQLVIQESILMLPEEV
EEVIGNKPESDILVHTAYDESTDENVMLLTSDAPEYKPWALVIQDSNGEN
KIKMLSGGSPKKKRKV*

### Key:

NLS, APOBEC1—linker-MCP, T2A peptide nCAS9D10A, UGI, NLS

Protein sequence of RNA scaffold mediated
recruitment system (Generation 2 APOBEC1)
construct (SEQ ID NO: 33):

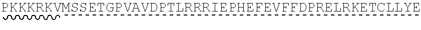

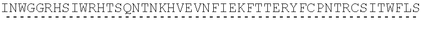

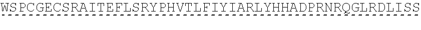

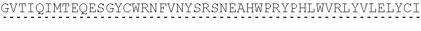

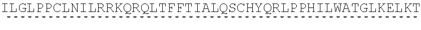

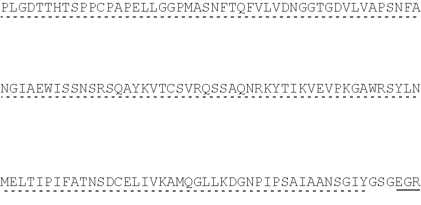

GSLLTCGDVEENPGPGTDKKYSIGLAIGTNSVGWAVITDEYKVPSKKF
KVLGNTDRHSIKKNLIGALLFDSGETAEATRLKRTARRRYTRRKNRIC
YLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNP
DNSDVDKLFIQLVQTYNQLFEENPINASGVDAKAILSARLSKSRRLEN
LIAQLPGEKKNGLFGNLIALSLGLTPNFKSNFDLAEDAKLQLSKDTYD
DDLDNLLAQIGDQYADLFLAAKNLSDAILLSDILRVNTEITKAPLSAS
MIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHL
GELHAILRRQEDFYPFLKDNREKIEKILTFRIPYYVGPLARGNSRFAW
MTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLD
NEENEDILEDIVLTLTLFEDREMIEERLKTYAHLFDDKVMKQLKRRRY
TGWGRLSRKLINGIRDKQSGKTILDFLKSDGFANRNFMQLIHDDSLTF
KEDIQKAQVSGQGDSLHEHIANLAGSPAIKKGILQTVKVVDELVKVMG
RHKPENIVIEMARENQTTQKGQKNSRERMKRIEEGIKELGSQILKEHP
VENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRK
FDNLTKAERGGLSELDKAGFIKRQLVETRQITKHVAQILDSRMNTKYD
ENDKLIREVKVITLKSKLVSDFRKDFQFYKVREINNYHHAHDAYLNAV
VGTALIKKYPKLESEFVYGDYKVYDVRKMIAKSEQEIGKATAKYFFYS
NIMNFFKTEITLANGEIRKRPLIETNGETGEIVWDKGRDFATVRKVLS
MPQVNIVKKTEVQTGGFSKESILPKRNSDKLIARKKDWDPKKYGGFDS
PTVAYSVLVVAKVEKGKSKKLKSVKELLGITIMERSSFEKNPIDFLEA
KGYKEVKKDLIIKLPKYSLFELENGRKRMLASAGELQKGNELALPSKY
VNFLYLASHYEKLKGSPEDNEQKQLFVEQHKHYLDEIIEQISEFSKRV
ILADANLDKVLSAYNKHRDKPIREQAENIIHLFTLTNLGAPAAFKYFD
TTIDRKRYTSTKEVLDATLIHQSITGLYETRIDLSQLGGDSGGSTNLS
DIIEKETGKQLVIQESILMLPEEVEEVIGNKPESDILVHTAYDESTDE

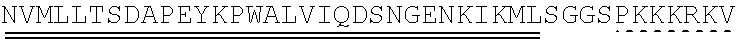

### Key:

NLS, APOBEC1—linker-MCP, T2A peptide nCAS9D10A, UGI

Shown below are a number of exemplary RNA sequence of gRNA constructs used in this study. Each contains, from the 3′ end to the 5′ end, a customizable target, a gRNA scaffold, and one or two copies of a MS2 aptamer.

1. Sequence of gRNA_MS2 construct (SEQ. ID NO: 34):

NNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGCg

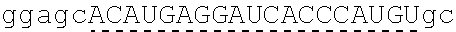

2. Sequence of gRNA_2×MS2 construct (SEQ ID NO: 35):

NNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGCg

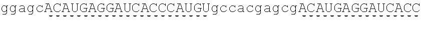

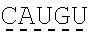

Key: Customizable target-gRNA scaffold-MS2 aptamers

The above three components of the platform/system disclosed herein can be expressed using one, two or three expression vectors. The system can be programmed to target virtually any DNA or RNA sequence. In addition to the second generation RNA scaffold mediated base editors described above, similar second generation RNA scaffold mediated base editors could be generated by varying the modular components of the system, including any suitable Cas orthologs, deaminase orthologs, and other DNA modification enzymes.

Cell Types/Therapeutic Uses

Immune cells, particularly primary immune cells, either naturally occurring within a host animal or patient, or immune cells derived from an induced pluripotent stem cell (iPSC) may be genetically modified using the methods and system provided herein. Immune cells include T cells, Natural Killer (NK) cells, B cells, myeloblasts, erythroblasts and pluripotent cells that are immune cell precursors, such as haematopoietic stem cells (HSCs) which can differentiate into all blood and immune cells. Haemopoietic stem cells (HSCs) arise from hemangioblasts, which can give rise to HSCs, vascular smooth muscle cells and angioblasts, which differentiate into vascular endothelial cells. HSCs can give rise to common myeloid and common lymphoid progenitors from which arise T cells, Natural Killer (NK) cells, B cells, myeloblasts, erythroblasts and other cells involved in the production of cells of blood, bone marrow, spleen, lymph nodes, and thymus. Induced pluripotent stem cell [iPSC] may be genetically modified using the methods and system provided herein and then may be subsequently differentiated to produce a desired cell type, for example, an immune cell, such as a T-cell.

Provided herein are also methods for genome engineering (e.g., for altering or manipulating the expression of one or more genes or one or more gene products) in prokaryotic or eukaryotic cells, in vitro, in vivo, or ex vivo. In particular, the methods provided herein are useful for targeted base editing disruption in mammalian cells including primary human T cells, induced pluripotent stem cells (iPSCs) natural killer (NK) cells, CD34+ hematopoietic stem and progenitor cells (HSPCs), such as HSPCs isolated from umbilical cord blood or bone marrow and cells differentiated from them.

Also provided herein are genetically engineered cells arising from haematopoietic stem cells, such as T cells, that have been modified according to the methods described herein.

In some cases, the methods are configured to produce genetically engineered T cells arising from HSCs or iPSCs, that are suitable as “universally acceptable” cells for therapeutic application. Such methods can also be applied to natural killer (NK) cells, CD34+ hematopoietic stem and progenitor cells (HSPCs), such as HSPCs isolated from umbilical cord blood or bone marrow and cells differentiated from them.

Sequences of exemplary gRNAs for editing target bases in genetically engineered T cells include TRAC, TRBC1, TRBC2, PDCD1, CD52 and B2M are set forth in Table 5.

Adoptive immunotherapy, which involves the transfer of autologous antigen-specific T-cells generated ex vivo, is a promising strategy to treat cancer. The T cells used for adoptive immunotherapy can be generated either by expansion of antigen-specific T cells or redirection of T cells through genetic engineering. Transfer of viral antigen specific T-cells is a well-established procedure used for the treatment of transplant associated viral infections and rare viral-related malignancies. Similarly, isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. CARs have successfully allowed T cells to be redirected against antigens expressed at the surface of tumor cells from various malignancies including lymphomas and solid tumors.

As will be apparent to the skilled person, the methods provided herein may be used to genetically modify an immune cell that have previously been genetically modified e.g. to express a chimeric antigen receptor (CAR) and/or T cell receptor (TCR). For example, the base editing methods disclosed herein may be used to modify a T cell that has already been engineered to express a CAR or TCR. For example, the methods provided herein may be used to functionally knock-out one or more genes in a CAR or TCR expressing T cell.

In another aspect, provided herein are methods for targeting diseases for base editing correction. The target sequence can be any disease-associated polynucleotide or gene, as have been established in the art. Examples of useful applications of mutation or ‘correction’ of an endogenous gene sequence include alterations of disease-associated gene mutations, alterations in sequences encoding splice sites, alterations in regulatory sequences, alterations in sequences to cause a gain-of-function mutation, and/or alterations in sequences to cause a loss-of-function mutation, and targeted alterations of sequences encoding structural characteristics of a protein.

In some cases, it will be advantageous to genetically modify a cell using the methods described herein such that cell expresses a chimeric antigen receptor (CAR) and/or T cell receptor (TCR). The “chimeric antigen receptor” (CAR)” is sometimes called a “chimeric receptor”, a “T-body”, or a “chimeric immune receptor” (CIR). As used herein, the term “chimeric antigen receptor” (CAR) refers to an artificially constructed hybrid protein or polypeptide comprising an extracellular antigen binding domains of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, T cells can be engineered to express CAR specific for CD19 on B-cell lymphoma. For allogenic antitumor cell therapeutics not limited by donor-matching, cells can be engineered to knock-in nucleic acids encoding a CAR but also knocking out genes responsible for donor matching (TCR,HLA markers, and genes related to MHC I and MHC II).

First generation CAR constructs comprise a binding domain (a scFv antibody), a hinge region, a transmembrane domain and an intracellular signalling domain (Liu et al, 2019, Frontiers in Immunology). Yescarta™ (Axicabtagene ciloleucel) was approved for use in 2017 for the treatment of large B-cell lymphoma that has failed conventional treatment and was one of the first therapies of this type. It employs a binding domain that targets CD19, a protein expressed by normal B cells, B cell leukemias and lymphomas. The second generation CAR (Kochenderfer et al 2009, J Immunotherapy) used in this therapy consists of an anti-CD19 scFv derived from the FMC63 mouse hybridoma (Nicholson et al 1997, Mol Immunology), a portion of the human CD28 molecule (a hinge extracellular part, a transmembrane domain and the entire intracellular domain) and the entire domain of CD3-zeta chain.

In one embodiment the binding domain is a scFv.

In a particular embodiment the binding domain is an anti-CD19 scFv.

In another embodiment the binding domain is an anti-B-cell maturation antigen (BCMA) scFv.

The intracellular signalling domain effects signalling inside the cell via phosphorylation of CD3-zeta following antigen binding. CD3-zeta's cytoplasmic domain is routinely used as the main CAR endodomain component. Other co-stimulatory molecules in addition to CD3 signalling are also required for T cell activation and so CAR receptors typically include co-stimulatory molecules including CD28, CD27, CD134 (Ox40) and CD137 (4-1BB). Examples of first, second, third and fourth generation CAR are described in Subklewe M et al, Transfusion Medicine and Hemotherapy. 2019 February; 46(1):15-24.

In a particular embodiment the intracellular signalling domain is the entire intracellular domain of CD3-zeta chain.

In one embodiment the intracellular signalling additionally comprises 41BB-CD3-zeta chain or CD28-CD3-zeta chain.

The hinge region is typically a small structural spacer that sits between the binding domain and the cells outer membrane. Ideally it enhances the flexibility of the scFv to reduce special constraints between the CAR and its target antigen. Design of the hinge region has been described in the art and typically is based on sequences which are membrane proximal regions from other immune molecules such as IgG, CD8 and CD28 (Chandran, S S et al 2019, Immunological Reviews, 290 (1):127-147 and Qin L, et al 2017, Journal of Hematological Oncology. 10 (1) 68).

The transmembrane domain is a structural component consisting of a hydrophobic alpha helix that spans the cell membrane. It functions by anchoring the CAR to the plasma membrane thereby bridging the hinge region and binding domain with the intracellular signalling domain. The CD28 transmembrane domain is typically used in CARs and is known to result in a stably expressed receptor.

As used herein, the terms “genetically modified” and “genetically engineered” are used interchangeably and refer to a prokaryotic or eukaryotic cell that includes an exogenous polynucleotide, regardless of the method used for insertion. In some cases, the effector cell has been modified to comprise a non-naturally occurring nucleic acid molecule that has been created or modified by the hand of man (e.g., using recombinant DNA technology) or is derived from such a molecule (e.g., by transcription, translation, etc.). An effector cell that contains an exogenous, recombinant, synthetic, and/or otherwise modified polynucleotide is considered to be an engineered cell.

Cell Therapies and Ex Vivo Therapies

Various embodiments of the present invention also provide cells that are produced or used in accordance with any of the other embodiments of the present invention for use in therapy. In one embodiment, the present invention is directed to methods for generating therapeutic cells such as T cells engineered to express a Chimeric Antigen Receptor (CAR-T) or T Cell Receptor (TCR-T). The CAR-T/TCR-T cells may be derived from primary T cells or differentiated from stem cells. Suitable stem cells include, but are not limited to, mammalian stem cells such as human stem cells, including, but not limited to, hematopoietic, neural, embryonic, induced pluripotent stem cells (iPSC), mesenchymal, mesodermal, liver, pancreatic, muscle, and retinal stem cells. Other stems cells include, but are not limited to, mammalian stem cells such as mouse stem cells, e.g., mouse embryonic stem cells.

In various embodiments, the present invention may be used to knockout, modify or increase the expression of a single gene or multiple genes in various types of cells or cell lines, including but not limited to cells from mammals. The present systems and methods may be applicable to multiplex genetic modification, which, as in known in the art, involves genetically modifying multiple genes or multiple targets within the same gene. The technology may be used for many applications, including but not limited to knock out of genes to prevent graft versus host disease by making non-host cells non-immunogenic to the host or prevent host vs graft disease by making non-host cells resistant to attack by the host. These approaches are also relevant to generating allogenic (off-the-shelf) or autologous (patient specific) cell-based therapeutics. Such genes include, but are not limited to, the T Cell Receptor (TRAC, TRBC1. TRBC2, TRDC, TRGC1, TRGC2), the major histocompatibility complex (MHC class I and class II) genes, including B2M, co-receptors (HLA-F, HLA-G), genes involved in the innate immune response (MICA, MICB, HCP5, STING, DDX41 and Toll-like-receptors (TLRs)), inflammation (NKBBiL, LTA, TNF, LTB, LST1, NCR3, AIF1), heat shock proteins (HSPA1L, HSPA1A, HSPA1B), complement cascade, regulatory receptors (NOTCH family members), antigen processing (TAP, HLA-DM, HLA-DO), increased potency or persistence (such as PD-1, CTLA-4 and other members of the B7 family of checkpoint proteins), genes involved in immunosuppressive immune cells (such as FOXP3 and Interleukin (IL)-10), genes involved in T cell interaction with the tumour microenvironment (including but not limited to receptors of cytokines such as TGFB, IL-4, IL-7, IL-2, IL-15, IL-12, IL-18, IFNgamma), genes involved in contributing to cytokine release syndrome (including but not limited to IL-6, IFNgamma, IL-8 (CXCL8), IL-10, GM-CSF, MIP-1α/β, MCP-1 (CCL2), CXCL9, and CXCL10 (IP-10), genes that code for the antigen targeted by a CAR/TCR (for example endogenous CS1 where the CAR is designed against CS1) or other genes found to be beneficial to CAR-T/TCR-T (such as TET2, ARG2, NR4A1, NR4A2, NR4A3, TOX and TOX2) or other cell based therapeutics including but not limited to CAR-NK, CAR-B etc. See, e.g., DeRenzo et al., Genetic Modification Strategies to Enhance CAR T Cell Persistence for Patients With Solid Tumors. Front. Immunol., 15 Feb. 2019.

The technology may also be used to knock down or modify genes that are involved in fratricide of immune cells, such as T cells and NK cells, or genes that alert the immune system of a patient or animal that a foreign cell, particle or molecule has entered a patient or animal, or genes encoding proteins that are current therapeutic targets used to compromise or boost an immune response, for example, CD52 and PD1, respectively.

One application of the method and system provided herein is to engineer HLA alleles of bone marrow cells or bone marrow cells differentiated from iPS cells to increase haplotype match. The engineered cells can be used for bone marrow transplantation for treating leukemia. Another application is to engineer the negative regulatory element of fetal hemoglobin gene in hematopoietic stem cells for treating sickle cell anemia and beta-thalassemia. The negative regulatory element will be mutated and the expression of fetal hemoglobin gene is re-activated in hematopoietic stem cells, compensating the functional loss due to mutations in adult alpha or beta hemoglobin genes. A further application is to engineer iPS cells for generating allogenic therapeutic cells for various degenerative diseases including Parkinson's disease (neuronal cell loss), Type 1 diabetes (pancreatic beta cell loss). Other exemplary applications include engineering HIV infection resistant T-Cells by inactivating CCR5 gene and other genes encoding receptors required for HIV entering cells; removing a premature stop codon in the DMD gene to re-establish expression of dystrophin; and the correction of cancer driver mutations, such as p53 Y163C.

Type of Genetic Modifications

Accordingly, provided herein are methods for targeted disruption of transcription or translation of a target gene. In particular, the methods comprise targeted disruption of transcription or translation of a target gene via disruption of a start codon, introduction of a premature stop codon, and/or targeted disruption of intron/exon splice sites.

Using the methods described herein, one may knock-in and/or knock-out one or more genes of interest in primary cells with improved efficiency and a reduced rate of off-target indel formation. In preferred embodiments, the methods are used for multiplexed base editing comprising gene knock-in, gene knock-out, and missense mutation.

As described in the paragraphs and Examples that follow, the inventors' streamlined approach to genome engineering employs base editors for applications including targeted gene disruption by knock-out and missense mutation and gene correction. The methods described herein are well-suited for studying immune cell biology and gene function, modeling diseases such as primary immunodeficiencies, as well as correcting disease-causing point mutations, and generating novel cell products (e.g., T cell products) for therapeutic applications.

Disruptions at splice acceptor-splice donor (SA-SD) sites are also useful because one may knock out coding sequence and non-coding RNAs (ncRNAs) without stop codon read through.

Exemplary SA-SD gRNAs designed toward human long non-coding RNAs and human protein coding genes relevant to immunotherapy are set forth in Table 5.

Delivery of Components into Cells

Suitable methods for delivering the base editing components to immune cells such as haemopoietic cells are provided in the examples herein below.

In embodiments provided herein the guide RNA molecule can be delivered to the target cell via various methods, without limitation, listed below. Firstly, direct introduction of synthetic RNA molecules (whether sgRNA, crRNA, or tracrRNA and modifications thereof) to the cell of interest by electroporation, nucleofection, transfection, via nanoparticles, via viral mediated RNA delivery, via non-viral mediated delivery, via extracellular vesicles (for example exosome and microvesicles), via eukaryotic cell transfer (for example by recombinant yeast) and other methods that can package the RNA, molecules and can be delivered to the target viable cell without changes to the genomic landscape. Other methods for the introduction of guide RNA molecules include non-integrative transient transfer of DNA polynucleotides that includes the relevant sequence for the protein recruitment so that the molecule can be transcribed into the target guide RNA molecule, this includes, without limitation. DNA-only vehicles (for example, plasmids, MiniVectors, MiniStrings, Protelomerase generated DNA molecules (for example Doggybones), artificial chromosome (for example HAC), cosmids), via DNA vehicles by nanoparticles, extracellular vesicles (for example exosome and microvesicles), via eukaryotic cell transfer (for example by recombinant yeast), transient viral transfer by AAV, non-integrating, viral particles (for example lentivirus and retrovirus based systems), cell penetrating peptides and other technology that can mediate the introduction of DNA into a cell without direct integration into the genomic landscape. Another method for the introduction of the guide RNA include the use of integrative gene transfer technology for stable introduction of the machinery for guide RNA transcription into the genome of the target cells, this can be controls via constitutive or promoter inducible systems to attenuate the guide RNA expression and this can also be designed so that the system can be removed after the utility has been met (for example, introducing a Cre-Lox recombination system), such technology for stable gene transfer includes, but not limited to, integrating viral particles (for example lentivirus, adenovirus and retrovirus based systems), transposase mediate transfer (for example Sleeping Beauty and Piggybac), exploitation of the non-homologous repair pathways introduced by DNA breaks (for example utilising CRISPR and TALEN) technology and a surrogate DNA molecule, and other technology that encourages integration of the target DNA into a cell of interest.

The method for delivering deaminase effector fusion protein and the CRISPR sequencing targeting components are often mediated by the same technology but in some situations, there are advantages to mediate the delivery via different methods. The applicable methods, and not limited to, are listed below. Firstly, the direct introduction of mRNA and Protein molecules directly to the cell of interest by electroporation, nucleofection, transfection, via nanoparticles, via viral mediated packaged delivery, extracellular vesicles (for example exosome and microvesicles), via eukaryotic cell transfer (for example by recombinant yeast), and other methods that can package the macromolecules and can be delivered to the target viable cell without integration into genomic landscape. Other methods for the introduction of molecules include non-integrative transient transfer of DNA polynucleotides that includes the relevant sequence for the protein recruitment so that the molecule or molecules can be transcribed and translated into the target protein molecule, this includes, without limitation, DNA-only vehicles (for example, plasmids, MiniCircles, MiniVectors, MiniStrings, Protelomerase generated DNA molecules (for example Doggybones), artificial chromosome (for example HAC), cosmids), via DNA vehicles by nanoparticles, extracellular vesicles (for example exosome and microvesicles), via eukaryotic cell transfer (for example by recombinant yeast), transient viral transfer by AAV, non-integrating viral particles (for example lentivirus and retrovirus based systems), and other technology that can mediate the introduction of DNA into a cell without direct integration into the genomic landscape. Another method for the introduction of the deaminase effector fusion protein and the CRISPR sequencing targeting components include the use of integrative gene transfer technology for stable introduction of the machinery for transcription and translation into the genome of the target cells, this can be controlled via constitutive or inducible promoter systems to attenuate the molecule, or molecules expression, and this can also be designed so that the system can be removed after the utility has been met (for example, introducing a Cre-Lox recombination system), such technology for stable gene transfer includes, but not limited to, integrating viral particles (for example lentivirus, adenovirus and retrovirus based systems), transposase mediate transfer (for example Sleeping Beauty and Piggybac), exploitation of the non-homologous repair pathway's introduced by DNA breaks (for example utilising CRISPR and TALEN) technology and a surrogate DNA molecule, and other technology that encourages integration of the target DNA into a cell of interest.

Expression System

To use the platform described above, it may be desirable to express one or more of the protein and RNA components from nucleic acids that encode them. This can be performed in a variety of ways. For example, the nucleic acids encoding the RNA scaffold or proteins can be cloned into one or more intermediate vectors for introducing into prokaryotic or eukaryotic cells for replication and/or transcription. Intermediate vectors are typically prokaryotic vectors, e.g., plasmids, or shuttle vectors, or insect vectors, for storage or manipulation of the nucleic acid encoding the RNA scaffold or protein for production of the RNA scaffold or protein. The nucleic acids can also be cloned into one or more expression vectors, for administration to a plant cell, animal cell, preferably a mammalian cell or a human cell, fungal cell, bacterial cell, or protozoan cell. Accordingly, the present invention provides nucleic acids that encode any of the RNA scaffold or proteins mentioned above. Preferably, the nucleic acids are isolated and/or purified.

The present invention also provides recombinant constructs or vectors having sequences encoding one or more of the RNA scaffold or proteins described above. Examples of the constructs include a vector, such as a plasmid or viral vector, into which a nucleic acid sequence of the invention has been inserted, in a forward or reverse orientation. In a preferred embodiment, the construct further includes regulatory sequences, including a promoter, operably linked to the sequence. Large numbers of suitable vectors and promoters are known to those of skill in the art and are commercially available. Appropriate cloning and expression vectors for use with prokaryotic and eukaryotic hosts are also described in e.g., Sambrook et al. (2001, Molecular Cloning: A Laboratory Manual, Cold Spring Harbor Press).

A vector refers to a nucleic acid molecule capable of transporting another nucleic acid to which it has been linked. The vector can be capable of autonomous replication or integration into a host DNA. Examples of the vector include a plasmid, cosmid, or viral vector. The vector of this invention includes a nucleic acid in a form suitable for expression of the nucleic acid in a host cell. Preferably, the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the host cell to be transformed, transfected, or transduced, the level of expression of RNAs or proteins desired, and the like.

Examples of expression vectors include chromosomal, non-chromosomal and synthetic DNA sequences, bacterial plasmids, phage DNA, baculovirus, yeast plasmids, vectors derived from combinations of plasmids and phage DNA, viral DNA such as vaccinia, adenovirus, fowl pox virus, and pseudorabies. However, any other vector may be used provided it is replicable and viable in the host. The appropriate nucleic acid sequence may be inserted into the vector by a variety of procedures. In general, a nucleic acid sequence encoding one of the RNAs or proteins described above can be inserted into an appropriate restriction endonuclease site(s) by procedures known in the art. Such procedures and related sub-cloning procedures are within the scope of those skilled in the art.

The vector may include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli.

The vectors for expressing the guide RNAs can include RNA Pol III promoters to drive expression of the RNAs, e.g., the HI, U6 or 7SK promoters. These human promoters allow for expression of RNAs in mammalian cells following transfection. Alternatively, a T7 promoter may be used, e.g., for in vitro transcription, and the RNA can be transcribed in vitro and purified.

The vector containing the appropriate nucleic acid sequences as described above, as well as an appropriate promoter or control sequence, can be employed to transform, transfect, or infect an appropriate host to permit the host to express the RNAs or proteins described above. Examples of suitable expression hosts include bacterial cells (e.g., E. coli, Streptomyces, Salmonella typhimurium), fungal cells (yeast), insect cells (e.g., Drosophila and Spodoptera frugiperda (Sf9), animal cells (e.g., CHO, COS, and HEK 293), adenoviruses, and plant cells. The selection of an appropriate host is within the scope of those skilled in the art. In some embodiments, the present invention provides methods for producing the above mentioned RNAs or proteins by transforming, transfecting, or infecting a host cell with an expression vector having a nucleotide sequence that encodes one of the RNAs, or polypeptides, or proteins. The host cells are then cultured under a suitable condition, which allows for the expression of the RNAs or proteins.

Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

### Culturing the Cells

The method further comprises maintaining the cell under appropriate conditions such that the guide RNA guides the effector protein to the targeted site in the target sequence, and the effector domain modifies the target sequence.

In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Cells useful for the methods provided herein can be freshly isolated primary cells or obtained from a frozen aliquot of a primary cell culture. In some cases, cells are electroporated for uptake of gRNAs and the base editing fusion protein. As described in the Examples that follow, electroporation conditions for some assays (e.g., for T cells) can comprise 1600 volts, pulse width of 10 milliseconds, 3 pulses. Following electroporation, electroporated T cells are allowed to recover in a cell culture medium and then cultured in a T cell expansion medium. In some cases, electroporated cells are allowed to recover in the cell culture medium for about 5 to about 30 minutes (e.g., about 5, 10, 15, 20, 25, 30 minutes). Preferably, the recovery cell culture medium is free of an antibiotic or other selection agent. In some cases, the T cell expansion medium is complete Immunocult-XT Expansion medium.

### Definition

A nucleic acid or polynucleotide refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “isolated” when referring to nucleic acid molecules or polypeptides means that the nucleic acid molecule or the polypeptide is substantially free from at least one other component with which it is associated or found together in nature.

As used herein, the term “guide RNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating CRISPR RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas9 polynucleotides are derived from the wild type Cas9 protein amino acid sequence. Also, the variant mammalian codon-optimized Cas9 polynucleotides, including the Cas9 single mutant nickase (nCas9, such as nCas9D10A) and Cas9 double mutant null-nuclease (dCas9, such as dCas9 D10A H840A), are derived from the polynucleotide encoding the wild type mammalian codon-optimized Cas9 protein.

As used herein the term “wild type” is a term of the art understood by skilled persons and means the typical form of an organism, strain, gene or characteristic as it occurs in nature as distinguished from mutant or variant forms.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. For example, the mutant forms of mammalian codon-optimized Cas9 (hspCas9), including the Cas9 single mutant nickase and the Cas9 double mutant null-nuclease, are variants of the mammalian codon-optimized wild type Cas9 (hspCas9). The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 96%, 97%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule, for example, mutant variants of the Cas9 polypeptide that have modified or lost nuclease activity. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where a substitution within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline Amino acids having polar, uncharged side chains include: serine, threonine, cysteine, methionine, asparagine and glutamine Amino acids having aromatic side chains include: phenylalanine, tyrosine and tryptophan. Amino acids having positively charged side chains include: lysine, arginine and histidine. Amino acids having negatively charged side chains include: aspartate and glutamate.

A “Cas9 mutant” or “Cas9 variant” refers to a protein or polypeptide derivative of the wild type Cas9 protein such as S. pyogenes Cas9 protein (i.e., SEQ ID NO: 20), e.g., a protein having one or more point mutations, insertions, deletions, truncations, a fusion protein, or a combination thereof. It retains substantially the RNA targeting activity of the Cas9 protein. The protein or polypeptide can comprise, consist of, or consist essentially of a fragment of SEQ ID NO: 20. In general, the mutant/variant is at least 50% (e.g., any number between 50% and 100%, inclusive) identical to SEQ ID NO: 20. The mutant/variant can bind to an RNA molecule and be targeted to a specific DNA sequence via the RNA molecule, and may additional have a nuclease activity. Examples of these domains include RuvC like motifs (aa. 7-22, 759-766 and 982-989 in SEQ ID NO: 20) and HNH motif (aa 837-863). See Gasiunas et al., Proc Natl Acad Sci USA. 2012 Sep. 25; 109(39): E2579-E2586 and WO2013176772.

“Complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part I, Second Chapter “Overview of principles of hybridization and the strategy of nucleic acid probe assay”, Elsevier, N.Y.

“Hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytidine and guanine (C and G), other base pairs may form (e.g., Adams et al., The Biochemistry of the Nucleic Acids, 11th ed., 1992).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The terms “subject” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. In some embodiments, a subject may be an invertebrate animal, for example, an insect or a nematode; while in others, a subject may be a plant or a fungus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an Cas protein or guide RNA, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

Various exemplary embodiments of compositions and methods according to this invention are now described in the following Examples.

### EXAMPLES

### Example 1: Base Editing System Applied to Human Primary Immune Cells Utilising Lentiviral Integrated sgRNA

In this example, primary human Pan T lymphocytes were used to prove the utility of the base editing mRNA components in primary immune cells in the presence of a constitutive expression sgRNA with RNA aptamers under the control of a PolIII promoter. The Pan T cells were activated utilising anti-CD3 and anti-CD28 and then transduced using enriched and concentrated lentiviral particles. Successfully transduced cells were selected using puromycin selection to ensure >95% of the population had at least one copy of the lentiviral insert. During the selection the T cells were re-activated by anti-CD3 and anti-CD28, then the cells were electroporated with mRNA components for both the deaminase-MCP and the nCas9-UGI-UGI components. The cells were then incubated for a further 72-96 hours and cells were checked for surface KO by flow cytometry and the base editing was checked by targeted PCR amplification and Sanger sequencing.

The data prove that the base editing system can edit primary immune cells with surprising >95% efficiency by functional protein knock-out on the surface of the cells (FIG. 2), and showing consistent functional Knock-out across sites with good correlation to genomic DNA base changes (FIG. 3+4)

### Example 2: Base Editing System Applied to Human Primary Immune Cells Utilising Synthetic Two-Part crRNA and tracrRNA-Aptamer Guides

In this example, primary human Pan T lymphocytes were used to prove the utility of the base editing system with two-part crRNA and aptamer modified tracrRNA components in primary immune cells. The Pan T cells were activated utilising anti-CD3 and anti-CD28 and then cells were electroporated with mRNA components for both the deaminase-MCP, nCas9-UGI-UGI components, tracrRNA-Aptamer and the crRNA. The cells were then incubated for a further 72-96 hours and cells were checked for surface KO by flow cytometry and the base editing was checked by targeted PCR amplification and Sanger sequencing.

The data show the base editing system can edit primary immune cells, without the necessity to integrate DNA into the genome (via lentiviral cassettes), utilising varied crRNA and tracrRNA-Aptamer with mRNA components. The results display a distinct RNA aptamer and deaminase specificity with the Apobec1 having preferences for the single RNA motif, whilst the AID deaminase preferring the double RNA motif in this context. The results display a high utility of the base editing system for altering specific bases for function protein knock-out by surface staining and flow cytometry and by alterations at the DNA level (FIG. 5+6)

Material and Methods

Guides: Internally generated data was used to specify base editing windows calculated at set distances from the PAM motif (NGG). The data was used to development algorithms to predict Phenotype or Gene KO applicable guides sequence for the following genes: TRAC, TRBC1, TRBC2, PDCD-1, B2M, and CD52 (Table 4). The crRNAs and tracrRNA were synthesised by Horizon Discovery (formerly Dharmacon) and Agilent Technologies.

Synthetic one-part crRNA Sequence (SEQ ID NO: 36):
mN*mN*NNNNNNNNNNNNNNNNNNGUUUUAGAGCUAUGCUGUUUUG
2′OMe (m) and phosphorothioate (*) modified
residues
Synthetic two-part 1xMS2 tracrRNA-Aptamer Sequence
(SEQ ID NO: 37):
AACAGCAUAGCAAGUUAAAAUAAGGCUAGUCCGUUAUCAACUUGAAAAAG
UGGCACCGAGUCGGUGCGCGCACAUGAGGAUCACCCAUGUGCUUUUmU*
mU*U
2′OMe (m) and phosphorothioate (*) modified
residues
Synthetic two-part 2xMS2 tracrRNA-Aptamer Sequence
(SEQ ID NO: 38):
AACAGCAUAGCAAGUUAAAAUAAGGCUAGUCCGUUAUCAACUUGAAAAAG
UGGCACCGAGUCGGUGCGGGAGCACAUGAGGAUCACCCAUGUGCCACGAG
CGACAUGAGGAUCACCCAUGUCGCUCGUGUUCCCUUUUmU*mU*U
2′OMe (m) and phosphorothioate (*) modified
residues
Lentiviral sgRNA sequences (SEQ ID NO: 39):
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGCggga
gcACAUGAGGAUCACCCAUGUgccacgagcgACAUGAGGAUCACCCAUGU
cgcUcgUgUUcccUUUUUUU

mRNA Component Generation: Messenger RNA molecules were custom generated by Trilink utilising modified nucleotides: Pseudouridine and 5-Methyl-Cytosine. The mRNA components translated to the following proteins: Deaminase AID=NLS-hAID-Linker-MCP, Deaminase Apobec1=NLS-rApobec1-Linker-MCP, and Cas9-UGI-UGI=NLS-nCas9-UGI-UGI-NLS

Plasmid Construction: The lentiviral construct included additional selectable markers (e.g. antibiotics, fluorescent proteins) to ensure that single integration copies were present within the genome of the target cell population. Sequences for the specific guides sequences were cloned (by T4 DNA ligase technology) into overhangs generated by Type IIS restriction enzyme sites. The target construct ensured the guide sequence was perfectly in frame for efficient transcription from the human U6 PolIII promoter (inclusion of a G nucleotide if not at the 5′ of the sequence) and to extended into the Cas9 scaffold and aptamer sequences before termination sequences. Plasmid clones were check by Sanger sequencing and restriction digestion QC, before being expanded for large-scale plasmid preparation (e.g. maxiprep).

Lentiviral Particle Generation: sgRNA-Aptamer lentiviral constructs were made in functional lentiviral particles using 3^{rd} generation plasmid systems (Horizon Discovery). Viral particles were then concentrated by diafiltration and aliquoted for transduction.

Lentiviral Transduction: T cells were activated for >48 hours and transduced with a MOI of 0.1 by Retronectin (T100B, Takara-bio) treated plates and incubation at 37 C and 5% CO2 overnight. Frozen T Cells Culturing: Sources of frozen CD3+ T Cells (Hemacare) were thawed and then cultured into Immunocult XT media (STEMCELL Technologies) with 1× Penicillin/Streptomycin (Thermofisher) at 37 C and 5% CO2.

T Cell Electroporation: After 48-72 post-activation T cells were electroporated with using the Neon Electroporator (Thermofisher). Neon Electroporator conditions were 1600v/10 ms/3 pulses with a 10 ul tip with 250 k cells, combined total of mRNA amount of 1-5 ug, for both the Deaminase-MCP and nCas9-UGI-UGI, and where applicable 0.2-1.8 umol of complexed crRNA:tracrRNA. Post-electroporation cells were transferred to Immunocult XT media with 100 U IL-2, 100 U IL-7 and 100 U IL-15 (STEMCELL Technologies) and cultured at 37 C and 5% CO2 for 48-72 hours.

CD3+ T Cell Activation: T cells were activated by using 1:1 bead:cell ratio of Dynabeads Human T Activator CD3/CD28 beads (Thermofisher) cultured in Immunocult XT media (STEMCELL Technologies) in the presence of 100 U/ml IL-2 (STEMCELL Technologies) and 1×Penicillin/Streptomycin (Thermofisher) at 37 C and 5% CO2 for 48 hours. Post-activation, beads were removed by placement on a magnet and the transfer of the cells back into culture.

Flow cytometry: T cell identity and QC was confirmed by CD3-antibody staining (Biolegend). T cell activation confirmed by CD25 staining Phenotypic Gene KO: TRAC was confirmed by CD3 and TCRab antibody staining (Biolegend); any phenotype data was the percentage change against reference material on viable cells only as ascertained by DAPI staining (BD Bioscience).

Genomic DNA Analysis: Genomic DNA was released from lysed cells 48-72 hours post-electroporation. Loci of interest were amplified by PCR and products then sent for Sanger sequencing. Data was analysed by proprietary in-house software.

### Example 3: Base Editing System Applied to Human Primary Immune Cells Utilizing Synthetic Two-Part crRNA and tracrRNA-Aptamer Guides for Multiplex and Multigene Simultaneous Functional Triplex KO

In this example, primary human Pan T lymphocytes were used to prove the utility of the base editing system for multiplex and multigene KO with crRNA and aptamer modified tracrRNA components in primary immune cells. The Pan T cells were activated utilising anti-CD3 and anti-CD28 and then cells were electroporated with mRNA components for both the deaminase-MCP, nCas9-UGI-UGI components, tracrRNA-Aptamer and crRNAs coding for multiple different genes. The cells were then incubated for a further 72-96 hours and cells were checked for base editing by targeted PCR amplification and Sanger sequencing and the surface KO by a multi-stain panel including (TCRab, B2M and CD52) by flow cytometry; which was used to ascertain multiplex KO in the population.

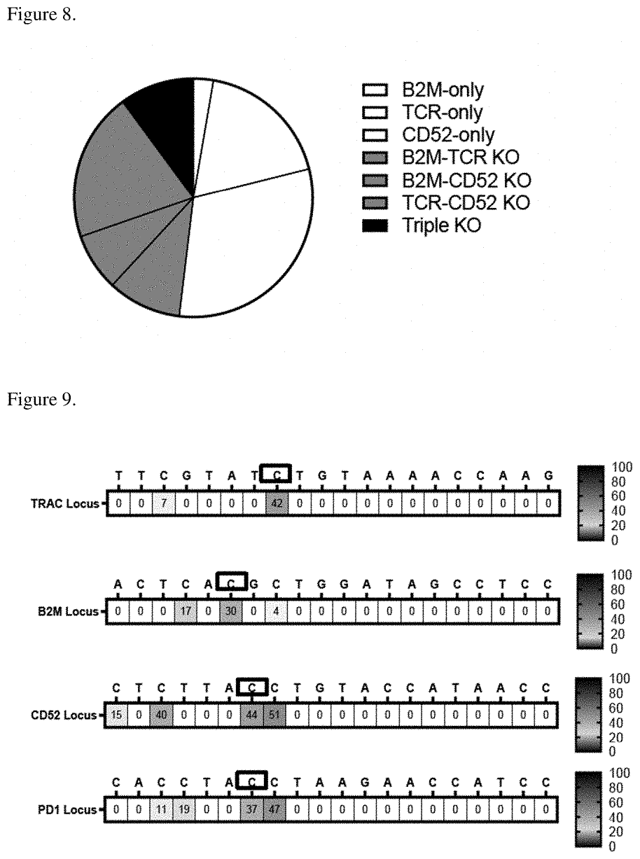

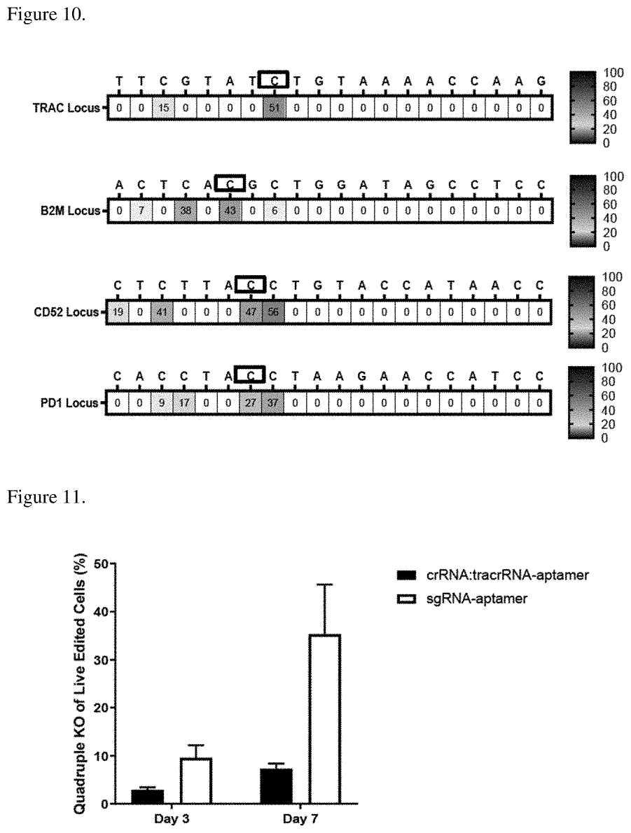

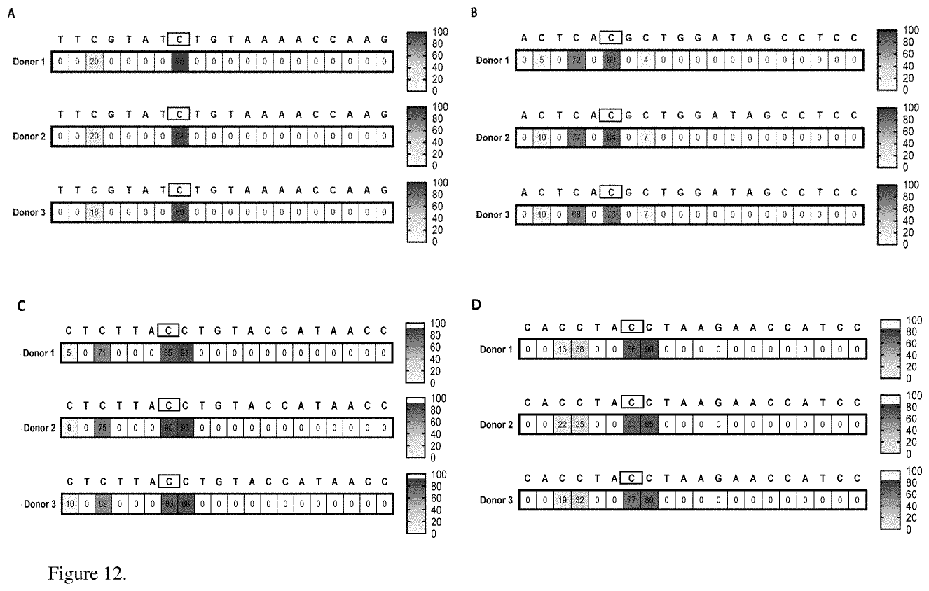

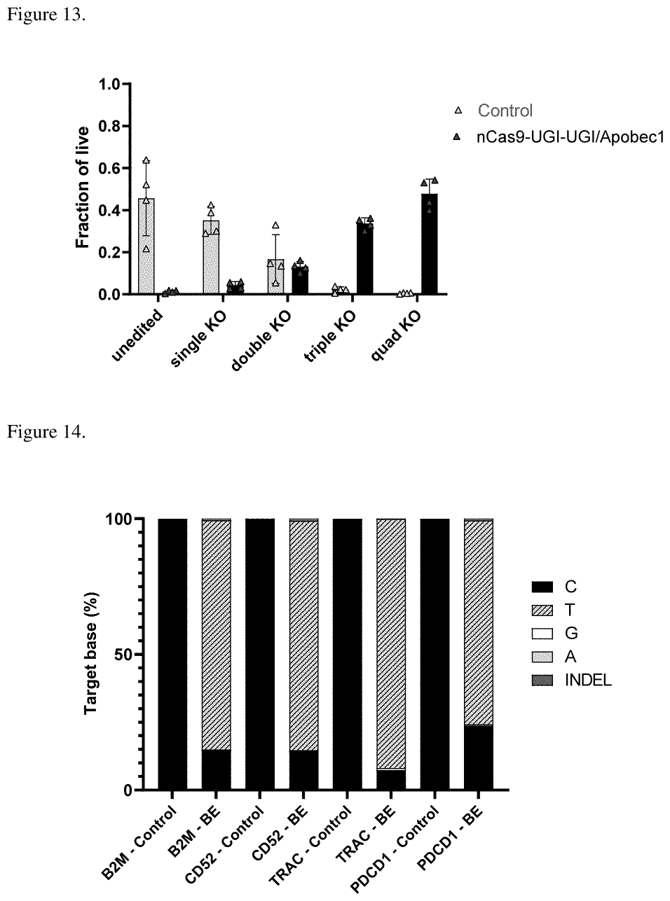

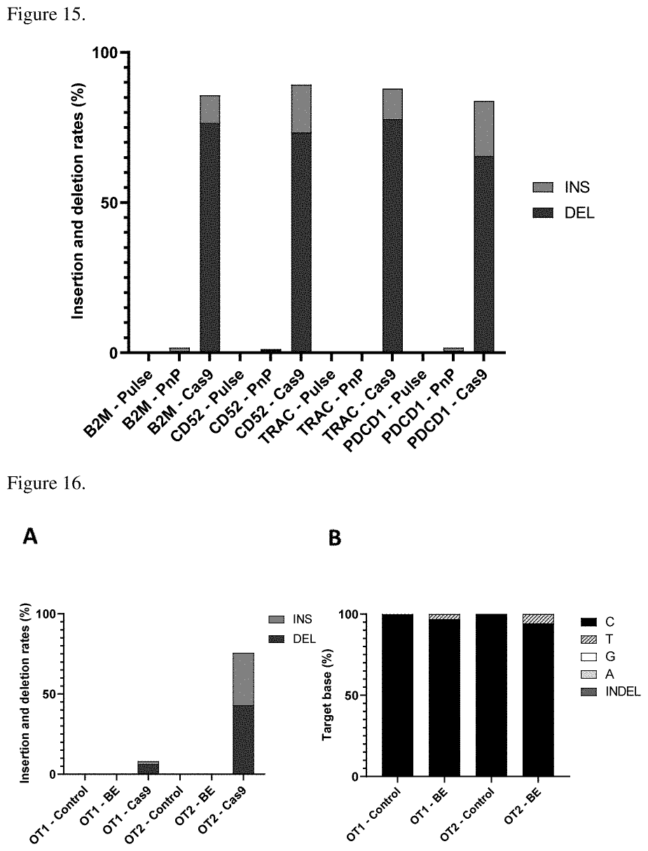

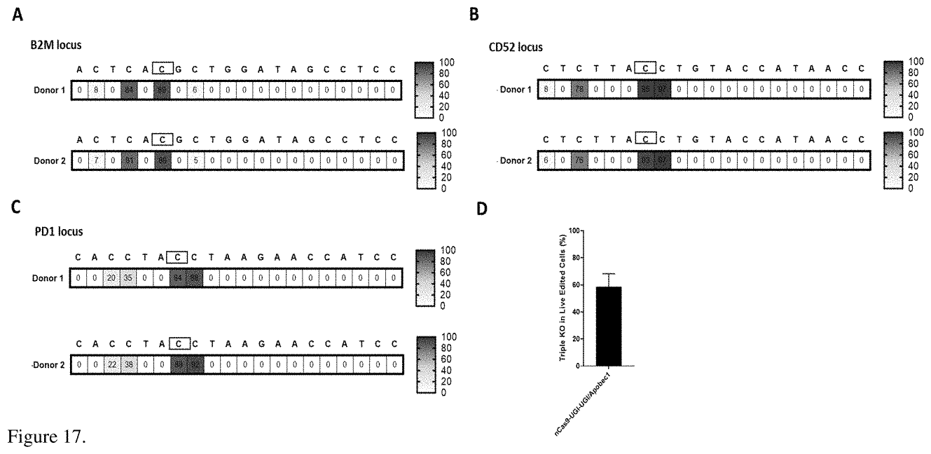

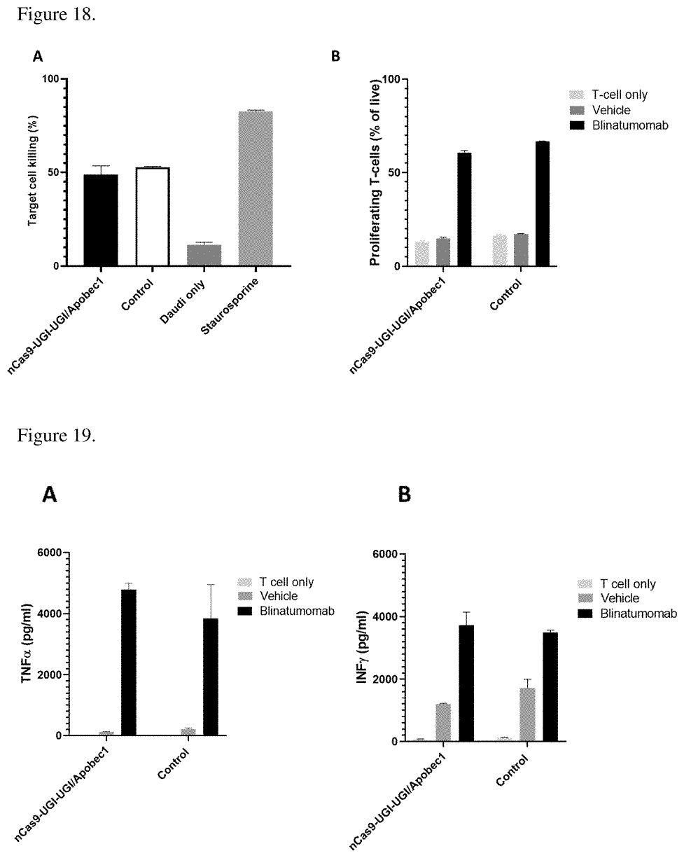

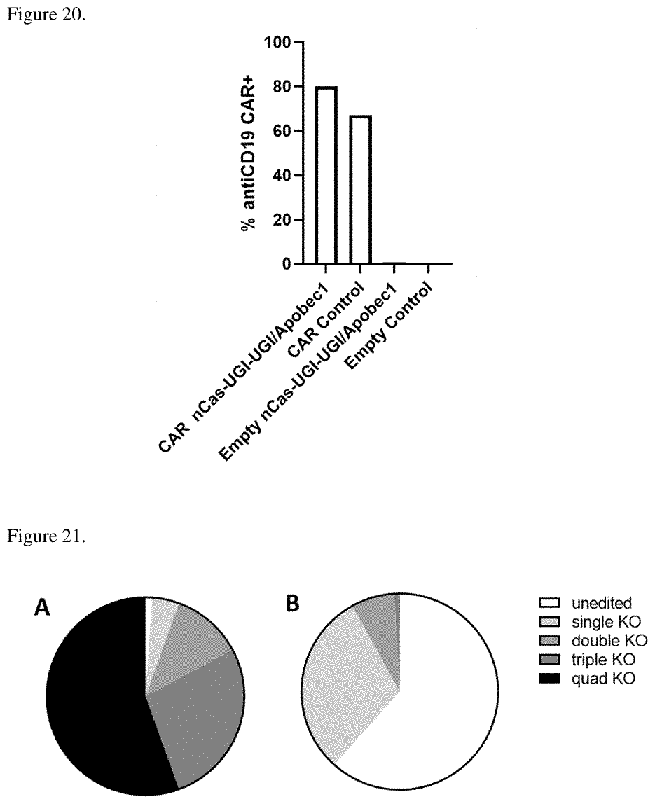

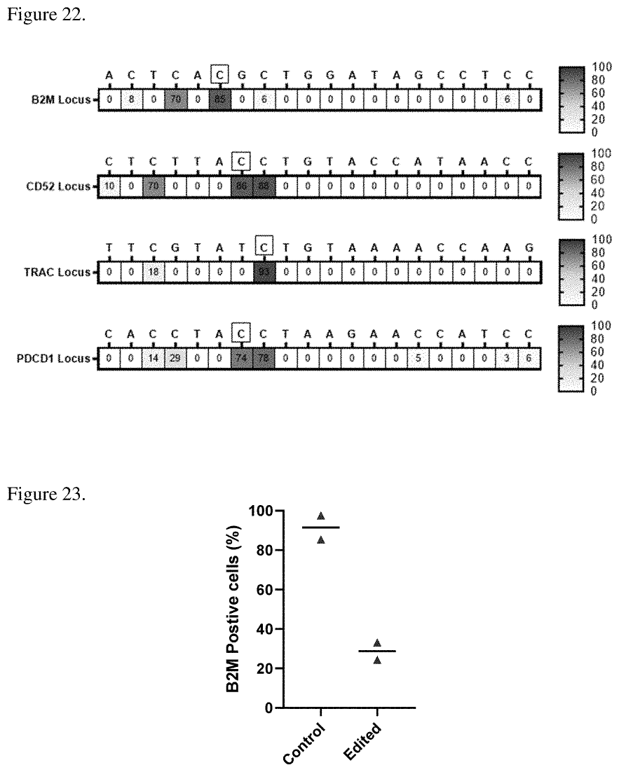

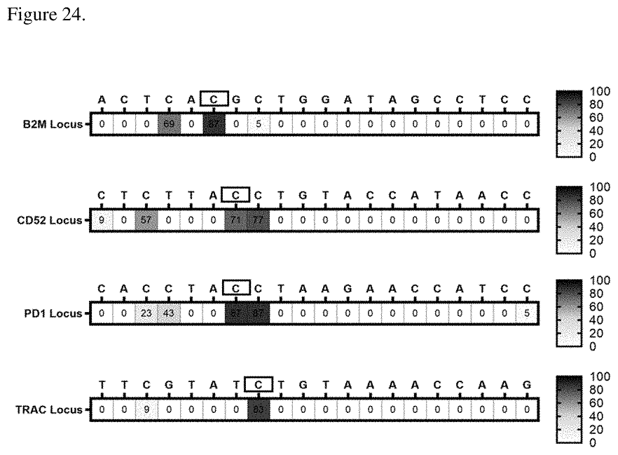

The data shows that this technology can generate high base editing at the DNA level with a good correlation to functional KO information generated by flow cytometry, with a sizeable amount of the population showing triple functional multiplex KO on many genes simultaneously. This displays that the technology has utility simultaneous multigene targeting and can be used to alter many loci in the genome in the same cell; whether for gene KO, correction, attenuation, modification, etc. (FIGS. 7 and 8)

### Example 4: Base Editing System Applied to Human Primary Immune Cells Utilizing Synthetic Two-Part crRNA:tracrRNA-Aptamer and One-Part sgRNA-Aptamer Guides for Multiplex and Multigene Simultaneous Functional Quadruplex KO

In this example, primary human Pan T lymphocytes were used to prove the utility of the base editing system for multiplex and multigene KO with both crRNA and aptamer modified tracrRNA components and sgRNA-aptamer primary immune cells. The Pan T cells were activated utilizing anti-CD3 and anti-CD28 and then cells were electroporated with mRNA components for both the deaminase-MCP, nCas9-UGI-UGI components, and either crRNA:tracrRNA-Aptamer combinations or sgRNA-aptamers for multiple different genes. The cells were then incubated for a further 72-96 hours and cells were checked for base editing by targeted PCR amplification and Sanger sequencing and the surface KO by a multi-stain panel including (TCRab, B2M, CD52, and PD-1) by flow cytometry; which was used to ascertain multiplex KO in the population. Then the remaining cells were incubated for a further 96-120 hours and then the surface KO was studied again by a multi-stain panel including (TCRab, B2M, CD52, and PD-1) by flow cytometry.

The data shows that the technology can generate high base conversion at the DNA level with a good correlation to functional KO information generated by flow cytometry (FIGS. 7 and 8). The flow cytometry data indicates that functional multiplex knock-out by flow cytometry analysis is improved a longer timepoints post-electroporation, which a greater percentage quadruplex found in the Day 7 analysis step when compared to the Day 3 (FIG. 11). Furthermore, the efficacy of the system is surprisingly improved by using one-part sgRNA-aptamers instead of the two-part crRNA:tracrRNA-aptamer systems, with subtle increases in base editing (FIGS. 9 and 10) but a much higher magnitude of of functional quadruplex KO (FIG. 11). This displays additional evidence that the technology has utility for practical simultaneous multigene targeting and can be used to alter many loci in the genome in the same cell; whether for gene KO, correction, attenuation, modification, etc. (FIGS. 9, 10 and 11).

Material and Methods

Guides: Internally generated data was used to specify base editing windows calculated at set distances from the PAM motif (NGG). The data was used to development algorithms to predict Phenotype or Gene KO applicable guides sequence for the following genes: TRAC, TRBC1, TRBC2, PDCD-1, B2M, and CD52 (Table 4). The crRNAs and tracrRNA were synthesized by Horizon Discovery (formerly Dharmacon) and Agilent Technologies. Then selected guides for separate genes were used to prove the utility of the technology.

Selected Synthetic two-part crRNA Sequence for
Multiloci base conversion:
TRAC:
mU*mU*CGUAUCUGUAAAACCAAGGUUUUAGAGCUAUGCUGUUUUG
(SEQ ID NO: 40)
CD52:
mC*mU*CUUACCUGUACCAUAACCGUUUUAGAGCUAUGCUGUUUUG
(SEQ ID NO: 41)
B2M:
mA*mC*UCACGCUGGAUAGCCUCCGUUUUAGAGCUAUGCUGUUUUG
(SEQ ID NO: 42)
PD-1
mC*mA*CCUACCUAAGAACCAUCCGUUUUAGAGCUA UGCUGUUUUG
(SEQ ID NO: 43)
2′OMe (m) and phosphorothioate (*) modified
residues
Synthetic two-part 1xMS2 tracrRNA-Aptamer Sequence
(SEQ ID NO: 44):
AACAGCAUAGCAAGUUAAAAUAAGGCUAGUCCGUUAUCAACUUGAAAAAG
UGGCACCGAGUCGGUGCGCGCACAUGAGGAUCACCCAUGUGCUUUUmU*
mU*U
2′OMe (m) and phosphorothioate (*) modified
residues
Selected Synthetic one-part sgRNA with 1xMS2
tracrRNA-Aptamer Sequence:
TRAC (SEQ ID NO: 45):
mU*mU*CGUAUCUGUAAAACCAAGGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
B2M (SEQ ID NO: 46):
mA*mC*UCACGCUGGAUAGCCUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
CD52 (SEQ ID NO: 47):
mC*mU*CUUACCUGUACCAUAACCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
PD-1 (SEQ ID NO: 48):
mC*mA*CCUACCUAAGAACCAUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
2′OMe (m) and phosphorothioate (*) modified
residues

mRNA Component Generation: Messenger RNA molecules were custom generated by Trilink utilizing modified nucleotides: Pseudouridine and 5-Methyl-Cytosine. The mRNA components translated to the following proteins: Deaminase Apobec1=NLS-rApobec1-Linker-MCP, and Cas9-UGI-UGI=NLS-nCas9-UGI-UGI-NLS

Frozen T Cells Culturing: Sources of frozen CD3+ T Cells (Hemacare) were thawed and then cultured into Immunocult XT media (STEMCELL Technologies) with 1× Penicillin/Streptomycin (Thermofisher) at 37 C and 5% CO2.

CD3+ T Cell Activation: T cells were activated by using 1:1 bead:cell ratio of Dynabeads Human T Activator CD3/CD28 beads (Thermofisher) cultured in Immunocult XT media (STEMCELL Technologies) in the presence of 100 U/ml IL-2 (STEMCELL Technologies) and 1× Penicillin/Streptomycin (Thermofisher) at 37 C and 5% CO2 for 48 hours. Post-activation, beads were removed by placement on a magnet and the transfer of the cells back into culture.

T Cell Electroporation: After 48-72 post-activation T cells were electroporated with using the Neon Electroporator (Thermofisher) or 4D Nucleofector (Lonza). Neon Electroporator conditions were 1600v/10 ms/3 pulses with a 10 ul tip with 250 k cells, combined total of mRNA amount of 1-5 ug, for both the Deaminase-Apobec1 and nCas9-UGI-UGI, and where applicable 0.2-1.8 umol of complexed crRNA:tracrR or sgRNA. Post-electroporation cells were transferred to Immunocult XT media with 100 U IL-2, 100 U IL-7 and 100 U IL-15 (STEMCELL Technologies) and cultured at 37 C and 5% CO2 for 48-72 hours. Media was then changed, cell concentration normalized and incubated for a further 72 hours. Then cells for flow cytometry were stimulated with PMA (50 ng/m) and ionomycin (250 ng/ml) for 48 hours.

Flow cytometry: T cell identity and QC was confirmed by CD3-antibody staining (Biolegend). T cell activation confirmed by CD25 staining Phenotypic Gene Singleplex KO: TRAC was confirmed by CD3 and TCRab antibody staining (Biolegend), B2M by B2M-Antibody (Biolegend), CD52 by CD52-Antibody (Biolegend), and PD-1 by PD-1-Antibody (Biolegend); any phenotype data was the percentage change against reference material on viable cells only as ascertained by DAPI staining (BD Bioscience). Phenotypic Gene Multiplex KO: TRAC was confirmed by TCRab antibody staining (Biolegend), B2M by B2M-Antibody (Biolegend), CD52 with a CD52-antibody (Biolegend), and PD-1-Antibody (Biolegend); any phenotype data was the percentage change against reference material on viable cells only as ascertained by Zombie Yellow staining (Biolegend).

Genomic DNA Analysis: Genomic DNA was released from lysed cells 72-96 hours post-electroporation. Loci of interest were amplified by PCR and products then sent for Sanger sequencing. Data was analyzed by proprietary in-house software.

### Example 5: Base Editing System Applied to Human Primary Immune Cells Utilizing Synthetic One Part sgRNA-Aptamer Guides for Simultaneous Functional Quadruplex KO with Minimal Donor Variation

In this example, primary human Pan T lymphocytes were used to prove the utility and reproducibility of the base editing system for multigene KO with sgRNA-aptamer in primary immune cells. The Pan T cells were activated utilizing anti-CD3 and anti-CD28 and then cells were electroporated with mRNA components for both the deaminase-MCP, nCas9-UGI-UGI components and sgRNA-aptamers for four different genes. The cells were then incubated for 5-7 days and cells were checked for base editing by targeted PCR amplification and Sanger sequencing and the surface KO by a multi-stain panel including (TRACab, B2M, CD52, and PD-1) by flow cytometry, which was used to ascertain multiplex KO in the population.

The data shows that the technology can generate high base conversion at the DNA level with very low variability between multiple donors (FIG. 12). The base conversion has good correlation to functional KO information generated by flow cytometry (FIG. 13). The flow cytometry data indicates high levels of multiple knock-out achieved with the base editing system.

Material and Methods

Guides: Internally generated data was used to specify base editing windows calculated at set distances from the PAM motif (NGG). The data was used to development algorithms to predict Phenotype or Gene KO applicable guides sequence for the following genes: TRAC, TRBC1, TRBC2, PDCD-1, B2M, and CD52 (Table 4). The crRNAs and tracrRNA were synthesized by Horizon Discovery (formerly Dharmacon) and Agilent Technologies. Then selected guides for separate genes were used to prove the utility of the technology.

Selected Synthetic sgRNA with 1xMS2 tracrRNA-
Aptamer Sequence:
TRAC (SEQ ID NO: 45):
mU*mU*CGUAUCUGUAAAACCAAGGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
B2M (SEQ ID NO: 46):
mA*mC*UCACGCUGGAUAGCCUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
CD52 (SEQ ID NO: 47):
mC*mU*CUUACCUGUACCAUAACCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
PD-1 (SEQ ID NO: 48):
mC*mA*CCUACCUAAGAACCAUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
2′OMe (m) and phosphorothioate (*) modified
residues

mRNA Component Generation: Messenger RNA molecules were custom generated by Trilink utilizing modified nucleotides: Pseudouridine and 5-Methyl-Cytosine. The mRNA components translated to the following proteins: Deaminase Apobec1=NLS-rApobec1-Linker-MCP, and Cas9-UGI-UGI=NLS-nCas9-UGI-UGI-NLS

Frozen T Cells Culturing: Sources of frozen CD3+ T Cells (Hemacare) were thawed and then cultured into Immunocult XT media (STEMCELL Technologies) with 1× Penicillin/Streptomycin (Thermofisher) at 37 C and 5% CO2.

CD3+ T Cell Activation: T cells were activated by using 1:1 bead:cell ratio of Dynabeads Human T Activator CD3/CD28 beads (Thermofisher) cultured in Immunocult XT media (STEMCELL Technologies) in the presence of 100 U/ml IL-2 (STEMCELL Technologies) and 1× Penicillin/Streptomycin (Thermofisher) at 37 C and 5% CO2 for 48 hours. Post-activation, beads were removed by placement on a magnet and the transfer of the cells back into culture.

T Cell Electroporation: After 48-72 post-activation T cells were electroporated with using the Neon Electroporator (Thermofisher). Neon Electroporator conditions were 1600v/10 ms/3 pulses with a 10 ul tip with 250 k cells, combined total of mRNA amount of 1-5 ug, for both the Deaminase-MCP and nCas9-UGI-UGI, and where applicable 0.2-1.8 umol of sgRNA. Post-electroporation cells were transferred to Immunocult XT media with 100 U IL-2, 100 U IL-7 and 100 U IL-15 (STEMCELL Technologies) and cultured at 37 C and 5% CO2 for 48-72 hours. Media was then changed, cell concentration normalized and incubated for a further 72 hours. Then cells for flow cytometry were stimulated with PMA (50 ng/ml) and ionomycin (250 ng/ml) for 48 hours.

Flow cytometry: T cell identity and QC was confirmed by CD3-antibody staining (Biolegend). T cell activation confirmed by CD25 staining. Phenotypic Gene Multiplex KO: TRAC was confirmed by TCRab antibody staining (Biolegend), B2M by B2M-Antibody (Biolegend), CD52 with a CD52-antibody (Biolegend), and PD-1-Antibody (Biolegend). Cell populations with single, double, triple, quadruple, or no gene knock-out were identified by flow cytometry performing marker pair-wise analysis. Phenotype data is reported as fraction of live cells.

Genomic DNA Analysis: Genomic DNA was released from lysed cells 5-7 days post-electroporation. Loci of interest were amplified by PCR and products then sent for Sanger sequencing (Genewiz). Data was analyzed by proprietary in-house software.

### Example 6. Comparison of Base Editing Technology to Nuclease Active Cas9 Technology at on and Off-Targets by Next Generation Sequencing

In this example, primary human T cells were used to demonstrate successful on target genomic DNA (gDNA) editing at four therapeutically relevant loci, specifically B2M, CD52, TRAC and PDCD1, in response to APOBEC1 deaminase-based editor (quadruple knockout, multiplex format). FIG. 14 shows nucleotide conversion rates at the target C that allows for disruption of a splice acceptor site (TRAC) or splice donor site (B2M, CD52, PDCD1), ultimately enabling genetic knockout of the relevant gene. Data in the figure indicate that we obtain high C-to-T editing at the target nucleotide in response to base editor, and also highlight the high level of purity with regards to base conversion. The latter is apparent from minimal non-C-to-T editing (C-to-G or C-to-A conversion), as well as barely detectable indel formation. FIG. 15 shows total rates of insertions and deletions across the 20 bp protospacer in response to base editor or SpCas9 targeting the four genes B2M, CD52, TRAC and PDCD1. Results demonstrate that SpCas9 confers high levels of indel formation at the target loci whilst base editor does not induce this form of editing, suggesting that the latter does not facilitate double strand breaks in the DNA to facilitate gene knockout. In the final section of this example, genomic editing at two computationally predicted sgRNA-dependent off target sites was evaluated for indel formation (FIG. 16A) and base conversion (FIG. 16B). The two sites were previously identified as edited in a screen of forty predicted off target sites (ten per target-specific sgRNA; screen performed internally) and were carried through to subsequent experiments and safety profiling of gene editing reagents. Results in FIG. 16A illustrate that SpCas9 induces high levels of insertions and deletions at both the B2M sgRNA-associated off target site (OT1) and the PDCD1 sgRNA-associated off target site (OT2), whilst base editor does not induce this form of gene editing. Results in FIG. 16B show C-to-T conversion at the two predicted off target sites in response to base editor; however, the proportion and level of genomic editing is much lower compared with SpCas9-induced indel formation. All figures were generated from using the mean of n=3 biological replicates from three independent experiments performed by three different operators. Data were compiled from two next generation sequencing (NGS) runs performed on a MiSeq system.

In silico prediction of sgRNA-dependent off target sites. Lists of predicted off target sites were generated for B2M-, CD52-, TRAC- and PDCD1-targeting sgRNA using a combination of publicly available computational tools (CRISPOR, CCTop, Cas-OFFinder, WGS) and a proprietary in-house computational tool. Input criteria of the former tools focused on mismatches whilst the internal tool focused on gaps and bulges. 10 predicted off target sites per on target sgRNA were selected for genomic analysis of predicted off target editing, therefore a total of 40 off target sites were evaluated per gDNA sample. Two out of 40 evaluated off target sites were positive for editing in the screen therefore were retained in all subsequent experiments. These sites correspond to a B2M sgRNA-dependent off target site and a PDCD1 sgRNA-dependent off target site which are referred to as OT1 and OT2, respectively.

gDNA extraction. Genomic DNA was extracted from human primary T cells 7 days post-electroporation. Briefly, cells were pelleted at 500 G for 5 min. and resuspended in 20-120 μL DirectPCR (cell) lysis buffer [Viagen #302-C] containing Proteinase K [Sigma] to a final concentration of 2.5E3 cells/μL. gDNA was released from cells under the following conditions: 55° C. for 30 min., 95° C. for 30 min. and final cool down to 4° C. in a thermal cycler. gDNA lysates were stored at −20° C. until ready for downstream PCR.

Alternatively, gDNA was extracted using a column-based method using a DNeasy Blood & Tissue Kit [Qiagen] according to the manufacturer's instructions. Purified gDNA was eluted in 30 μL UtraPure H2O, quantified using a NanoDrop spectrophotometer [ThermoScientific] and diluted to 100 ng/μL for downstream PCR (25 ng per 25 μl PCR reaction).

PCR amplification. Primers were designed to span the on target and off target loci of interest (Table 4). An Illumina adapter and linker sequence were appended to target-specific primers for downstream barcoding.

TABLE 4

| Target | Chromosomal | | | Amplicon |
|---|---|---|---|---|
| locus | coordinates | Forward primer | Reverse primer | (bp) |
| B2M | chr15: 44711600- | TGACTGGAGTTCAGA | ACACTCTTTCCCTAC | 313 |
| | 44711619 | CGTGTGctcttccgatctGG | ACGACGctcttccgatctC | |
| | | CCTTGTCCTGATTGGC | GCTTCCCCGAGATC | |
| | | TG (SEQ ID NO: 49) | CAG (SEQ ID NO: 50) | |
| CD52 | chr1: 26318059- | TGACTGGAGTTCAGA | ACACTCTTTCCCTAC | 313 |
| | 26318078 | CGTGTGctcttccgatctAA | ACGACGctcttccgatctC | |
| | | GCTGCTACCAAGACA | AGGTTTCTCTCAGG | |
| | | GCC (SEQ ID NO: 51) | GCAGC (SEQ ID NO: | |
| | | | 52) | |
| TRAC | chr14: 22550544- | TGACTGGAGTTCAGA | ACACTCTTTCCCTAC | 336 |
| | 22550563 | CGTGTGctcttccgatctGG | ACGACGctcttccgatctC | |
| | | GGATATGCACAGAAG | TCAGAGCTTAGGAT | |
| | | CTGC (SEQ ID NO: 53) | GCACCC (SEQ ID NO: | |
| | | | 54) | |
| PDCD1 | chr2: 241858756- | TGACTGGAGTTCAGA | ACACTCTTTCCCTAC | 311 |
| | 241858775 | CGTGTGctcttccgatctGG | ACGACGctcttccgatctC | |
| | | CACCCTCCCTTCAAC | TCCAGACCCCTCGC | |
| | | CT (SEQ ID NO: 55) | TCC (SEQ ID NO: 56) | |
| OT1 | chr20: 22332541- | ACACTCTTTCCCTACA | TGACTGGAGTTCAG | 273 |
| | 22332561 | CGACGctcttccgatctTGC | ACGTGTGctcttccgatct | |
| | | CTTCCCAGACCCAGA | AGATCAGGCTGGCG | |
| | | (SEQ ID NO: 57) | GGC (SEQ ID NO: 58) | |
| OT2 | chr15: 92446128- | TGACTGGAGTTCAGA | ACACTCTTTCCCTAC | 308 |
| | 92446147 | CGTGTGctcttccgatctCAT | ACGACGctcttccgatctA | |
| | | GATAGCAGCCACAAG | GCCCATGCGGAGAG | |
| | | AA (SEQ ID NO: 59) | ATCAT (SEQ ID NO: | |
| | | | 60) | |

Bioinformatics NGS data processing and analysis of base editing and indel formation. High throughput sequencing data analysis, specifically frequency of single nucleotide polymorphisms (SNPs) and insertions/deletions (indels), was performed as follows: paired-end reads were merged using a custom Python script, which filters out any reads with mismatches in the overlapping region and keeps the higher Phred score for each overlapping base. The non-overlapping portions of the reads were then trimmed off and merged reads containing any base with a Phred score <30 were filtered out. The resulting reads were aligned using Bowtie2 (Langmead B, Salzberg SL. Fast gapped-read alignment with Bowtie 2. Nature methods. 2012; 9:357-359. DOI: 10.1038/nmeth.1923) and a mpileup file was generated using SAMtools (Li H, Handsaker B, Wysoker A et al. The Sequence Alignment/Map format and SAMtools. Bioinformatics. 2009; 25:2078-2079. DOI: 10.1093/bioinformatics/btp352).

### Example 7: Functional Readouts on Edited Human Primary Immune Cells Utilizing Synthetic One-Part sgRNA-Aptamer Guides for Multiplex Simultaneous Functional Triplex KO

In this example, multiplex edited primary human Pan T lymphocytes were used in a co-culture assay with target tumor cells to assess whether the edited cells were still functional. In the assay the ability of the T cells to kill tumor cells, to proliferate and to release cytokines were assessed and found that unedited and edited cells function as expected, which shows that the base editing system does not affect cell functionality.

The Pan T cells were activated utilizing anti-CD3 and anti-CD28 and then cells were electroporated with mRNA components for both the deaminase-MCP, nCas9-UGI-UGI components and sgRNA-aptamers for three different genes. The cells were then incubated for 5-7 days and cells were checked for base editing by targeted PCR amplification and Sanger sequencing (FIGS. 17A,B and C) and the surface KO by a multi-stain panel including (B2M, CD52, and PD-1) by flow cytometry, which was used to ascertain multiplex KO in the population (FIG. 17 D).

The triplex KO, and an unedited, T cell populations were then used in killing assays against cells that presented the CD19 surface antigen (Daudi; a B cell cancer line). To facilitate cell killing, a bispecific antibody was added that facilitates the connection between the T cell receptor (TCR) and the CD19 surface antigen (bispecific used was Blinatumomab). The assay was run with several controls, including that of T cells only (without any additional tumor cells), vehicle (which is the buffer used for the bispecific antibody, but not antibody present), Daudi only (without T cells) to assess base line viability of the target cells and Staurosporine (Daudi cells treated with the molecule) to assess maximal target cells death. The assay was run over several days, and at the end point the cells were assessed to determine if they are functionally normal. Three read-outs were examined: T cells killing ability (lysis of target cells by flow cytometry), T cells ability to proliferate (by flow cytometry) and to release cytokines (by flow cytometry) because of interacting with the target tumor cell. The tumor cell killing assay (FIG. 18) showed that both positive (total lysis conditions) and negative (Daudi only) controls of the assays were good and that the edited and unedited cells could both kill tumor cells with good efficiency, with no evidence diminished activity to that of unedited T cells (FIG. 18A). Additionally, assessment of T cell proliferation was determined by CellTrace CSFE staining and flow cytometry showing that the edited and unedited T cells both reacted to the tumor cell killing in the same way with no difference observed (FIG. 18B). The final method to assess functionality was that of testing the ability of the cells to release cytokines (specifically tumor necrosis factor alpha (TNFa) and interferon gamma (IFNg)) from the supernatant of the T cell killing assay (FIG. 19). As expected, the edited and unedited cells behaved consistently with no significant differences observed in the amount of cytokine released. In conclusion, all three assays indicate that the application of the exemplified base editing technology can be applied in T cells without effecting therapeutically relevant functionality.

Material and Methods

Guides: Internally validated synthetic sgRNAs for the following genes were used to induce knock-out: PDCD-1, B2M, and CD52. The sgRNAs were synthesized by Agilent Technologies.

Selected Synthetic sgRNA with 1xMS2 tracrRNA-
Aptamer Sequence:
B2M (SEQ ID NO: 46):
mA*mC*UCACGCUGGAUAGCCUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
CD52 (SEQ ID NO: 47):
mC*mU*CUUACCUGUACCAUAACCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
PD-1 (SEQ ID NO: 48):
mC*mA*CCUACCUAAGAACCAUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
2′OMe (m) and phosphorothioate (*) modified
residues

mRNA Component Generation: Messenger RNA molecules were custom generated by Trilink utilizing modified nucleotides: Pseudouridine and 5-Methyl-Cytosine. The mRNA components translated to the following proteins: Deaminase Apobec1=NLS-rApobec1-Linker-MCP, and Cas9-UGI-UGI=NLS-nCas9-UGI-UGI-NLS

Frozen T Cells Culturing: Sources of frozen CD3+ T Cells (Hemacare) were thawed and then cultured into Immunocult XT media (STEMCELL Technologies) with 1× Penicillin/Streptomycin (Thermofisher) at 37 C and 5% CO2.

CD3+ T Cell Activation: T cells were activated by using 1:1 bead:cell ratio of Dynabeads Human T Activator CD3/CD28 beads (Thermofisher) cultured in Immunocult XT media (STEMCELL Technologies) in the presence of 100 U/ml IL-2 (STEMCELL Technologies) and 1× Penicillin/Streptomycin (Thermofisher) at 37 C and 5% CO2 for 48 hours. Post-activation, beads were removed by placement on a magnet and the transfer of the cells back into culture.

T Cell Electroporation: After 48-72 post-activation T cells were electroporated using the Neon Electroporator (Thermofisher). Neon Electroporator conditions were 1600v/10 ms/3 pulses with a 10 ul tip with 250 k cells, combined total of mRNA amount of 1-5 ug, for both the Deaminase-MCP and nCas9-UGI-UGI, and where applicable 0.2-1.8 umol of sgRNA. Post-electroporation cells were transferred to Immunocult XT media with 100 U IL-2, 100 U IL-7 and 100 U IL-15 (STEMCELL Technologies) and cultured at 37 C and 5% CO2 for 48-72 hours. Media was then changed, cell concentration normalized, and incubated for a further 72 hours.

Flow cytometry: T cell identity and QC was confirmed by CD3-antibody staining (Biolegend). T cell activation confirmed by CD25 staining. Phenotypic Gene Multiplex KO: B2M was confirmed by B2M-Antibody staining (Biolegend), CD52 with a CD52-antibody (Biolegend), and PD-1-Antibody (Biolegend). Any phenotype data was the percentage change against reference material on viable cells only as ascertained by Zombie Yellow staining (Biolegend).

Genomic DNA Analysis: Genomic DNA was released from lysed cells 5-7 days post-electroporation. Loci of interest were amplified by PCR and products then sent for Sanger sequencing (Genewiz). Data was analyzed by proprietary in-house software.

Target-cell Killing assay: Target-cell killing assay was set-up 7 days post cells electroporation. The assay was performed in RMPI media (Thermofisher) supplemented with 10% FBS (Thermofisher) and 100 U/ml IL-2 (STEMCELL Technologies). T cells (effector) were co-cultured with Daudi cells (target) at a 4:1 effector:target ratio. Daudi cells were seeded in a 96-well plate (50000 per well) with or without the bispecific antibody Blinatumomab (10 nM). To assess T cells proliferation, T cells were stained with the CellTrace CSFE (Thermofisher) before the assay. As negative control, basal target cell viability of Daudi cells was measured in absence of T cells. As positive control, maximum target cell death was induced by staurosporine treatment of Daudi cells. After three days of co-culture, T cells proliferation and target cell killing were tested by Flow cytometry. Cells were stained with a CD20 antibody (Biolegend) to identify Daudi cells and with a CD2 antibody (Biolegend) to identify T cells.

Cytokines (TNFa and INFg) were measured in the supernatant collected after 72 h of co-culture using the MultiCyt Qbeads Plex Screen Assay (Sartorius) following the manufacturer instructions. In this assay beads coated with capture antibodies are mixed with cell culture supernatant. The analyte of interest binds to the antibodies on the beads. A fluorescent detection antibody is added which binds to the analyte captured on the bead and generates a fluorescent signal which is directly proportional to the concentration of the analyte in the cell culture supernatant. The beads are washed to improve sensitivity of the assay and data acquired using an IQue Screener PLUS flow cytometer. Standard curves were generated using the iQue ForeCyt® Enterprise Client Edition 8.1 (R3) software and used to calculate the absolution concentration of INF-γ and TNF-α.

### Example 8: Base Editing System Applied in Lentiviral Transduced Human Primary Immune Cells Utilizing Synthetic One-Part sgRNA-Aptamer Guides for Simultaneous Functional Quadruplex KO

In this example, human Pan T lymphocytes were transduced using a lentiviral cassette and then selected by antibiotics before utilizing the base editing system for multiplex gene editing. The viral cassette contained two genes: puromycin and anti-CD19 chimeric antigen receptor (CAR). A total of four genes were targeted base editing, and functional KO: B2M, CD52, TRAC, and PD1. The examples displays that T cells be readily transduced and base edited after the event with high efficiency.

The Pan T cells were isolated and then activated utilizing anti-CD3 and anti-CD28 antibodies so that the cells can be transduced efficiency. The T cells were the transduced using in-house prepared viral stocks of high titre, using Rectronectin to aid delivery. The transduced cells were then selected using the antibiotic puromycin to ensure the population contained the lentiviral cassette. The T cells were then allowed to recover, re-activated utilizing anti-CD3 and anti-CD28 and then cells were electroporated with mRNA components for both the deaminase-MCP, nCas9-UGI-UGI components, sgRNA-Aptamer coding for multiple different genes. The cells were then incubated for a further 72-96 hours, and then stimulated with PMA and ionomycin for 48 hours. The cells were then checked for base editing by targeted PCR amplification and Sanger sequencing and the surface KO by a multi-stain panel including (AntiCD19 CAR, TRACab, B2M, CD52, and PD1) by flow cytometry (using DAPI as a viability dye); which was used to ascertain multiplex KO in the population. The flow cytometry data display high transduction efficient (FIG. 20) and then base editing was readily applied to the transduced cells with surprisingly high efficiency (FIG. 21) with the majority of the population being CAR positive and displaying quadruplex KO. Additionally, the DNA corroborates with the flow cytometry data with high on target base editing. This exemplifies the utility of the base editing technology on transduced primary cell populations, which is clinically relevant for CAR and other technology for engineering immune cells for improved functionality.

Material and Methods

Guides: Internally generated data was used to specify base editing windows calculated at set distances from the PAM motif (NGG). The data was used to development algorithms to predict Phenotype or Gene KO applicable guides sequence for the following genes: TRAC, TRBC1, TRBC2, PDCD-1, B2M, and CD52 (Table 4). The sgRNAs were synthesized by Agilent Technologies. Then selected guides for separate genes were used to prove the utility of the technology.

Selected Synthetic sgRNA with 1xMS2 tracrRNA-
Aptamer Sequence:
TRAC (SEQ ID NO: 45):
mU*mU*CGUAUCUGUAAAACCAAGGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
B2M (SEQ ID NO: 46):
mA*mC*UCACGCUGGAUAGCCUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
CD52 (SEQ ID NO: 47):
mC*mU*CUUACCUGUACCAUAACCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
PD-1 (SEQ ID NO: 48):
mC*mA*CCUACCUAAGAACCAUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
2′OMe (m) and phosphorothioate (*) modified
residues

mRNA Component Generation: Messenger RNA molecules were custom generated by Trilink utilizing modified nucleotides: Pseudouridine and 5-Methyl-Cytosine. The mRNA components translated to the following proteins: Deaminase Apobec1=NLS-rApobec1-Linker-MCP, and nCas9-UGI-UGI=NLS-nCas9-UGI-UGI-NLS

Pre-Transduction T cell activation. Pan T cells were isolated from whole blood (Cambridge Bioscience) using the EasySep™ human T cell isolation kit (StemCell™ Technologies) and cryopreserved until required. Frozen Pan T cells were thawed and cultured in ImmunoCult™-XV T cell expansion medium (StemCell™ Technologies) supplemented with human serum (Gibco), penicillin-streptomycin (Gibco), and recombinant interleukin-2 (Miltenyi Biotec). Cells were activated on anti-human CD3 (Biolegend) coated plates in the presence of soluble anti-human CD28 (Biolegend) and incubated at 37° C. and 5% CO2 overnight.

Lentivirus transduction. Two different lentiviral vectors both carrying a puromycin resistance gene cassette were used. The experimental lentivirus additionally contained a gene to confer antiCD19 CAR expression on the experimental cells (CAR), while the control vector did not (EMPTY). Non-TC treated multi well plates were coated with RetroNectin® Recombinant Human Fibronectin Fragment (Takara). Lentivirus was centrifuged onto RetroNectin® (Takara)-coated plates, then the activated T cells were added. Cells were incubated at 37° C. and 5% CO2 to allow the transduction to take place.

Selection and expansion. After transduction, cells were replated in ImmunoCult™-XV T cell expansion medium (StemCell™ Technologies) supplemented with human serum (Gibco), penicillin-streptomycin (Gibco), recombinant interleukin-2 (Miltenyi Biotec), ImmunoCult™ human CD3/CD28 T cell activator (StemCell™ Technologies) and puromycin (Gibco). This cocktail allowed expansion of the virally transduced cells with simultaneous elimination of the non-transduced cells which were sensitive to puromycin treatment.

CD3+ T Cell Activation: The selected transduced population of T cells was reactivated using 1:1 bead:cell ratio of Dynabeads Human T Activator CD3/CD28 beads (Thermofisher) cultured in ImmunoCult™-XV T cell expansion medium (StemCell™ Technologies) supplemented with 100 U/ml IL-2 (StemCell™ Technologies) and 1× penicillin-streptomycin (Gibco) at 37° C. and 5% CO2 for 48 hours. Post-activation, beads were removed by magnetic separation and the cells transferred back into culture.

T Cell Electroporation: On the day of bead removal, activated T cells were electroporated using the Neon Electroporator (Thermofisher). Neon Electroporation conditions were 1600v/10 ms/3 pulses with a 10 μl tip. Approximately 200,000 cells, 2 ng combined total of mRNA (both the Deaminase-Apobec1 and nCas9-UGI-UGI), and 2 μM of each sgRNA were used for the quadruple KO, while 200′000 cells without mRNA or sgRNA was used as a control. Post-electroporation cells were cultured in ImmunoCult™-XV T cell expansion medium (StemCell™ Technologies) supplemented with 100 U IL-2, 100 U IL-7 and 100 U IL-15 (StemCell™ Technologies) and cultured at 37° C. and 5% CO2. Cell numbers were standardized and growth medium replenished when necessary. At day 5 post electroporation, T cells were restimulated with PMA (50 ng/ml) and ionomycin (250 ng/ml).

Phenotypic analysis. Surface expression of the antiCD19 CAR was confirmed in the same flow cytometry staining panel which was used to assess phenotypic KO of B2M, CD52, TCR and PD1. This enabled the quantification of target knockouts within the antiCD19 CAR+ population of cells. Antibodies were supplied by Biolegend (anti-B2M, anti-TCR and anti-PD1), BD BioScience (anti-CD52) and AcroBiosystems (anti-FMC63 scFv antibody for the identification of CD19-specific CAR+ T cells). Data were acquired on an iQue Screener PLUS Flow cytometer and analyzed using iQue ForeCyt® Enterprise Client Edition 8.1 (R3) software. Live antiCD19 CAR positive cell populations with single, double, triple, quadruple, or no gene knock-out were identified by performing marker pair-wise analysis on the live antiCD19 positive population. Phenotype data is reported as fraction of CAR+ live cells.

Genomic DNA Analysis. Genomic DNA was released from lysed cells 7 days post-electroporation. Loci of interest were amplified by PCR and products then sent for Sanger sequencing (Genewiz). Data was analyzed by proprietary in-house software (FIG. 22).

### Example 9: Base Editing System Applied to Human Induced Pluripotent Stem Cells Utilising Synthetic One Part sgRNA-Aptamer Guides

In this example, iPSCs were used to prove the utility of the base editing system for singleplex editing using the single piece sgRNA form with an RNA aptamer present. The iPSCs were cultivated in line specific media and then cells were electroporated with mRNA components for both the deaminase-MCP, nCas9-UGI-UGI components, sgRNA-aptamer coding for the B2M gene. The cells were then incubated for a further 72-96 hours and cells were checked for base editing by surface KO by a B2M-staining and flow cytometry.

The data shows that this base editing technology can generate high base editing that is effective at disrupting the B2M and leading to a strong functional KO, as exemplified by the large drop in B2Ms presence on the surface compared to the control (FIG. 23). Exemplifying that the technology is not limited in scope to primary cells but can be used for cells of a stem cell identity.

### Example 10: Base Editing System Applied to Human Primary Immune Cells Utilizing Synthetic One Part sgRNA-Aptamer Guides for Multiplex Simultaneous Quadruplex KO

In this example, iPSCs were used to prove the utility of the base editing system for multiplex editing using the single piece sgRNA form with an RNA aptamer present. The iPSCs were cultivated in line specific media and then cells were electroporated with mRNA components for both the deaminase-MCP, nCas9-UGI-UGI components, sgRNA-aptamers coding for genes: B2M, CD52, TRAC, and PDCD1. The cells were then incubated for a further 72-96 hours and cells were checked for base editing by targeted PCR amplification and Sanger sequencing (FIG. 24). Thereby exemplifying that the base editing technology can be readily utilized for multiplex editing in iPSCs, and again displaying that the technology can be used for cells of a stem cell identity.

Material and Methods

Guides: Internally generated data was used to determine the position of base editing windows in relation to Cas9 PAM motifs (NGG). The data was used to develop algorithms to predict guide sequences applicable to gene disruption through the generation of premature STOP codons or the modification of splice donor/acceptor sites. The following genes were simultaneously disrupted using single-guide RNAs (sgRNAs) synthesized by Agilent Technologies: TRAC, PDCD-1, B2M, and CD52 (See below).

Selected Synthetic sgRNA with 1xMS2 tracrRNA-
Aptamer Sequence:
TRAC (SEQ ID NO: 45):
mU*mU*CGUAUCUGUAAAACCAAGGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
B2M (SEQ ID NO: 46):
mA*mC*UCACGCUGGAUAGCCUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
CD52 (SEQ ID NO: 47):
mC*mU*CUUACCUGUACCAUAACCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
PD-1 (SEQ ID NO: 48):
mC*mA*CCUACCUAAGAACCAUCCGUUUUAGAGCUAGAAAUAGCAAGUUA
AAAUAAGGCUAGUCCGUUAUCAACUUGAAAAAGUGGCACCGAGUCGGUGC
GCGCACAUGAGGAUCACCCAUGUGCUUUUmU*mU*U
2′OMe (m) and phosphorothioate (*) modified
residues

mRNA Component Generation: Messenger RNA molecules encoding NLS-rApobec1-Linker-MCP (Deaminase-MCP) and NLS-nCas9-UGI-UGI-NLS (nCas9-UGI) proteins were custom generated by Trilink utilizing modified nucleotides (Pseudouridine and 5-Methyl-Cytosine).

hiPSC Culture: Frozen hiPSCs were obtained from multiple independent sources (ThermoFisher Scientific (Gibco line), Coriell institute (WTC-11 line)). Cells were thawed and cultured in mTesr-PLUS medium (STEMCELL Technologies) at 37 C and 5% CO2. The Gibco hiPSC line was cultured on Vitronectin-XF (STEMCELL Technologies) coated non-adherent cell-culture plasticware (Greiner Bio-One) and passaged in clumps using Versene dissociation reagent (ThermoFisher Scientific). The WTC-11 hiPSC line was cultured on Geltrex (ThermoFisher Scientific) coated cell-culture plasticware (Corning), and passaged as single-cells using Accutase dissociation reagent (ThermoFisher Scientific), with the inclusion of 10 μM of the Rho-kinase inhibitor Y-27632 (STEMCELL Technologies) in culture medium for 24 hrs post-passage to promote cell survival. Medium was exchanged at 1-3 day intervals and cells were passaged at 3-5 day intervals as required.

hiPSC Electroporation: 2-4 hrs prior to electroporation hiPSCs were fed with fresh mTesr-PLUS culture medium (STEMCELL Technologies) containing 10 μM Y-27632 (STEMCELL Technologies), then dissociated to single cells using Accutase (ThermoFisher Scientific). 200 k-300 k cells were resuspended in 20 ul of Buffer P3 (Lonza) and combined with 1-4 ug of modified mRNA (Trilink) encoding Deaminase-MCP and nCas9-UGI proteins, and 1-4 μM of each sgRNA (Agilent). Electroporation was performed with the 4D Nucleofector (Lonza) in a 16×20 μl multi-well cuvette using programmes CM138 or CB150. Post-electroporation, cells were seeded on Geltrex (ThermoFisher Scientific) coated cell-culture plasticware (Corning) in mTesr-PLUS (STEMCELL Technologies) with the inclusion of 10 μM of the Rho-kinase inhibitor Y-27632 (STEMCELL Technologies) in culture medium for 24 hrs post-electroporation to promote cell survival.

Post-electroporation QC: Maintenance of hiPSC identity post-electroporation was confirmed by OCT4 and Nanog immunostaining (BD Bioscience) and flow-cytometry analysis.

Singleplex KO: Disruption of B2M protein expression was analysed by B2M-Antibody (Biolegend) immunostaining and flow-cytometry, and quantified by determining B2M expression at 72-96 hours; displaying the viable cells (DAPI negative) as edited (with sgRNA) and control (no sgRNA present) electroporated samples.

Multiplex KO: Cells were lysed 96-120 hours post-electroporation. sgRNA-targeted gene loci were amplified by PCR from genomic DNA and Sanger sequenced (Genewiz). Data was analyzed by proprietary in-house software.

### CONCLUSION

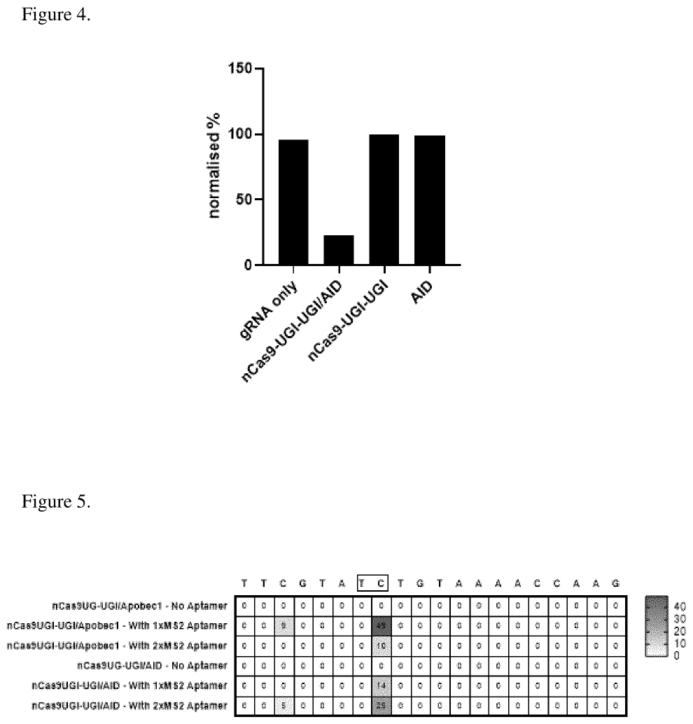

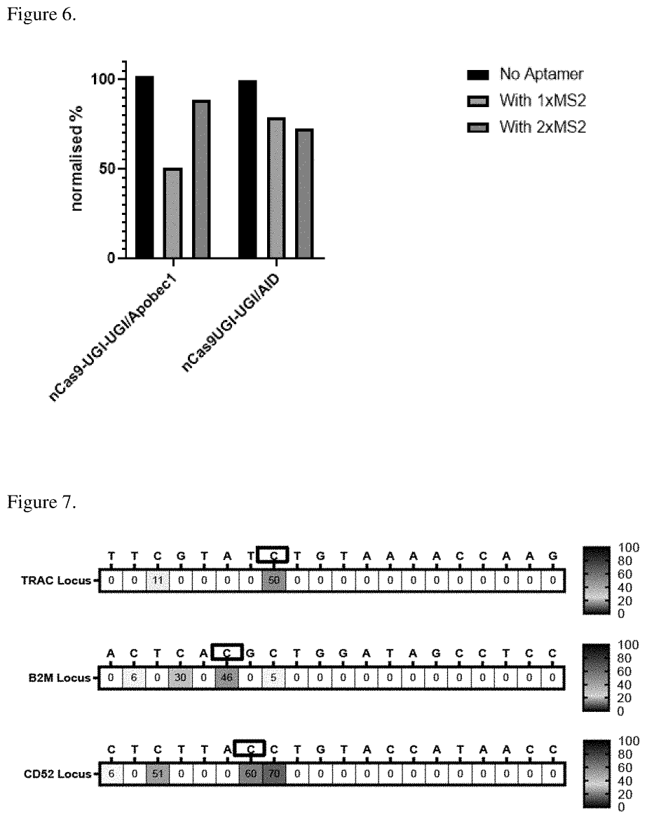

The data exemplified display that an RNA mediated base editing system is a versatile system that can be readily used in immune cells, including that of primary human cells (FIGS. 1-22) and that of human stem cells (FIGS. 23-24). The modular base editing system has been displayed by its ability to switch elements in and out to create differing editing profiles based on aptamer and deaminase selection (FIG. 5). This effect is key to the core value of RNA mediated base editing technology, and showcases the high utility of the modular system to be tailored to therapeutically relevant treatment routes depending on the desired phenotypic outcome.

In this body of work, it has been displayed that RNA mediated base editing system is compatible with multiple different forms of guide delivery, including stable expression (by lentiviral integration and exogenous expression under the human U6 PolII promoter) (FIG. 2,3,4), by a two-part crRNA-tracrRNA synthetic guide (FIG. 5,6,7,8,10), and by a single part crRNA-tracrRNA synthetic guide (9,10,11,12,13). The inclusion into the genome by lentiviral integration is an atypical therapeutic root, but does display that the technology can be readily used for applications where the guide integration is important (e.g. Pooled genetic screens) and it also showcases the broad process for transducing and then base editing T cells. For the synthetic RNA molecules, the striking factor for the results were the major advantages that were observed from the two-part (crRNA:tracrRNA) and single part (sgRNA) guide systems, where there was a considerable advantage gained from using a single synthetic guide RNA molecule compared to the two-part guides. In particular, the one-part guide system showed considerable delivery advantages with a dramatic increase in multiplex editing (FIG. 11).

Additionally, with the systematic improvements to the workflow for T cells it is shown that the RNA mediated base editing system can multiplex edit targets at 70-90% efficiency with minimal donor variation (FIG. 12,13), and is therefore of high clinical relevance and the workflow generated can be used directly for therapies. Additionally, the RNA mediated base editing technology shows that the system has an unexpectedly low indel rate compared to the nuclease Cas9, with exceptionally high deamination purity (low C to G/A percentage) (FIG. 14). Furthermore, when assessing off-targets the RNA mediated base editing system shows surprising advantages against double strand DNA break technology (Cas9 nuclease active), where the mismatches of off-target sites are less tolerated for the base editing technology with stark differences observed between the technologies. For example FIG. 16 shows that Cas9 nuclease activity has high amounts of off-target indels (FIG. 16A) and RNA mediated base editing system shows barely detectable indels and low deamination events (FIG. 16B); this result was unexpected as it shows that this technology has much better safety profile for guide-specific off-target events, on the genome, compared to that as nuclease active CRISPR technologies.

The exemplified RNA mediated base editing system has displayed high efficacy but has also been shown that the functionality of the T cells is unaffected by the base editing manipulation, with the edited cells (FIG. 17) showing unaffected tumour cell killing (FIG. 18) and able to correctly secrete cytokines in response to tumour cell killing (FIG. 19). Additionally, given that allogeneic therapies are commonly applied in conjunction of chimeric antigen receptor (CAR) technology, we have exemplified that you can include a virally introduced CAR and then multiplex edit T cells with equivalent to that in non-transduced cells.

The RNA mediated base editing system has been shown with a large body of evidence that it can be used safety and effectively in T cells, and we further display that the technology is not limited to work on primary cells but can also be used on stem cells derived from human. In FIGS. 23 and 24 we show human induced pluripotent stem cells (iPSCs) can be genetically modified using the technology with similar efficiency to that displayed in primary human cells. This showcases the technology can be utilised in stem cells that can be readily differentiated in multiple therapeutically relevant lineages. In conclusion, it has been displayed in the body of this document that the versatile modular RNA mediated base editing technology can be safely applied, for singleplex and multiplex gene editing, in therapeutically relevant systems with high efficacy in human immune and stem cells.

Table 5: Single-guide RNAs (sgRNAs) for TRAC, TRBC1, TRBC2, PDCD1, CD52 and B2M Functional Knock-Out Base Editing

An example list of guides designs for both sgRNA and crRNA formats that can create a functional knock-out using the base editing technology exemplified. The list includes guides specific to the introduction of a premature stop codon and splice disruption sites, which were generated by in-house proprietary software.

| Gene Name | Guide ID | KO Type* | Strand | Guide Sequence | PAM |
|---|---|---|---|---|---|
| B2M | B2M_1 | Stop | sense | CACAGCCCAAGATAGT | TGG |
| | | | | TAAG (SEQ ID NO: 61) | |
| | B2M_2 | Stop | sense | ACAGCCCAAGATAGTT | GGG |
| | | | | AAGT (SEQ ID NO: 62) | |
| | B2M_3 | Stop | anti | TTACCCCACTTAACTA | GGG |
| | | | | TCTT (SEQ ID NO: 63) | |
| | B2M_4 | Stop | anti | CTTACCCCACTTAACT | TGG |
| | | | | ATCT (SEQ ID NO: 64) | |
| | B2M_5 | Splice | anti | ACTCACGCTGGATAGC | AGG |
| | | | | CTCC (SEQ ID NO: 65) | |
| | B2M_6 | Splice | anti | TTGGAGTACCTGAGGA | CGG |
| | | | | ATAT (SEQ ID NO: 66) | |
| | B2M_7 | Splice | anti | TCGATCTATGAAAAAG | TGG |
| | | | | ACAG (SEQ ID NO: 67) | |
| | B2M_8 | Splice | anti | AACCTGAAAAGAAAA | AGG |
| | | | | GAAAA (SEQ ID NO: 68) | |
| CD52 | CD52_1 | Stop | sense | GTACAGGTAAGAGCAA | TGG |
| | | | | CGCC (SEQ ID NO: 69) | |
| | CD52_2 | Stop | sense | CTCCTCCTACAGATAC | TGG |
| | | | | AAAC (SEQ ID NO: 70) | |
| | CD52_3 | Stop | sense | CAGATACAAACTGGAC | AGG |
| | | | | TCTC (SEQ ID NO: 71) | |
| | CD52_4 | Splice | anti | CTCTTACCTGTACCATA | AGG |
| | | | | ACC (SEQ ID NO: 72) | |
| | CD52_5 | Splice | anti | GTATCTGTAGGAGGAG | GGG |
| | | | | AAGT (SEQ ID NO: 73) | |
| | CD52_6 | Splice | anti | TGTATCTGTAGGAGGA | TGG |
| | | | | GAAG (SEQ ID NO: 74) | |
| | CD52_7 | Splice | anti | GTCCAGTTTGTATCTGT | AGG |
| | | | | AGG (SEQ ID NO: 75) | |
| TRAC | TRAC_1 | Stop | sense | AACAAATGTGTCACAA | AGG |
| | | | | AGTA (SEQ ID NO: 76) | |
| | TRAC_2 | Stop | sense | CTTCTTCCCCAGCCCAG | AGG |
| | | | | GTA (SEQ ID NO: 77) | |
| | TRAC_3 | Stop | sense | TTCTTCCCCAGCCCAG | GGG |
| | | | | GTAA (SEQ ID NO: 78) | |
| | TRAC_4 | Stop | sense | AGCCCAGGTAAGGGCA | TGG |
| | | | | GCTT (SEQ ID NO: 79) | |
| | TRAC_5 | Stop | sense | TTTCAAAACCTGTCAG | TGG |
| | | | | TGAT (SEQ ID NO: 80) | |
| | TRAC_6 | Stop | sense | TTCAAAACCTGTCAGT | GGG |
| | | | | GATT (SEQ ID NO: 81) | |
| | TRAC_7 | Stop | sense | CCGAATCCTCCTCCTG | TGG |
| | | | | AAAG (SEQ ID NO: 82) | |
| | TRAC_8 | Splice | Anti | CTTACCTGGGCTGGGG | AGG |
| | | | | AAGA (SEQ ID NO: 83) | |
| | TRAC_9 | Splice | Anti | TTCGTATCTGTAAAAC | AGG |
| | | | | CAAG (SEQ ID NO: 84) | |
| TRBC1/2 | TRBC1/2_1 | Stop | sense | CCACACCCAAAAGGCC | TGG |
| | | | | ACAC (SEQ ID NO: 85) | |
| | TRBC1/2_2 | Stop | Anti | CCCACCAGCTCAGCTC | TGG |
| | | | | CACG (SEQ ID NO: 86) | |
| | TRBC1/2_3 | Stop | sense | CGCTGTCAAGTCCAGT | CGG |
| | | | | TCTA (SEQ ID NO: 87) | |
| | TRBC1/2_4 | Stop | sense | GCTGTCAAGTCCAGTT | GGG |
| | | | | CTAC (SEQ ID NO: 88) | |
| | TRBC1/2_5 | Stop | sense | AGTCCAGTTCTACGGG | CGG |
| | | | | CTCT (SEQ ID NO: 89) | |
| | TRBC1/2_6 | Stop | sense | CACCCAGATCGTCAGC | AGG |
| | | | | GCCG (SEQ ID NO: 90) | |
| | TRBC1/2_7 | Splice | Anti | ACCTGCTCTACCCCAG | CGG |
| | | | | GCCT (SEQ ID NO: 91) | |
| | TRBC1/2_8 | Splice | Anti | CCACTCACCTGCTCTAC | AGG |
| | | | | CCC (SEQ ID NO: 92) | |
| TRBC1 | TRBC1_1 | Stop | sense | CACGGACCCGCAGCCC | AGG |
| | | | | CTCA (SEQ ID NO: 93) | |
| | TRBC1_2 | Stop | Anti | GCGGGGGTTCTGCCAG | TGG |
| | | | | AAGG (SEQ ID NO: 94) | |
| | TRBC1_3 | Stop | Anti | GTTGCGGGGGTTCTGC | AGG |
| | | | | CAGA (SEQ ID NO: 95) | |
| | TRBC1_4 | Stop | sense | ATGACGAGTGGACCCA | AGG |
| | | | | GGAT (SEQ ID NO: 96) | |
| | TRBC1_5 | Stop | sense | TGACGAGTGGACCCAG | GGG |
| | | | | GATA (SEQ ID NO: 97) | |
| | TRBC1_6 | Stop | anti | ACCTGCTCTACCCCAG | CGG |
| | | | | GCCT (SEQ ID NO: 98) | |
| | TRBC1_7 | Stop | sense | CCAACAGTGTCCTACC | AGG |
| | | | | AGCA (SEQ ID NO: 99) | |
| | TRBC1_8 | Stop | sense | CAACAGTGTCCTACCA | GGG |
| | | | | GCAA ( SEQ ID NO: 100) | |
| | TRBC1_9 | Stop | sense | AACAGTGTCCTACCAG | GGG |
| | | | | CAAG (SEQ ID NO: 101) | |
| | TRBC1_10 | Splice | anti | GTCTGAAAGAAAGCAG | AGG |
| | | | | GGAG (SEQ ID NO: 102) | |
| | TRBC1_11 | Splice | anti | CCACAGTCTGAAAGAA | GGG |
| | | | | AGCA (SEQ ID NO: 103) | |
| | TRBC1_12 | Splice | anti | GCCACAGTCTGAAAGA | AGG |
| | | | | AAGC (SEQ ID NO: 104) | |
| | TRBC1_13 | Splice | anti | GACACTGTTGGCACGG | AGG |
| | | | | AGGA (SEQ ID NO: 105) | |
| | TRBC1_14 | Splice | anti | GTAGGACACTGTTGGC | AGG |
| | | | | ACGG (SEQ ID NO: 106) | |
| | TRBC1_15 | Splice | anti | TACCATGGCCATCAAC | GGG |
| | | | | ACAA (SEQ ID NO: 107) | |
| | TRBC1_16 | Splice | anti | TTACCATGGCCATCAA | AGG |
| | | | | CACA (SEQ ID NO: 108) | |
| TRBC2 | TRBC2_1 | Stop | anti | CCAGCTCAGCTCCACG | CGG |
| | | | | TGGT (SEQ ID NO: 109) | |
| | TRBC2_2 | Stop | sense | CACAGACCCGCAGCCC | AGG |
| | | | | CTCA (SEQ ID NO: 110) | |
| | TRBC2_3 | Stop | anti | GCGGGGGTTCTGCCAG | TGG |
| | | | | AAGG (SEQ ID NO: 111) | |
| | TRBC2_4 | Stop | Anti | GTTGCGGGGGTTCTGC | AGG |
| | | | | CAGA (SEQ ID NO: 112) | |
| | TRBC2_5 | Stop | sense | ATGACGAGTGGACCCA | AGG |
| | | | | GGAT (SEQ ID NO: 113) | |
| | TRBC2_6 | Stop | sense | TGACGAGTGGACCCAG | GGG |
| | | | | GATA (SEQ ID NO: 114) | |
| | TRBC2_7 | Stop | Anti | ACCTGCTCTACCCCAG | CGG |
| | | | | GCCT (SEQ ID NO: 115) | |
| | TRBC2_8 | Stop | sense | TCAACAGAGTCTTACC | AGG |
| | | | | AGCA (SEQ ID NO: 116) | |
| | TRBC2_9 | Stop | sense | CAACAGAGTCTTACCA | GGG |
| | | | | GCAA (SEQ ID NO: 117) | |
| | TRBC2_10 | Stop | sense | AACAGAGTCTTACCAG | GGG |
| | | | | CAAG (SEQ ID NO: 118) | |
| | TRBC2_11 | Splice | Anti | CACAGTCTGAAAGAAA | AGG |
| | | | | ACAG (SEQ ID NO: 119) | |
| | TRBC2_12 | Splice | Anti | CCACAGTCTGAAAGAA | AGG |
| | | | | AACA (SEQ ID NO: 120) | |
| | TRBC2_13 | Splice | Anti | GCCACAGTCTGAAAGA | AGG |
| | | | | AAAC (SEQ ID NO: 121) | |
| PDCD1 | PDCD1_1 | Stop | sense | TCCAGGCATGCAGATC | AGG |
| | | | | CCAC (SEQ ID NO: 122) | |
| | PDCD1_2 | Stop | sense | TGCAGATCCCACAGGC | TGG |
| | | | | GCCC (SEQ ID NO: 123) | |
| | PDCD1_3 | Stop | Anti | CGACTGGCCAGGGCGC | GGG |
| | | | | CTGT (SEQ ID NO: 124) | |
| | PDCD1_4 | Stop | Anti | ACGACTGGCCAGGGCG | TGG |
| | | | | CCTG (SEQ ID NO: 125) | |
| | PDCD1_5 | Stop | Anti | ACCGCCCAGACGACTG | GGG |
| | | | | GCCA (SEQ ID NO: 126) | |
| | PDCD1_6 | Stop | Anti | CACCGCCCAGACGACT | AGG |
| | | | | GGCC (SEQ ID NO: 127) | |
| | PDCD1_7 | Stop | Anti | TGTAGCACCGCCCAGA | TGG |
| | | | | CGAC (SEQ ID NO: 128) | |
| | PDCD1_8 | Stop | sense | GGGCGGTGCTACAACT | TGG |
| | | | | GGGC (SEQ ID NO: 129) | |
| | PDCD1_9 | Stop | sense | CGGTGCTACAACTGGG | CGG |
| | | | | CTGG (SEQ ID NO: 130) | |
| | PDCD1_10 | Stop | sense | CTACAACTGGGCTGGC | AGG |
| | | | | GGCC (SEQ ID NO: 131) | |
| | PDCD1_11 | Stop | anti | CACCTACCTAAGAACC | TGG |
| | | | | ATCC (SEQ ID NO: 132) | |
| | PDCD1_12 | Stop | anti | GGGGTTCCAGGGCCTG | GGG |
| | | | | TCTG (SEQ ID NO: 133) | |
| | PDCD1_13 | Stop | anti | GGGGGTTCCAGGGCCT | GGG |
| | | | | GTCT (SEQ ID NO: 134) | |
| | PDCD1_14 | Stop | anti | GGGGGGTTCCAGGGCC | TGG |
| | | | | TGTC (SEQ ID NO: 135) | |
| | PDCD1_15 | Stop | sense | CAGCAACCAGACGGAC | TGG |
| | | | | AAGC (SEQ ID NO: 136) | |
| | PDCD1_16 | Stop | sense | CCCGAGGACCGCAGCC | CGG |
| | | | | AGCC (SEQ ID NO: 137) | |
| | PDCD1_17 | Stop | sense | GGACCGCAGCCAGCCC | AGG |
| | | | | GGCC (SEQ ID NO: 138) | |
| | PDCD1_18 | Stop | sense | CGTGTCACACAACTGC | CGG |
| | | | | CCAA (SEQ ID NO: 139) | |
| | PDCD1_19 | Stop | sense | GTGTCACACAACTGCC | GGG |
| | | | | CAAC (SEQ ID NO: 140) | |
| | PDCD1_20 | Stop | sense | CGCAGATCAAAGAGAG | CGG |
| | | | | CCTG (SEQ ID NO: 141) | |
| | PDCD1_21 | Stop | sense | GCAGATCAAAGAGAGC | GGG |
| | | | | CTGC (SEQ ID NO: 142) | |
| | PDCD1_22 | Stop | sense | AGCCGGCCAGTTCCAA | TGG |
| | | | | ACCC (SEQ ID NO: 143) | |
| | PDCD1_23 | Stop | sense | CGGCCAGTTCCAAACC | TGG |
| | | | | CTGG (SEQ ID NO: 144) | |
| | PDCD1_24 | Stop | sense | CAGTTCCAAACCCTGG | TGG |
| | | | | TGGT (SEQ ID NO: 145) | |
| | PDCD1_25 | Stop | Anti | GGACCCAGACTAGCAG | AGG |
| | | | | CACC (SEQ ID NO: 146) | |
| | PDCD1_26 | Splice | Anti | CACCTACCTAAGAACC | TGG |
| | | | | ATCC (SEQ ID NO: 147) | |
| | PDCD1_27 | Splice | Anti | GGAGTCTGAGAGATGG | AGG |
| | | | | AGAG (SEQ ID NO: 148) | |
| | PDCD1_28 | Splice | Anti | TCTGGAAGGGCACAAA | AGG |
| | | | | GGTC (SEQ ID NO: 149) | |
| | PDCD1_29 | Splice | Anti | TTCTCTCTGGAAGGGC | AGG |
| | | | | ACAA (SEQ ID NO: 150) | |
| | PDCD1_30 | Splice | Anti | TGACGTTACCTCGTGC | CGG |
| | | | | GGCC (SEQ ID NO: 151) | |
| | PDCD1_31 | Splice | Anti | TCCCTGCAGAGAAACA | TGG |
| | | | | CACT (SEQ ID NO: 152) | |
| | PDCD1_32 | Splice | Anti | GAGACTCACCAGGGGC | CGG |
| | | | | TGGC (SEQ ID NO: 153) | |
| | PDCD1_33 | Splice | Anti | TCTTTGAGGAGAAAGG | GGG |
| | | | | GAGA (SEQ ID NO: 154) | |
| | PDCD1_34 | Splice | Anti | TTCTTTGAGGAGAAAG | AGG |
| | | | | GGAG (SEQ ID NO: 155) | |

*Stop = Premature stop codon, Splice = Splice site disruption

1. A method for genetically modifying an immune cell or an iPSC, the method comprising introduction into the cells and/or expression in the cells of:
i) a sequence-targeting component comprising a sequence-targeting protein;
ii) an RNA scaffold comprising
(a) a crRNA comprising a guide RNA sequence that is complementary to a target nucleic acid sequence,
(b) a tracrRNA capable of binding to the sequence-targeting protein, and
(c) an RNA motif; and
(iii) an effector fusion protein comprising
a) an RNA binding domain capable of binding to the RNA motif,
b) a linker, and
c) an effector domain having cytosine deamination activity or adenine deamination activity; and
culturing the introduced cell to produce a genetically modified immune cell or iPSC.

2. A method according to claim 1, wherein the sequence-targeting component comprises the sequence-targeting protein fused to one or more uracil DNA glycosylase (UNG) inhibitor peptide(s) (UGI).

3. (canceled)

4. (canceled)

5. (canceled)

6. The method according to claim 1, wherein the sequence-targeting protein is a Type II Cas protein that is nuclease null or has nickase activity.

7. The method according to claim 1, wherein the sequence-targeting protein comprises the sequence of dCas9 or nCas9.

8. (canceled)

9. The method according to claim 1, wherein the effector domain having cytosine deamination activity is a wild type or genetically engineered version of AID, CDA, APOBEC1, APOBEC3A, APOBEC3B, APOBEC3C, APOBEC3D, APOBEC3F, or other APOBEC family enzymes.

10. The method according to claim 1, wherein the effector domain having adenine deamination activity is a wild type or genetically engineered version of ADA, ADAR family enzymes, or tRNA adenosine deaminases.

11. (canceled)

12. The method of claim 1, wherein the immune cell is selected from a T cell, Natural Killer (NK cell), B cell, or CD34+ hematopoietic stem progenitor cell (HSPC).

13. The method according to claim 1, wherein the immune cell or iPSC comprises a CAR or a TCR.

14. The method of claim 1, wherein the genetic modification corrects a genetic mutation or inactivates the expression of a gene or changes the expression levels of a gene or changes intron-exon splicing.

15. (canceled)

16. (canceled)

17. (canceled)

18. (canceled)

19. The method according to claim 1, wherein the RNA scaffold is chemically modified to comprise 2′-(9-methyl phosphorthioate modification on at least one 5′ nucleotide and at least one 3′ nucleotide of the RNA scaffold sequence.

20. A method according to claim 1, wherein the RNA scaffold is synthesised as two separate components, optionally, wherein the first component comprises a) the crRNA and the second component comprises b) the tracrRNA and c) the RNA motif.

21. (canceled)

22. A method according to claim 1, wherein the RNA scaffold is a synthetic RNA component(s).

23. A method according to claim 1, wherein the RNA motif is located at the 3′ end of the RNA scaffold.

24. (canceled)

25. A method according to claim 1, wherein the RNA motif is an MS2 aptamer, optionally wherein the MS2 aptamer has an extended stem.

26. A method according to claim 1, wherein the sequence targeting fusion protein comprises nCas9 with one or two UGIs and the RNA motif is a single MS2 located at the 3′ end of the RNA scaffold.

27. (canceled)

28. A method according to claim 1, wherein the genetic modification results in reduced expression of any combination of the following proteins TRAC, TRBC1, TRBC2, PDCD1, CD52 and B2M.

29. A method according to claim 1, wherein the method is used for multiplex base editing.

30. (canceled)

31. (canceled)

32. A method according to claim 1 further comprising the step of introducing an exogenous nucleotide sequence into the genome of the genetically modified immune cell or iPSC.

33. (canceled)

34. A genetically modified immune cell or iPSC obtained according to the method of claim 1.

35. A population of genetically modified immune cells or iPSCs obtained according to the method of claim 1, wherein at least 10% of the cells comprise the genetic modification(s).

36. (canceled)


SEQUENCE LISTING (155 sequences)

SEQ ID NO: 1 - protein, 84 aa, Artificial Sequence
  Uracil-DNA glycosylase inhibitor (UGI)
MTNLSDIIEKETGKQLVIQESILMLPEEVEEVIGNKPESDILVHTAYDESTDENVMLLTSDAPEYKPWALVIQDSNGENKIKML

SEQ ID NO: 2 - RNA, 93 nt, Artificial Sequence
  An exemplary hybrid crRNAtracrRNA, gRNA sequence
guuuaagagcuaugcuggaaacagcauagcaaguuuaaauaaggcuaguccguuaucaacuugaaaaaguggcaccgagucggugcuuuuuuu

SEQ ID NO: 3 - RNA, 79 nt, Artificial Sequence
  tracrRNA 1
ggaaccauucaaaacagcauagcaaguuaaaauaaggcuaguccguuaucaacuugaaaaaguggcaccgagucggugc

SEQ ID NO: 4 - RNA, 60 nt, Artificial Sequence
  tracrRNA 2
uagcaaguuaaaauaaggcuaguccguuaucaacuugaaaaaguggcaccgagucggugc

SEQ ID NO: 5 - RNA, 64 nt, Artificial Sequence
  tracrRNA 3
agcauagcaaguuaaaauaaggcuaguccguuaucaacuugaaaaaguggcaccgagucggugc

SEQ ID NO: 6 - RNA, 70 nt, Artificial Sequence
  tracrRNA 4
caaaacagcauagcaaguuaaaauaaggcuaguccguuaucaacuugaaaaaguggcaccgagucggugc

SEQ ID NO: 7 - RNA, 45 nt, Artificial Sequence
  tracrRNA 5
uagcaaguuaaaauaaggcuaguccguuaucaacuugaaaaagug

SEQ ID NO: 8 - RNA, 32 nt, Artificial Sequence
  tracrRNA 6
uagcaaguuaaaauaaggcuaguccguuauca

SEQ ID NO: 9 - RNA, 26 nt, Artificial Sequence
  tracrRNA 7
uagcaaguuaaaauaaggcuaguccg

SEQ ID NO: 10 - RNA, 66 nt, Artificial Sequence
  Telomerase Ku Binding Motif
uucuugucguacuuauagaucgcuacguuauuucaauuuugaaaaucugaguccugggagugcgga

SEQ ID NO: 11 - protein, 1094 aa, Artificial Sequence
  Telomerase Ku Heterodimer
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQDMVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 12 - RNA, 10 nt, Artificial Sequence
  Telomerase Sm7 consensus site (single stranded)
aauuuuugga

SEQ ID NO: 13 - protein, 83 aa, Artificial Sequence
  Monomeric Sm-like protein (archaea)
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 14 - RNA, 19 nt, Artificial Sequence
  MS2 phage operator stem loop
acaugaggaucacccaugu

SEQ ID NO: 15 - protein, 117 aa, Artificial Sequence
  MS2 coat protein
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 16 - RNA, 26 nt, Artificial Sequence
  PP7 phage operator stem loop
auaaggaguuuauauggaaacccuua

SEQ ID NO: 17 - protein, 128 aa, Artificial Sequence
  PP7 coat protein (PCP)
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 18 - RNA, 19 nt, Artificial Sequence
  SfMu Com stem loop
cugaaugccugcgagcauc

SEQ ID NO: 19 - protein, 62 aa, Artificial Sequence
  SfMu Com binding protein
MKSIRCKNCNKLLFKADSFDHIEIRCPRCKRHIIMLNACEHPTEKHCGKREKITHSDETVRY

SEQ ID NO: 20 - protein, 1368 aa, Artificial Sequence
  S. pyogenes dCas9 protein sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1367 aa, Artificial Sequence
  Cas9 D10A Protein
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - DNA, 4101 bp, Artificial Sequence
  DNA encoding Cas9 D10A Protein
gataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 23 - DNA, 110 bp, Artificial Sequence
  RNA scaffold expression cassette (S. pyogenes), containing a 20-nucleotide programmable sequence, a CRISPR RNA motif, and an MS2 operator motif
  misc_feature (1)..(20): 20-nucleotide programmable sequence
ngttttagagctagaaatagcaagttaaaataaggctagtccgttatcaacttgaaaaagtggcaccgagtcggtgcgcgcacatgaggatcacccatgtgctttttttg

SEQ ID NO: 24 - DNA, 157 bp, Artificial Sequence
  RNA scaffold containing two MS2 loops (2xMS2)
gttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttccctttttttctccgct

SEQ ID NO: 25 - protein, 340 aa, Artificial Sequence
  Effector AID -MCP fusion
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 26 - DNA, 594 bp, Artificial Sequence
  wtAID cDNA
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 27 - protein, 198 aa, Artificial Sequence [sequence omitted]

SEQ ID NO: 28 - DNA, 594 bp, Artificial Sequence
  AID_S38A cDNA
atggacagcctcttgatgaaccggaggaagtttctttaccaattcaaaaatgtccgctgggctaagggtcggcgtgagacctacctgtgctacgtagtgaagaggcgtgacgccgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 29 - protein, 198 aa, Artificial Sequence
  AID_S38A protein
MDSLLMNRRKFLYQFKNVRWAKGRRETYLCYVVKRRDAATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 30 - protein, 1835 aa, Artificial Sequence
  Protein sequence of RNA scaffold mediated recruitment system (Generation 1 AID) construct
PKKKRKVMDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 31 - protein, 1941 aa, Artificial Sequence
  Protein sequence of RNA scaffold mediated recruitment system (Generation 2 AID) construct
PKKKRKVMDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 32 - protein, 1866 aa, Artificial Sequence
  Protein sequence of RNA scaffold mediated recruitment system (Generation 1 APOBEC1) construct
PKKKRKVMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 33 - protein, 1866 aa, Artificial Sequence; identical to SEQ ID NO: 32
  Protein sequence of RNA scaffold mediated recruitment system (Generation 2 APOBEC1) construct

SEQ ID NO: 34 - RNA, 127 nt, Artificial Sequence
  RNA sequence of gRNA_MS2 construct
  misc_feature (1)..(19): n′s represent customisable target
nnnnnnnnnnnnnnnnnnnguuuuagagcuagaaauagcaaguuaaaauaaggcuaguccguuaucaacuugaaaaaguggcaccgagucggugcgcgcacaugaggaucacccaugugcuuuuuuu

SEQ ID NO: 35 - DNA, 168 bp, Artificial Sequence
  sequence of gRNA_2xMS2 construct
  misc_feature (1)..(18): n′s represent customisable target
nnnnnnnnnnnnnnnnnnguuuuagagcuagaaauagcaaguuaaaauaaggcuaguccguuaucaacuugaaaaaguggcaccgagucggugcgggagcacaugaggaucacccaugugccacgagcgacaugaggaucacccaugucgctcgtgttcccuuuuuuu

SEQ ID NO: 36 - RNA, 42 nt, Artificial Sequence
  Synthetic crRNA Sequence
  misc_feature (1)..(1): residues 1 and 2 are 2′OMe modified
  misc_feature (1)..(20): n′s denote the 20 base target specifc sequence
  misc_feature (1)..(2): residues 1 and 2 are phosphorothioate modified
nnnnnnnnnnnnnnnnnnnnguuuuagagcuaugcuguuuug

SEQ ID NO: 37 - RNA, 100 nt, Artificial Sequence
  Synthetic 1xMS2 tracrRNA-Aptamer Sequence
  misc_feature (97)..(98): residues 97 and 98 are 2′OMe modified
  misc_feature (97)..(98): residues 97 and 98 are phosphothorothioate modified
aacagcauagcaaguuaaaauaaggcuaguccguuaucaacuugaaaaaguggcaccgagucggugcgcgcacaugaggaucacccaugugcuuuumuuu

SEQ ID NO: 38 - RNA, 142 nt, Artificial Sequence
  Synthetic 2xMS2 tracrRNA-Aptamer Sequence
  misc_feature (139)..(140): residues 139 and 140 are 2′OMe modified
  misc_feature (139)..(140): residues 139 and 140 are phosphothorothioate modified
aacagcauagcaaguuaaaauaaggcuaguccguuaucaacuugaaaaaguggcaccgagucggugcgggagcacaugaggaucacccaugugccacgagcgacaugaggaucacccaugucgcucguguucccuuuumuuu

SEQ ID NO: 39 - RNA, 170 nt, Artificial Sequence
  Lentiviral sgRNA sequences
  misc_feature (1)..(20): n′s denote the 20 base target specifc sequence
nnnnnnnnnnnnnnnnnnnnguuuuagagcuagaaauagcaaguuaaaauaaggcuaguccguuaucaacuugaaaaaguggcaccgagucggugcgggagcacaugaggaucacccaugugccacgagcgacaugaggaucacccaugucgcucguguucccuuuuuuu

SEQ ID NO: 40 - RNA, 42 nt, Artificial Sequence
  TRAC crRNA sequence
  misc_feature (1)..(2): residues 1 and 2 are phosphorothioate modified
  misc_feature (1)..(2): residues 1 and 2 are 2′OMe modified
  misc_feature (1)..(2): residues 1 and 2 are 2′OMe modified
uucguaucuguaaaaccaagguuuuagagcuaugcuguuuug

SEQ ID NO: 41 - RNA, 42 nt, Artificial Sequence
  CD52 crRNA sequence
  misc_feature (1)..(2): residues 1 and 2 are phosphothorothioate modified
  misc_feature (1)..(2): residues 1 and 2 are 2′OMe modified
cucuuaccuguaccauaaccguuuuagagcuaugcuguuuug

SEQ ID NO: 42 - RNA, 42 nt, Artificial Sequence
  B2M crRNA sequence
  misc_feature (1)..(2): residues 1 and 2 are phosphothorothioate modified
  misc_feature (1)..(2): residues 1 and 2 are 2′OMe modified
acucacgcuggauagccuccguuuuagagcuaugcuguuuug

SEQ ID NO: 43 - RNA, 42 nt, Artificial Sequence
  PD-1 crRNA sequence
  misc_feature (1)..(1): residues 1 and 2 are phosphorothioate modified
  misc_feature (1)..(1): residues 1 and 2 are 2′OMe modified
caccuaccuaagaaccauccguuuuagagcuaugcuguuuug

SEQ ID NO: 44 - RNA, 99 nt, Artificial Sequence
  1xMS2 tracrRNA-Aptamer sequence
  misc_feature (97)..(98): residues 97 and 98 are phosphothorothioate modified
  misc_feature (97)..(98): residues 97 and 98 are 2′OMe modified
aacagcauagcaaguuaaaauaaggcuaguccguuaucaacuugaaaaaguggcaccgagucggugcgcgcacaugaggaucacccaugugcuuuuuuu

SEQ ID NO: 45 - RNA, 128 nt, Artificial Sequence
  TRAC sgRNA with 1xMS2 tracrRNA-Aptamer sequence
  misc_feature (1)..(2): residues 1 and 2 are phosphothorothioate modified
  misc_feature (1)..(2): residues 1 and 2 are 2′OMe modified
  misc_feature (127)..(128): residues 127 and 128 are phosphothorothioate modified
  misc_feature (127)..(128): residues 127 and 128 are 2′OMe modified
uucguaucuguaaaaccaagguuuuagagcuagaaauagcaaguuaaaauaaggcuaguccguuaucaacuugaaaaaguggcaccgagucggugcgcgcacaugaggaucacccaugugcuuuuuuu

SEQ ID NO: 46 - RNA, 128 nt, Artificial Sequence
  B2M sgRNA with 1xMS2 tracrRNA-Aptamer sequence
  misc_feature (1)..(2): residues 1 and 2 are phosphothorothioate modified
  misc_feature (1)..(2): residues 1 and 2 are 2′OMe modified
  misc_feature (126)..(127): residues 126 and 127 are phosphothorothioate modified
  misc_feature (126)..(127): residues 126 and 127 are 2′OMe modified
acucacgcuggauagccuccguuuuagagcuagaaauagcaaguuaaaauaaggcuaguccguuaucaacuugaaaaaguggcaccgagucggugcgcgcacaugaggaucacccaugugcuuuuuuu

SEQ ID NO: 47 - RNA, 128 nt, Artificial Sequence
  CD52 sgRNA with 1xMS2 tracrRNA-Aptamer sequence
  misc_feature (1)..(2): residues 1 and 2 are phosphothorothioate modified
  misc_feature (1)..(2): residues 1 and 2 are 2′OMe modified
  misc_feature (126)..(127): residues 126 and 127 are phosphothorothioate modified
  misc_feature (126)..(127): residues 126 and 127 are 2′OMe modified
cucuuaccuguaccauaaccguuuuagagcuagaaauagcaaguuaaaauaaggcuaguccguuaucaacuugaaaaaguggcaccgagucggugcgcgcacaugaggaucacccaugugcuuuuuuu

SEQ ID NO: 48 - RNA, 128 nt, Artificial Sequence
  PD-1 sgRNA with 1xMS2 tracrRNA-Aptamer sequence
  misc_feature (1)..(2): residues 1 and 2 are phosphothorothioate modified
  misc_feature (1)..(2): residues 1 and 2 are 2′OMe modified
  misc_feature (126)..(127): residues 126 and 127 are phosphothorothioate modified
  misc_feature (126)..(127): residues 126 and 127 are 2′OMe modified
caccuaccuaagaaccauccguuuuagagcuagaaauagcaaguuaaaauaaggcuaguccguuaucaacuugaaaaaguggcaccgagucggugcgcgcacaugaggaucacccaugugcuuuuuuu

SEQ ID NO: 49 - DNA, 53 bp, Artificial Sequence
  B2M target locus forward primer
tgactggagttcagacgtgtgctcttccgatctggccttgtcctgattggctg

SEQ ID NO: 50 - DNA, 51 bp, Artificial Sequence
  B2M target locus reverse primer
acactctttccctacacgacgctcttccgatctcgcttccccgagatccag

SEQ ID NO: 51 - DNA, 53 bp, Artificial Sequence
  CD52 target locus forward primer
tgactggagttcagacgtgtgctcttccgatctaagctgctaccaagacagcc

SEQ ID NO: 52 - DNA, 53 bp, Artificial Sequence
  CD52 target locus reverse primer
acactctttccctacacgacgctcttccgatctcaggtttctctcagggcagc

SEQ ID NO: 53 - DNA, 54 bp, Artificial Sequence
  TRAC target locus forward primer
tgactggagttcagacgtgtgctcttccgatctggggatatgcacagaagctgc

SEQ ID NO: 54 - DNA, 54 bp, Artificial Sequence
  TRAC target locus reverse primer
acactctttccctacacgacgctcttccgatctctcagagcttaggatgcaccc

SEQ ID NO: 55 - DNA, 52 bp, Artificial Sequence
  PDCD1 target locus forward primer
tgactggagttcagacgtgtgctcttccgatctggcaccctcccttcaacct

SEQ ID NO: 56 - DNA, 51 bp, Artificial Sequence
  PDCD1 target locus reverse primer
acactctttccctacacgacgctcttccgatctctccagacccctcgctcc

SEQ ID NO: 57 - DNA, 51 bp, Artificial Sequence
  OT1 target locus forward primer
acactctttccctacacgacgctcttccgatcttgccttcccagacccaga

SEQ ID NO: 58 - DNA, 50 bp, Artificial Sequence
  OT1 target locus reverse primer
tgactggagttcagacgtgtgctcttccgatctagatcaggctggcgggc

SEQ ID NO: 59 - DNA, 53 bp, Artificial Sequence
  OT2 target locus forward primer
tgactggagttcagacgtgtgctcttccgatctcatgatagcagccacaagaa

SEQ ID NO: 60 - DNA, 53 bp, Artificial Sequence
  OT2 target locus reverse primer
acactctttccctacacgacgctcttccgatctagcccatgcggagagatcat

SEQ ID NO: 61 - DNA, 20 bp, Artificial Sequence
  B2M_1 sgRNA guide sequence
cacagcccaagatagttaag

SEQ ID NO: 62 - DNA, 20 bp, Artificial Sequence
  B2M_2 sgRNA guide sequence
acagcccaagatagttaagt

SEQ ID NO: 63 - DNA, 20 bp, Artificial Sequence
  B2M_3 sgRNA guide sequence
ttaccccacttaactatctt

SEQ ID NO: 64 - DNA, 20 bp, Artificial Sequence
  B2M_4 sgRNA guide sequence
cttaccccacttaactatct

SEQ ID NO: 65 - DNA, 20 bp, Artificial Sequence
  B2M_5 sgRNA guide sequence
actcacgctggatagcctcc

SEQ ID NO: 66 - DNA, 20 bp, Artificial Sequence
  B2M_6 sgRNA guide sequence
ttggagtacctgaggaatat

SEQ ID NO: 67 - DNA, 20 bp, Artificial Sequence
  B2M_7 sgRNA guide sequence
tcgatctatgaaaaagacag

SEQ ID NO: 68 - DNA, 20 bp, Artificial Sequence
  B2M_8 sgRNA guide sequence
aacctgaaaagaaaagaaaa

SEQ ID NO: 69 - DNA, 20 bp, Artificial Sequence
  CD52_1 sgRNA guide sequence
gtacaggtaagagcaacgcc

SEQ ID NO: 70 - DNA, 20 bp, Artificial Sequence
  CD52_2 sgRNA guide sequence
ctcctcctacagatacaaac

SEQ ID NO: 71 - DNA, 20 bp, Artificial Sequence
  CD52_3 sgRNA guide sequence
cagatacaaactggactctc

SEQ ID NO: 72 - DNA, 20 bp, Artificial Sequence
  CD52_4 sgRNA guide sequence
ctcttacctgtaccataacc

SEQ ID NO: 73 - DNA, 20 bp, Artificial Sequence
  CD52_5 sgRNA guide sequence
gtatctgtaggaggagaagt

SEQ ID NO: 74 - DNA, 20 bp, Artificial Sequence
  CD52_6 sgRNA guide sequence
tgtatctgtaggaggagaag

SEQ ID NO: 75 - DNA, 20 bp, Artificial Sequence
  CD52_7 sgRNA guide sequence
gtccagtttgtatctgtagg

SEQ ID NO: 76 - DNA, 20 bp, Artificial Sequence
  TRAC_1 sgRNA guide sequence
aacaaatgtgtcacaaagta

SEQ ID NO: 77 - DNA, 20 bp, Artificial Sequence
  TRAC_2 sgRNA guide sequence
cttcttccccagcccaggta

SEQ ID NO: 78 - DNA, 20 bp, Artificial Sequence
  TRAC_3 sgRNA guide sequence
ttcttccccagcccaggtaa

SEQ ID NO: 79 - DNA, 20 bp, Artificial Sequence
  TRAC_4 sgRNA guide sequence
agcccaggtaagggcagctt

SEQ ID NO: 80 - DNA, 20 bp, Artificial Sequence
  TRAC_5 sgRNA guide sequence
tttcaaaacctgtcagtgat

SEQ ID NO: 81 - DNA, 20 bp, Artificial Sequence
  TRAC_6 sgRNA guide sequence
ttcaaaacctgtcagtgatt

SEQ ID NO: 82 - DNA, 20 bp, Artificial Sequence
  TRAC_7 sgRNA guide sequence
ccgaatcctcctcctgaaag

SEQ ID NO: 83 - DNA, 20 bp, Artificial Sequence
  TRAC_8 sgRNA guide sequence
cttacctgggctggggaaga

SEQ ID NO: 84 - DNA, 20 bp, Artificial Sequence
  TRAC_9 sgRNA guide sequence
ttcgtatctgtaaaaccaag

SEQ ID NO: 85 - DNA, 20 bp, Artificial Sequence
  TRBC1/2_1 sgRNA guide sequence
ccacacccaaaaggccacac

SEQ ID NO: 86 - DNA, 20 bp, Artificial Sequence
  TRBC1/2_2 sgRNA guide sequence
cccaccagctcagctccacg

SEQ ID NO: 87 - DNA, 20 bp, Artificial Sequence
  TRBC1/2_3 sgRNA guide sequence
cgctgtcaagtccagttcta

SEQ ID NO: 88 - DNA, 20 bp, Artificial Sequence
  TRBC1/2_4 sgRNA guide sequence
gctgtcaagtccagttctac

SEQ ID NO: 89 - DNA, 20 bp, Artificial Sequence
  TRBC1/2_5 sgRNA guide sequence
agtccagttctacgggctct

SEQ ID NO: 90 - DNA, 20 bp, Artificial Sequence
  TRBC1/2_6 sgRNA guide sequence
cacccagatcgtcagcgccg

SEQ ID NO: 91 - DNA, 20 bp, Artificial Sequence
  TRBC1/2_7 sgRNA guide sequence
acctgctctaccccaggcct

SEQ ID NO: 92 - DNA, 20 bp, Artificial Sequence
  TRBC1/2_8 sgRNA guide sequence
ccactcacctgctctacccc

SEQ ID NO: 93 - DNA, 20 bp, Artificial Sequence
  TRBC1_1 sgRNA guide sequence
cacggacccgcagcccctca

SEQ ID NO: 94 - DNA, 20 bp, Artificial Sequence
  TRBC1_2 sgRNA guide sequence
gcgggggttctgccagaagg

SEQ ID NO: 95 - DNA, 20 bp, Artificial Sequence
  TRBC1_3 sgRNA guide sequence
gttgcgggggttctgccaga

SEQ ID NO: 96 - DNA, 20 bp, Artificial Sequence
  TRBC1_4 sgRNA guide sequence
atgacgagtggacccaggat

SEQ ID NO: 97 - DNA, 20 bp, Artificial Sequence
  TRBC1_5 sgRNA guide sequence
tgacgagtggacccaggata

SEQ ID NO: 98 - DNA, 20 bp, Artificial Sequence
  TRBC1_6 sgRNA guide sequence
acctgctctaccccaggcct

SEQ ID NO: 99 - DNA, 20 bp, Artificial Sequence
  TRBC1_7 sgRNA guide sequence
ccaacagtgtcctaccagca

SEQ ID NO: 100 - DNA, 20 bp, Artificial Sequence
  TRBC1_8 sgRNA guide sequence
caacagtgtcctaccagcaa

SEQ ID NO: 101 - DNA, 20 bp, Artificial Sequence
  TRBC1_9 sgRNA guide sequence
aacagtgtcctaccagcaag

SEQ ID NO: 102 - DNA, 20 bp, Artificial Sequence
  TRBC1_10 sgRNA guide sequence
gtctgaaagaaagcagggag

SEQ ID NO: 103 - DNA, 20 bp, Artificial Sequence
  TRBC1_11 sgRNA guide sequence
ccacagtctgaaagaaagca

SEQ ID NO: 104 - DNA, 20 bp, Artificial Sequence
  TRBC1_12 sgRNA guide sequence
gccacagtctgaaagaaagc

SEQ ID NO: 105 - DNA, 20 bp, Artificial Sequence
  TRBC1_13 sgRNA guide sequence
gacactgttggcacggagga

SEQ ID NO: 106 - DNA, 20 bp, Artificial Sequence
  TRBC1_14 sgRNA guide sequence
gtaggacactgttggcacgg

SEQ ID NO: 107 - DNA, 20 bp, Artificial Sequence
  TRBC1_15 sgRNA guide sequence
taccatggccatcaacacaa

SEQ ID NO: 108 - DNA, 20 bp, Artificial Sequence
  TRBC1_16 sgRNA guide sequence
ttaccatggccatcaacaca

SEQ ID NO: 109 - DNA, 20 bp, Artificial Sequence
  TRBC2_1 sgRNA guide sequence
ccagctcagctccacgtggt

SEQ ID NO: 110 - DNA, 20 bp, Artificial Sequence
  TRBC2_2 sgRNA guide sequence
cacagacccgcagcccctca

SEQ ID NO: 111 - DNA, 20 bp, Artificial Sequence
  TRBC2_3 sgRNA guide sequence
gcgggggttctgccagaagg

SEQ ID NO: 112 - DNA, 20 bp, Artificial Sequence
  TRBC2_4 sgRNA guide sequence
gttgcgggggttctgccaga

SEQ ID NO: 113 - DNA, 20 bp, Artificial Sequence
  TRBC2_5 sgRNA guide sequence
atgacgagtggacccaggat

SEQ ID NO: 114 - DNA, 20 bp, Artificial Sequence
  TRBC2_6 sgRNA guide sequence
tgacgagtggacccaggata

SEQ ID NO: 115 - DNA, 20 bp, Artificial Sequence
  TRBC2_7 sgRNA guide sequence
acctgctctaccccaggcct

SEQ ID NO: 116 - DNA, 20 bp, Artificial Sequence
  TRBC2_8 sgRNA guide sequence
tcaacagagtcttaccagca

SEQ ID NO: 117 - DNA, 20 bp, Artificial Sequence
  TRBC2_9 sgRNA guide sequence
caacagagtcttaccagcaa

SEQ ID NO: 118 - DNA, 20 bp, Artificial Sequence
  TRBC2_10 sgRNA guide sequence
aacagagtcttaccagcaag

SEQ ID NO: 119 - DNA, 20 bp, Artificial Sequence
  TRBC2_11 sgRNA guide sequence
cacagtctgaaagaaaacag

SEQ ID NO: 120 - DNA, 20 bp, Artificial Sequence
  TRBC2_12 sgRNA guide sequence
ccacagtctgaaagaaaaca

SEQ ID NO: 121 - DNA, 20 bp, Artificial Sequence
  TRBC2_13 sgRNA guide sequence
gccacagtctgaaagaaaac

SEQ ID NO: 122 - DNA, 20 bp, Artificial Sequence
  PDCD1_1 sgRNA guide sequence
tccaggcatgcagatcccac

SEQ ID NO: 123 - DNA, 20 bp, Artificial Sequence
  PDCD1_2 sgRNA guide sequence
tgcagatcccacaggcgccc

SEQ ID NO: 124 - DNA, 20 bp, Artificial Sequence
  PDCD1_3 sgRNA guide sequence
cgactggccagggcgcctgt

SEQ ID NO: 125 - DNA, 20 bp, Artificial Sequence
  PDCD1_4 sgRNA guide sequence
acgactggccagggcgcctg

SEQ ID NO: 126 - DNA, 20 bp, Artificial Sequence
  PDCD1_5 sgRNA guide sequence
accgcccagacgactggcca

SEQ ID NO: 127 - DNA, 20 bp, Artificial Sequence
  PDCD1_6 sgRNA guide sequence
caccgcccagacgactggcc

SEQ ID NO: 128 - DNA, 20 bp, Artificial Sequence
  PDCD1_7 sgRNA guide sequence
tgtagcaccgcccagacgac

SEQ ID NO: 129 - DNA, 20 bp, Artificial Sequence
  PDCD1_8 sgRNA guide sequence
gggcggtgctacaactgggc

SEQ ID NO: 130 - DNA, 20 bp, Artificial Sequence
  PDCD1_9 sgRNA guide sequence
cggtgctacaactgggctgg

SEQ ID NO: 131 - DNA, 20 bp, Artificial Sequence
  PDCD1_10 sgRNA guide sequence
ctacaactgggctggcggcc

SEQ ID NO: 132 - DNA, 20 bp, Artificial Sequence
  PDCD1_11 sgRNA guide sequence
cacctacctaagaaccatcc

SEQ ID NO: 133 - DNA, 20 bp, Artificial Sequence
  PDCD1_12 sgRNA guide sequence
ggggttccagggcctgtctg

SEQ ID NO: 134 - DNA, 20 bp, Artificial Sequence
  PDCD1_13 sgRNA guide sequence
gggggttccagggcctgtct

SEQ ID NO: 135 - DNA, 20 bp, Artificial Sequence
  PDCD1_14 sgRNA guide sequence
ggggggttccagggcctgtc

SEQ ID NO: 136 - DNA, 20 bp, Artificial Sequence
  PDCD1_15 sgRNA guide sequence
cagcaaccagacggacaagc

SEQ ID NO: 137 - DNA, 20 bp, Artificial Sequence
  PDCD1_16 sgRNA guide sequence
cccgaggaccgcagccagcc

SEQ ID NO: 138 - DNA, 20 bp, Artificial Sequence
  PDCD1_17 sgRNA guide sequence
ggaccgcagccagcccggcc

SEQ ID NO: 139 - DNA, 20 bp, Artificial Sequence
  PDCD1_18 sgRNA guide sequence
cgtgtcacacaactgcccaa

SEQ ID NO: 140 - DNA, 20 bp, Artificial Sequence
  PDCD1_19 sgRNA guide sequence
gtgtcacacaactgcccaac

SEQ ID NO: 141 - DNA, 20 bp, Artificial Sequence
  PDCD1_20 sgRNA guide sequence
cgcagatcaaagagagcctg

SEQ ID NO: 142 - DNA, 20 bp, Artificial Sequence
  PDCD1_21 sgRNA guide sequence
gcagatcaaagagagcctgc

SEQ ID NO: 143 - DNA, 20 bp, Artificial Sequence
  PDCD1_22 sgRNA guide sequence
agccggccagttccaaaccc

SEQ ID NO: 144 - DNA, 20 bp, Artificial Sequence
  PDCD1_23 sgRNA guide sequence
cggccagttccaaaccctgg

SEQ ID NO: 145 - DNA, 20 bp, Artificial Sequence
  PDCD1_24 sgRNA guide sequence
cagttccaaaccctggtggt

SEQ ID NO: 146 - DNA, 20 bp, Artificial Sequence
  PDCD1_25 sgRNA guide sequence
ggacccagactagcagcacc

SEQ ID NO: 147 - DNA, 20 bp, Artificial Sequence
  PDCD1_26 sgRNA guide sequence
cacctacctaagaaccatcc

SEQ ID NO: 148 - DNA, 20 bp, Artificial Sequence
  PDCD1_27 sgRNA guide sequence
ggagtctgagagatggagag

SEQ ID NO: 149 - DNA, 20 bp, Artificial Sequence
  PDCD1_28 sgRNA guide sequence
tctggaagggcacaaaggtc

SEQ ID NO: 150 - DNA, 20 bp, Artificial Sequence
  PDCD1_29 sgRNA guide sequence
ttctctctggaagggcacaa

SEQ ID NO: 151 - DNA, 20 bp, Artificial Sequence
  PDCD1_30 sgRNA guide sequence
tgacgttacctcgtgcggcc

SEQ ID NO: 152 - DNA, 20 bp, Artificial Sequence
  PDCD1_31 sgRNA guide sequence
tccctgcagagaaacacact

SEQ ID NO: 153 - DNA, 20 bp, Artificial Sequence
  PDCD1_32 sgRNA guide sequence
gagactcaccaggggctggc

SEQ ID NO: 154 - DNA, 20 bp, Artificial Sequence
  PDCD1_33 sgRNA guide sequence
tctttgaggagaaagggaga

SEQ ID NO: 155 - DNA, 20 bp, Artificial Sequence
  PDCD1_34 sgRNA guide sequence
ttctttgaggagaaagggag